(19) United States
(12) Patent Application Publication
(10) Pub. No.: US 2025/0138020 A1
(43) Pub. Date: May 1, 2025

(54) Simultaneous Cell Tagging Methods

(71) Applicant: Narwhal Bio, Inc., San Diego, CA (US)

(72) Inventors: Behrad Azimi, San Diego, CA (US); John M. Beierle, San Diego, CA (US)

(73) Assignee: Narwhal Bio, Inc., San Diego, CA (US)

(21) Appl. No.: 18/838,209

(22) PCT Filed: Feb. 13, 2023
(86) PCT No.: PCT/US2023/062516

§ 371 Date: Aug. 13, 2024

Related U.S. Application Data
(60) Provisional application No. 63/310,037, filed on Feb. 14, 2022.

(51) Int. Cl. G01N 33/58; G01N 21/64

(52) U.S. Cl. CPC G01N 33/58; G01N 21/6428; G01N 33/582; G01N 2021/6439; G01N 2458/10

(57) ABSTRACT

Provided herein are methods for, inter alia, separating and/or quantitating subpopulations of cells from a population of cells using simultaneous tagging methods. The methods provided are, inter alia, useful for analyzing and modulating selective cell populations as well as identification of targets for therapeutic purposes.

### CROSS-REFERENCES TO RELATED APPLICATIONS

This application claims priority to U.S. Provisional Application No. 63/310,037, filed Feb. 14, 2022, the disclosure of which is incorporated herein in its entirety and for all purposes.

### BACKGROUND OF THE INVENTION

The field of single-cell multiomics has developed advanced methods for the generation of proteomics and genomics data. In parallel, with the advent of cell therapies and advanced disease-relevant animal models (e.g., patient-derived xenograft) the need for well-defined and pure populations of live cells has rapidly increased in both clinical applications and preclinical research. Meanwhile, large-scale single-cell phenotyping technologies have also advanced to provide functional and disease-relevant single-cell characterization. However, technologies that join single-cell phenotyping with single-cell genomic or proteomics, or enable the purification of live cells based on single-cell phenotypes for in vivo applications at high throughput are lacking. Expanding single-cell multiomics to include single-cell phenotypes as well as genomics or proteomics will make the approach evermore disease-relevant, knowledge-intensive, and a valuable technological tool toward understanding complex diseases. Enabling high throughput live cell purification based on single-cell phenotyping could vastly improve methods of engineering and selecting engineered cells for cell therapies, further advancing animal models that leverage previously unappreciated cell heterogeneities.

Droplet-based methods (DropSeq, Chromium from 10×, others) and methods that enable cell separation into wells of various dimensions help facilitate the collection of proteomics and nucleic acid data en masse, but do not have a concurrent high-throughput method for analyzing cell phenotypes. Similarly, due to throughput limitations or incompatibilities, live cell purification processes do not often employ phenotypic characterization methods that provide in-depth single-cell insight. For example, technologies such as FACS allow for high throughput cell separation but offer very limited phenotyping, where all spatial resolution and cell-cell interaction information is lost. Yet, technologies such as High Content Screening enable in-depth assessment of cell phenotype, including single-cell attributes, cell-cell interactions and spatial resolution, but are generally not compatible with downstream separation techniques that preserve the identity of cells or cell populations. There is a remarkable gap in compatibility across technologies that allow the identification of cell phenotypes and cell-cell interactions at scale and those that provide genomic and proteomic data. Unifying these datasets using live cells in vivo while preserving the identity of individual cells and cell populations throughout the whole process would fulfill a unmet need toward improving human health.

### BRIEF SUMMARY OF THE INVENTION

In an aspect, a method of irradiating a selected sub-population of cells within a population of cells is provided. The method includes: a) simultaneously irradiating each of a first selected sub-population of cells within a population of cells within a first digital image of a first microscope field of view with a first dose of light, thereby forming a first irradiated sub-population of cells and a remainder of cells within the population of cells. At least a portion of the remainder of cells within the population of cells are labeled with the same photosensitive label as the first selected sub-population of cells. A portion of the first selected sub-population of cells includes a first cellular phenotype not present in a portion of the remainder of cells within the population of cell. And b) quantitating the first irradiated sub-population of cells or separating the first irradiated sub-population of cells from the remainder of cells.

In an aspect, a method of selecting a sub-population of cells within a population of cells is provided. The method includes: a) simultaneously irradiating each of a first selected sub-population of cells within a population of cells within a first digital image of a first microscope field of view with a first dose of light, thereby forming a first non-irradiated sub-population of cells and a remainder of cells within the population of cells. At least a portion of the remainder of cells within the population of cells are labeled with the same photosensitive label as the first selected sub-population of cells. A portion of the first selected sub-population of cells includes a first cellular phenotype not present in a portion of the remainder of cells within the population of cells. And b) quantitating the non-irradiated sub-population of cells or separating the non-irradiated sub-population of cells from the remainder of cells.

### BRIEF DESCRIPTION OF THE DRAWINGS

FIG. 1 Depicts steps of the process flow of exemplary embodiments of the methods provided herein.

FIG. 2 Exemplary depiction of the steps involved in the digital image generation useful for the methods provided herein. Step 1: Stitching. Microscopic fields of view are stitched for each acquired channel. Step 2: Cell Segmentation. Utilizing one or more channels and/or planes, including optionally a nuclear channel, cell or nuclear segmentation is performed to create a labeled cell mask that outlines the areas belonging to each cell. Step 3: High Content Analysis. A series of desired attributes (measurements) are made and collected in a Cytometry Table by applying the labeled cell or nuclear mask on each channel, for each cell or a portion thereof. Step 4: Cell Selection. Each cell, based on its attributes and the selection criteria set by user or software, is assigned to a cell population. Step 5: Raw Projection Image. Based on the intended irradiation dose for each population, set by user or software, a Raw Projection Image is created such that the pixels corresponding to a cell have values proportional to the intended irradiation dose for the population to which that cell belongs.

FIG. 3 Exemplary depiction of the steps involved in the tagging procedure using pixel-based processing useful for the methods provided herein. Step A: Imaging. One or more grayscale images in fluorescence or bright field are captured. Step B: Labeling. Cells are stained with a photosensitive label. This step can precede or follow Imaging (Step A). Step C: Tagging. Sample is irradiated with the Digital Image pattern. The arrowhead labeled with the Arabic numeral 1 indicates the Tagging step of pixel-based processing (e.g., thresholding pixel values), while the arrowhead labeled with the Arabic numeral 2 represents the Tagging step of pixel mapping (e.g., cropping, aligning imaging and tagging frames, and/or to correct for optical aberrations). In embodiments, step 1 and step 2 are commutative.

FIG. 4 Exemplary depiction of the steps in the tagging procedure using cell-based processing useful for the methods provided herein. Step A: Imaging. One or more grayscale images in fluorescence or bright field are captured. Step B: Labeling. Cells are stained with a photosensitive label. This step can precede or follow Imaging (Step A). Step C: Tagging. Sample is irradiated with the Digital Image pattern. The arrowhead labeled with the Arabic numeral 1 indicates the Tagging step of cell-based processing (e.g., identify and quantify cells), while the arrowhead labeled with the Arabic numeral 2 represents the Tagging step of pixel mapping (e.g., cropping, aligning imaging and tagging frames, and/or to correct for optical aberrations). In embodiments, step 1 and step 2 are commutative.

FIG. 5A-5B Exemplary depiction of the Projection Image Transformation step useful in methods provided herein. FIG. 5A In embodiments, the transformation includes size and position matching and alignment, including: cropping, (2.a), Euclidean transformations, translation (2.b), reflection (2.c), or rotation (2.d). In embodiments, the transformation includes magnification (uniform and/or non-uniform) and aberration correction using affine transformations: scaling and shear (2.e). FIG. 5B Example transform calibration procedure depicting on the left a calibration pattern image, its uncalibrated transformed image and the resultant projection onto a dense monolayer of cells stained with a fluorescent dye (e.g. DAPI) (top), and the calibrated transform of the calibration pattern, and its projection on the same sample (bottom). Note the lines in the fluorescence images were added in software to aid with alignment. An example Raw Projection Image and the Transformed Projection Image using the calibrated transformation is shown on the right.

FIG. 6 Exemplary examples of mathematical methods used in the pixel interpolation step of the methods provided herein.

FIG. 7A-7B Exemplary embodiments of the Continuous Tagging method provided herein. FIG. 7A: Exemplary depiction of the open-loop Continuous Tagging method provided herein. FIG. 7B: Exemplary depiction of the closed-loop Continuous Tagging method provided herein. See, also Example 5.

FIG. 8A-8F Depiction of embodiments of Simultaneous Tagging method wherein irradiation exposure intensity is maintained constant across different subpopulations. FIG. 8A: Embodiment of Simultaneous Tagging method wherein a projection image is processed into a series of binary projection images, divided in time by the smallest time that the DMD (Digital Micromirror Device) can complete a projection event (dt). FIG. 8B: Embodiment of Simultaneous Tagging method wherein the irradiation exposure start and stop for each subpopulation is staggered and exposures occur sequentially. FIG. 8C: Embodiment of Simultaneous Tagging method wherein the irradiation exposure starts (t_0) simultaneously for all subpopulations but the stop time of the exposure (t_f) is different for each subpopulation. FIG. 8D: Embodiment of Simultaneous Tagging method wherein the irradiation exposure start time is different for each subpopulation, but the end of the irradiation exposure is the same for all subpopulations. FIG. 8E: Embodiment of Simultaneous Tagging method wherein the start and stop times of the irradiation exposure differ across each subpopulation, but the irradiation intensity remains constant for each. The term “ROI” as provided herein refers to a region of interest. In embodiments, the region of interest is an area including the selected sub-population of cells. In embodiments, the region of interest is an area of the selected sub-population of cells. FIG. 8F: Example nuclear fluorescence image (Nuclear Channel) showing all the cells within the cropped area of a field of view and the corresponding phenotype fluorescence image (Phenotype Channel) showing cells of three distinct phenotypes. The Projection Image that was computed to have medium intended irradiation dose for the cells with medium phenotype dye level, and high intended irradiation dose for the cells with high phenotype dye level. Note cells with the low phenotypic dye assigned lowest (e.g. 0) intended irradiation dose are not visible in this image but are nevertheless present as seen in the nuclear image. Three Binary Projection Images calculated using Constant Intensity Simultaneous Tagging as described herein are shown.

FIG. 9A-9C Embodiment of the Simultaneous Tagging method wherein irradiation dose varies through Duty Cycle modulation. FIG. 9A: Embodiment of the Simultaneous Tagging method wherein irradiation exposure start (t_0) and stop (t_f) times are the same but total ON times vary across different subpopulations or ROIs. FIG. 9B: Embodiment of Simultaneous Tagging method wherein the ON times are calculated based on a shared clock or timing signal with a given frequency. FIG. 9C: Embodiment of Simultaneous Tagging wherein the total ON time for each of different subpopulations or ROIs are intended to be different or the same.

FIG. 10 Depiction of an exemplary optical light path and control mechanism useful for methods provided herein. OBJ: Microscopy objective; TL: Tube lens; DAQ: Data acquisition card capable of receiving and generating timing signals; DIC: Multi-pass dichroic; DMD: Digital micromirror device; EM: Emission filter (filter wheel).

FIG. 11 Depiction of exemplary chemical labeling techniques for probing living cells. All tags can be covalently linked or non-covalently linked (e.g. antibodies or membrane binding molecules) to cell surfaces, cell membranes, cell surface proteins, cell surface glycans, as well as internally absorbed specifically or nonspecifically and covalently or non-covalently linked to biomolecules. In addition, expression of a fluorescent protein can act as a labelling technique. Cylinder and trapezoidal shapes represent expressed biomolecules such as DNA, RNA, proteins, and others.

FIG. 12 Depiction of labeling a cell by photo-bleaching a fluorophore useful for the methods provided herein. Step 1: Cells are all labelled with the same fluorescent label. Step 2: Subpopulations of cell phenotypes are identified and irradiated with light at fixed dosing. Step 3: Subpopulations are sorted based on intensity.

FIG. 13 Depiction of an exemplary method for labeling a cell using fluorophore uncaging. Step 1: cells are all labelled with a photo-caged fluorophore. Step 2: Subpopulations are defined by phenotype and selectively irradiated to uncage the fluorophore. Cells can also be photo-bleached to selectively label further phenotypes at this stage akin to methods in the preceding figures. Step 3: Cells are sorted based on fluorescence intensity.

FIG. 14 Depiction of exemplary method for labeling a cell using a photo-releasable fluorophore. Step 1: Fluorophores with photo-cleavable linkers are attached to cells. Step 2: Subpopulations of cells are defined and selectively irradiated for fixed dosages. Step 3: Cells are sorted based on relative fluorescence.

FIG. 15. Depiction of exemplary method for labeling a cell using a photo-activated chemical reaction. Step 1: Fluorophores with photo-reactive linkers are added to cells. The cells may or may not be pre-reacted with a photoactive linker. Step 2: Subpopulations of cells are defined and selectively irradiated for fixed amounts of time. Step 3: Cells are sorted based on relative fluorescence.

FIG. 16 Depiction of exemplary method for labeling a cell using oligonucleotides with blocked 3′ extension. Step 1: Attach 3′ blocked anchor label oligonucleotides to cell surfaces. Step 2: Irradiate with light to remove blocker from selected subpopulation of cells. Add template (single template or a library), polymerase, oligonucleotides and divalent cation and extend to add barcode. Step 3: Wash to remove template and polymerase. Repeat to barcode other cells. In embodiments, the template is not removed.

FIG. 17 Depiction of exemplary method for labeling a cell using photo-caged nucleobases with polymerase extension. Step 1: Attach anchor label oligonucleotides with blocked nucleobases. Step 2: Irradiate with light to remove blockers from the selected subpopulation of cells. Add template (single template or a library), polymerase, oligonucleotides and divalent cation and extend to add barcode. Step 3: Wash to remove template and polymerase. Repeat to barcode other cells. In embodiments, the template is not removed.

FIG. 18 Depiction of exemplary method for labeling a cell using oligonucleotides with a blocked 3′ end followed by Splint Ligation. Step 1: Attach 3′ blocked oligonucleotides to cell surfaces. Some of these oligonucleotides may have a 3′ phosphate attached. Step 2: Irradiate with light to remove blocker from selected subpopulation of cells. Add template and oligo barcodes, ligase and cofactors. Some of the oligonucleotide barcodes may have a 5′ phosphate attached. Step 3: Wash to remove template and enzyme. Repeat to barcode other cells or to barcode these cells a second time. In embodiments, the template is not removed.

FIG. 19 Depiction of exemplary method for labeling a cell using oligonucleotides with blocked nucleobases followed by Splint Ligation. Step 1: Attach nucleobase blocked oligonucleotides to cell surfaces. Some of these oligonucleotides may have a 3′ phosphate attached. Step 2: Irradiate with light to remove blockers from the selected subpopulation of cells. Add template and oligonucleotide barcodes (which may have a 5′ phosphate attached), ligase and cofactors. Step 3: Wash to remove template and enzyme. Repeat to barcode label other cells or these cells a second time. In embodiments, the template is not removed.

FIG. 20 Depiction of exemplary method for labeling a cell using photo-released oligonucleotides. Step 1: Cells are labeled with an oligonucleotide and a photosensitive linker. Step 2: Selected subpopulation of cells are irradiated with light to remove the oligonucleotide.

FIG. 21 Exemplary oligonucleotides useful for labeling a cell using methods provided herein. In embodiments, oligonucleotides do not include UMI (Unique Molecular Identifier) regions. PCR Primer region can also be an adaptor region or a sequencing primer.

FIG. 22 Depiction of exemplary method for labeling a cell by barcoding with polymerase. In embodiments, labels and templates do not include UMI regions. In embodiments, barcodes include location tags, cycle tags, or other relevant temporal, molecular or spatial information. PCR Primer region can also be an adaptor region or a sequencing primer. In embodiments, dehybridize step is omitted.

FIG. 23 Depiction of exemplary method for labeling a cell by barcoding with splint ligation. In embodiments, labels and templates do not include UMI regions. In embodiments, barcodes include location tags, cycle tags, or other relevant temporal, molecular, or spatial information. In embodiments, incoming barcodes have additional photo-labile groups. PCR Primer region can also be an adaptor region or a sequencing primer. In embodiments, dehybridize step is omitted.

FIG. 24 Exemplary barcode outputs after cleavage from the cell after multiple encoding events useful for the methods provided herein. PCR Primer region can also be an adaptor region or a sequencing primer. Amplification of strand's complement directly from the cell surface is also a potential output.

FIG. 25 Exemplary capping steps required for single cell workflows (e.g. 10× genomics) and useful for the methods provided herein. In embodiments, the barcode is introduced already tagged with a 3′-polyA tag.

FIG. 26. Exemplary cell label and capping steps required for downstream sequencing workflows and useful for the methods provided herein. In embodiments, PCR primer sequences are adapter sequences for additional oligo labeling. In embodiments, oligos may not have oligonucleotides.

FIG. 27. Example of simultaneous tagging of multiple phenotypically selected subpopulation of cells An example field of view of a cells imaged using a fluorescence phenotypic imaged (Phenotype Channel). Inset panels illustrate a small area within the same field, and the Nuclear Channel image from a common nuclear dye (e.g. DRAQ5) showing all the cells within that area. The Projection Image that was computed to have medium intended irradiation dose for the cells with medium phenotype dye level, and high intended irradiation dose for the cells with high phenotype dye level. Note cells with the low phenotypic dye assigned lowest (e.g. 0) intended irradiation dose are not visible in this image but are nevertheless present as seen in the nuclear image. The Photocaged Tag Pre-Irradiation showing each cell's the Tag fluorescence before the Irradiation Event and the Photocaged Tag Post-Irradiation image showing each cell's the Tag fluorescence after the Irradiation Event.

FIG. 28A-28D Downstream Processing with Image Cytometry and Flow cytometry. Results of example downstream processing of simultaneously tagged 2 and 3 selected subpopulations of cells using (FIG. 28A and FIG. 28C) flow cytometry and (FIG. 28B and FIG. 28D) image cytometry to assess GREEN (phenotype) and DEEP RED (irradiated tag) fluorescence.

FIG. 29 Photocaged nucleobases with endonuclease cleavage. Step 1—Attach anchor label oligonucleotides with blocked nucleobases. Step 2—Irradiate with light to remove blockers. Add complement (single template or a library), endonuclease, and buffer. Step 3—Wash to remove cleaved oligos. Barcodes can optionally be added on the anchor or after cleavage using described methods.

FIG. 30 Photocaged nucleobases on a template splint. 1—Anchor oligo noncovalently (or covalently) attached to a cell surface has been prehybridized with a photoprotected splint. 2—Irradiating a selected subpopulation of cells and adding an oligo barcode with an optional fluorophore allows for cell selective labelling. 3-n—This process can be repeated with multiple labels, with or without ligation steps to link the barcodes to the anchor stems.

FIG. 31 Example endonuclease cleavage then barcoding. Labels and templates may or may not have UMIs. Barcodes can consist of location tags, cycle tags, or other relevant temporal, molecular or spatial information. In embodiments, barcoding steps are omitted.

### DETAILED DESCRIPTION OF THE INVENTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Definitions

The abbreviations used herein have their conventional meaning within the chemical and biological arts. The chemical structures and formulae set forth herein are constructed according to the standard rules of chemical valency known in the chemical arts.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or branched chain, or combinations thereof, including at least one carbon atom and at least one heteroatom (e.g. O, N, P, Si or S) and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃,
“\* MERGEFORMAT \*
   MERGEFORMAT —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′—
“\* MERGEFORMAT \* MERGEFORMAT represents
   both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC=(O)NHNH₂, —NHC=(O) NH₂, —NHSO₂H, —NHC=(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

As used herein, the term “conjugate” refers to the association between atoms or molecules. The association can be direct or indirect. For example, a conjugate between a nucleic acid and a protein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

Useful reactive moieties or functional groups used for conjugate chemistries (including “click chemistries” as known in the art) herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold;
(h) amine or sulfhydryl groups, which can be, for example, acylated, alkylated or oxidized;
(i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds; and
(n) sulfones, for example, vinyl sulfone.

Chemical synthesis of compositions by joining small modular units using conjugate (“click”) chemistry is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’ Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Trisubstituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stackmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry); “Selective Functionalization of a Genetically Encoded Alkene-Containing Protein via ‘Photoclick Chemistry’ in Bacterial Cells”. Journal of the American Chemical Society 130:9654-9655; Song, Wenjiao and Wang, Yizhong and Qu, Jun and Lin, Qing (2008). “Cu-free click cycloaddition reactions in chemical biology”. Chem Soc Rev 39(4): 1272-1279, Jewett, John C. and Bertozzi, Carolyn R. (2010). “Light-Triggered Click Chemistry”. Chemical Reviews 2021 121 (12), 6991-7031, Srikanth Kumar, Gangnam and Lin, Qing all of which are hereby incorporated by reference in their entirety and for all purposes.

The reactive functional groups can be chosen such that they do not participate in, or interfere with, the chemical stability of the proteins or nucleic acids described herein. By way of example, the nucleic acids can include a vinyl sulfone or other reactive moiety (e.g., maleimide). Optionally, the nucleic acids can include a reactive moiety having the formula —S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

As used herein, the terms “about” and “approximately” can be used interchangeably throughout and refer to a range of values including the specified value, which a person of ordinary skill in the art would consider reasonably similar to the specified value. In embodiments, the terms “about” and “approximately” mean within a standard deviation using measurements generally acceptable in the art. In embodiments, about means a range extending to +/−10% of the specified value. In embodiments, approximately means a range extending to +/−10% of the specified value. In embodiments, about means the specified value. In embodiments, about approximately means the specified value.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

A “chemical linker,” as provided herein, is a covalent linker, a non-covalent linker, a peptide or peptidyl linker (a linker including a peptide moiety), a nucleic acid linker, a polymer, a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof.

The chemical linker as provided herein may be a bond, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein may be a bond, —O—, —S—, —C(O)—, —C(O)O—, —C(O)NH—, —S(O)₂NH—, —NH—, —NHC(O)NH—, —C—O—O— substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered,) heteroarylene, or polymeric make-ups of the groups listed above such as polyamides, polyethlyneglycols, or linked alkyl chains.

In embodiments, the chemical linker is a covalent linker. In embodiments, the chemical linker is a hydrocarbon linker. In embodiments, the chemical linker is a cleavable peptide linker.

Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

A “label” or a “detectable moiety” is a composition detectable by spectroscopic, photochemical, biochemical, immunochemical, chemical, optical, or other physical means. For example, useful labels include ³²P, fluorescent dyes, nucleic acid barcodes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego. Labels or detectable moieties may be covalently or noncovalently attached.

A “photosensitive label” is defined as any molecule, macromolecule, protein, or substituent that undergoes a reaction initiated with light. Photosensitive labels include fluorophores, photocleavable groups, photoblocking groups, photocaged groups, molecular switches (“photoswitches”), photoactivated fluorophores or dyes, photoactivated proteins, fluorescent proteins. Example of fluorescent labels include: Examples of fluorescent labels include, but are not limited to, fluorescein and derivatives thereof, AlexaFluor 647, Alexafluor 488, Atto dyes, 4-acetamido-4-isothiocyanatostilbene-2,2 disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-l-napthyl)maleimide); anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′, 6-diaminidino-2-phenylindole (DAPI); 5′5″-dibromopyrogallol-sulfonaphthalein (Bromopyrogaliol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives; eosin, eosin isothiocyanate, erythrosin and derivatives; erythrosin B, eiythrosin, isothiocyanate; ethidium; fluorescein and derivatives; 5-carboxyfluorescein (FAM), 5-(4,6-dichlorotriazin-2-yl)aminofluorescein (DTAF), 2′, 7-dimethoxy-4′5′-dichloro-6-carboxyfluorescein, fluorescein, fluorescein isothiocyanate, QFITC, (XRITC); fluorescamine; IR144; 446; Malachite Green isothiocyanate; 4-methylumbelliferoneortho cresoiphthalein; nitrotyrosine; pararosaniline; Phenol Red; B-phycoerythiin, o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocvanate, sulforhodamine B, sulforhodamine 101, sulfonvl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives, Cy3; Cy5; Cy5.5; Cy7; RD 700; IRD 800, La Jolta Blue; phthalo cyanine; and naphthaio cyanine. Examples of photoactivatable proteins includes PA-GFP, PA-sfGFP, PAmCherry1, PATagRFP, PAmKate, Phamret, Kaede, Dendra2, mClavGR2, mMaple, PS-CFP2, Meos3.2, EosFP, mEosFP, mEos2, mEos3.2, mEos4a, mEos4b, tdEos, kikGR, PsmORgane, PsmOrange2, mTFP0.7, PDM1-4, Dronpa, Dronpa-2, Dronpa-3, bsDronpa, Padron, Padron0.9, Mut2Q, rsFastLime, rsKame, Dreiklang, mGeos-M, EYQ1, KFP1, rsCherry, rsCherryRev, rsTagRFP, mApple, asFP595, Kindling FP, rseGFP, and rseGFP2. Examples of photoactivatable dyes or fluorophores include PA-JF549, PA-JF-646, DCDHF-based dyes, BODIPY-based dyes, DiR-based photoconvertible dyes, Atto 488, Cy3B, Alexa 647, Cy7, Alexa 750, So-Rhodamine, and additional photoactivated dyes or switches in “Reversible photocontrol of biological systems by the incorporation of molecular photoswitches”. Szymanski, Wiktor and Beierle, John M. and Kistemaker, Hans A. V. and Velema, Willem A. and Feringa, Ben L. 113(8): 6114-61178 (2013) or referenced therein. Photocleavable and photoblocking groups include NVoc and related photolabile derivates as well as para-nitrobenzyl derived molecules.

A “labeled biomolecule” is one that is bound, either covalently, through a linker or a chemical bond, or noncovalently, through ionic, van der Waals, electrostatic, or hydrogen bonds to a label such that the presence of the labeled biomolecule (e.g. protein, polypeptide, nucleic acid, glycan, cell membrane) may be detected by detecting the presence of the label bound to the labeled biomolecule. Alternatively, methods using high affinity interactions may achieve the same results where one of a pair of binding partners binds to the other, e.g., biotin, streptavidin. In embodiments the labeling my take place through post-translational modifications on the protein such as carbohydrates, sugars, or glycans or through epigenetic/epigenetic events on nucleic acids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “CRISPR” or “Clustered Regularly Interspaced Short Palindromic Repeats” is a general term that applies to three types of systems, and system sub-types. In general, the term CRISPR refers to the repetitive regions that encode CRISPR system components (e.g., encoded crRNAs). Three exemplary types of CRISPR systems are depicted in the below Table, each with differing features.

### CRISPR System Types Overview

| System | Features | Examples |
|---|---|---|
| Type I | Multiple proteins (5-7 proteins typical), crRNA, | Staphylococcus epidermidis |
| | requires PAM. DNA Cleavage is catalyzed by Cas3. | (Type IA) |
| Type II | 3-4 proteins (one protein (Cas9) has nuclease | Streptococcus pyogenes |
| | activity) two RNAs, requires PAMs. Target DNA | CRISPR/ Cas9, Francisella |
| | cleavage catalyzed by Cas9 and RNA components. | novicida U112 Cpf1 |
| Type III | Five or six proteins required for cutting, number of | S. epidermidis (Type IIIA); |
| | required RNAs unknown but expected to be 1, PAMs | P. furiosus (Type IIIB). |
| | not required. Type IIIB systems have the ability to | |
| | target RNA. | |

As used herein, “CRISPR complex” refers to the CRISPR proteins and nucleic acid (e.g., RNA) that associate with each other to form an aggregate that has functional activity. An example of a CRISPR complex is a wild type Cas9 (sometimes referred to as Csn1) protein that is bound to a guide RNA specific for a target locus.

As used herein, “CRISPR protein” refers to a protein comprising a nucleic acid (e.g., RNA) binding domain nucleic acid and an effector domain (e.g., Cas9, such as Streptococcus pyogenes Cas9, or CPF1 (cleavage and polyadenylation factor 1)). The nucleic acid binding domains interact with a first nucleic acid molecules either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

CRISPR protein also refers to proteins that form a complex that binds the first nucleic acid molecule referred to above. Thus, one CRISPR protein may bind to, for example, a guide RNA and another protein may have endonuclease activity. These are all considered to be CRISPR proteins because they function as part of a complex that performs the same functions as a single protein, such as Cas9 or CPF1.

A “guide RNA” or “gRNA” as provided herein refers to a ribonucleotide sequence capable of binding a nucleoprotein, thereby forming ribonucleoprotein complex. Likewise a “guide DNA” or “gDNA” as provided herein refers to a deoxyribonucleotide sequence capable of binding a nucleoprotein, thereby forming deoxyribonucleoprotein complex. In embodiments, the guide RNA includes one or more RNA molecules. In embodiments, the guide DNA includes one or more DNA molecules. In embodiments, the gRNA includes a nucleotide sequence complementary to a target site (e.g., a modulator binding sequence). In embodiments, the gDNA includes a nucleotide sequence complementary to a target site (e.g., a modulator binding sequence). The complementary nucleotide sequence may mediate binding of the ribonucleoprotein complex or the deoxyribonucleoprotein complex to said target site thereby providing the sequence specificity of the ribonucleoprotein complex or the deoxyribonucleoprotein complex. Thus, in embodiments, the guide RNA or the guide DNA is complementary to a target nucleic acid (e.g., a modulator binding sequence). In embodiments, the guide RNA binds a target nucleic acid sequence (e.g., a modulator binding sequence). In embodiments, the guide DNA binds a target nucleic acid sequence (e.g., a modulator binding sequence). In embodiments, the guide RNA is complementary to a CRISPR nucleic acid sequence. In embodiments, the complement of the guide RNA or guide DNA has a sequence identity of about 50%, 55%, 60%, 65%, 70%, 75%, 80%, 85%, 90%, 95%, 96%, 97%, 98% or 99% to a target nucleic acid (e.g., a modulator binding sequence). A target nucleic acid sequence as provided herein is a nucleic acid sequence expressed by a cell. In embodiments, the target nucleic acid sequence is an exogenous nucleic acid sequence. In embodiments, the target nucleic acid sequence is an endogenous nucleic acid sequence. In embodiments, the target nucleic acid sequence (e.g., a modulator binding sequence) forms part of a cellular gene. Thus, in embodiments, the guide RNA or guide DNA is complementary to a cellular gene or fragment thereof. In embodiments, the guide RNA or guide DNA is about 50%, 55%, 60%, 65%, 70%, 75%, 80%, 85%, 90%, 95%, 96%, 97%, 98% or 99% to the target nucleic acid sequence (e.g., a modulator binding sequence). In embodiments, the guide RNA or guide DNA is about 50%, 55%, 60%, 65%, 70%, 75%, 80%, 85%, 90%, 95%, 96%, 97%, 98% or 99% complementary to the sequence of a cellular gene. In embodiments, the guide RNA or the guide DNA binds a cellular gene sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a biomolecule such as a receptor, protein, or antibody, antibody variant, antibody region or fragment thereof.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

A “barcode” is any segment of nucleic acid with a unique sequence for a particular phenotype, cell, position, cycle. In some cases barcodes may be present on incoming samples. In some cases barcodes can be linked to one another directly or through nucleic acid spacer regions.

A “UMI” is a unique molecular identifier, similar to a barcode, that allows the unique labelling of steps in a process or molecules to enable downstream identification and/or counting through nucleic acid identification techniques (e.g. hybridization, PCR, sequencing, and qPCR).

A “polymerase” is any enzyme that synthesizes long chains of polymers or nucleic acids. A polymerase as defined here includes any relevant cofactors required for said polymerization include but not limited to divalent cations, nucleoside triphosphates, and or template nucleic acid strands. Typical polymerases include but are not limited to those commercially available from New England Biolabs, Illumina, Lucigen, Sigma Aldrich, Roche as well as their accompanying buffers, reagents, and suggested reactants.

A “ligase” is any enzyme that catalyzes the formation of a covalent bond between two nucleic acids. A ligase as defined here includes any relevant cofactors or reactants required for said reaction including but not limited to cations, oligonucleotide stands, phosphorylated oligonucleotide strands, a template or “splint” to facilitate ligation. Typical ligases include but are not limited to those commercially available from New England Biolabs, Illumina, Lucigen, Sigma Aldrich, Roche as well as their accompanying buffers, reagents, and suggested reactants.

The term “endonuclease” as provided herein refers to enzymes that cleave nucleotides. Non-limiting examples of endonucleases include exonucleases, nucleases, restriction endonucleases, or a combination of thereof. Endonucleases may be sequence specific (i.e., cleaving at a specific site within a nucleotide sequence. Endonucleases may be nonspecific (i.e., cleaving a nucleotide sequence independently of the sequence). Endonucleases may cleave single stranded or double stranded DNA.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. The term “cell” as provided herein includes cells transduced or infected with an agent (e.g., phage, virus) or transfected with a transfection agent (e.g., plasmid, DNA, RNA, siRNA, oligonucleotides). In the context of the present invention, manipulation (e.g., tagging, labeling, selecting) of a cell may include manipulating infectious agents (e.g., viral particles or fragments thereof) that form part of a cell (are inside or attached to a cell) or are included in a cell culture. Thus, the methods provided herein include selection of infectious agents forming part of a cell or a cellular supernatant. Eukaryotic cells include, but are not limited to, yeast cells and cells derived from plants and animals, for example mammalian, insect (e.g., Spodoptera) and human cells. In embodiments, the cell may be a virally infected cell.

A “stem cell” as provided herein refers to a cell characterized by the ability of self-renewal through mitotic cell division and the potential to differentiate into a cell, tissue or an organ with a specific phenotype. Among mammalian stem cells, embryonic stem cells (ES cells) and somatic stem cells (e.g., HSC) can be distinguished. Embryonic stem cells reside in the blastocyst and give rise to embryonic tissues, whereas somatic stem cells reside in adult tissues for the purpose of tissue regeneration and repair.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

“NK cells” or “Natural Killer cells” or “NK lymphocytes” as used herein are a type of lymphocyte (a subtype of white blood cell) that plays a central role in innate and cell-mediated immunity. They can be distinguished from other lymphocytes, such as B cells and T cells, by the presence of NK-cell markers or receptors on the cell surface.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, stem cells, B cells, T cells, NK cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, the sample is obtained for characterization only. In embodiments the sample or a portion thereof is introduced back into a subject of patient after processing.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific cell phenotypes, specific bodily fluids, specific tissues, synoviocytes, synovial fluid, synovial tissue, fibroblast-like synoviocytes, macrophagelike synoviocytes, etc).

The term “control” as used herein further refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition. Non limiting examples include humans, other mammals, bovines, rats, mice, dogs, monkeys, goat, sheep, cows, deer, and other non mammalian animals. In embodiments, a patient is human.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer, prostate cancer, pancreatic cancer, brain cancer, liver cancer, gastric cancer or a sarcoma.

In embodiments, the disease is an inflammatory disease. As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

In embodiments, the disease is a neurodegenerative disease. As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

The schematic provided depicts a block diagram illustrating an example of a computing system 500, controller, or PC, in accordance with some example embodiments. Referring to the schematic above, the computing system 500 can be used to process data, process images, control hardware, synchronize processing hardware control and/or any steps useful for the methods provided herein.

As shown in the schematic above, the computing system 500 can include a processor 510, a memory 520, a storage device 530, and an input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of, for example, the logging controller. In some implementations of the current subject matter, the processor 510 can be a single-threaded processor. Alternately, the processor 510 can be a multi-threaded processor. The processor 510 is capable of processing instructions stored in the memory 520 and/or on the storage device 530 to display graphical information for a user interface or hardware sensing or control provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an integrated circuit device, an optical disk device, or a tape device, or other suitable persistent storage means. The input/output device 540 provides input/output operations for the computing system 500 and hardware sensing or control. In some implementations of the current subject matter, the input/output device 540 includes a keyboard and/or pointing device. In various implementations, the input/output device 540 includes a display unit for displaying graphical user interfaces. In some implementations of the current subject matter, device 540 includes a collection of input/output devices. In some implementations of the current subject matter, device 540 includes parts of a microscopy system. In some implementations of the current subject matter, device 540 includes parts of an irradiation unit, a graphics processing unit, a DMD, and the DMD Controller.

According to some implementations of the current subject matter, the input/output device 540 can provide input/output operations for a network device or a device connected through serial, parallel, synchronous, or asynchronous connections. For example, the input/output device 540 can include Ethernet ports or other networking ports to communicate with one or more wired and/or wireless networks (e.g., a local area network (LAN), a wide area network (WAN), the Internet). For example, the input/output device 540 can include Serial RS-232, or RS-485 or other interfaces to communicate with another computing system 500. According to some implementations of the current subject matter, the computing system 500 includes multiple computing systems 500 units connected and communicating through input/output devices 540 or similar devices.

In some implementations of the current subject matter, the computing system 500 can be used to execute various interactive computer software applications that can be used for organization, analysis and/or storage of data in various (e.g., tabular) format (e.g., Microsoft Excel®, and/or any other type of software). Alternatively, the computing system 500 can be used to execute any type of software applications. These applications can be used to perform various functionalities, e.g., scheduling and timing functionalities (e.g., generating, managing, editing of event schedules, processing commands and/or hardware sensing and control, etc.), computing functionalities (e.g., processing of data, images or signals), communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

### Methods

Provided herein are methods for, inter alia, separating and/or quantitating subpopulations of cells from a population of cells using simultaneous tagging methods. The methods provided are, inter alia, useful for analyzing, modulating and utilizing selective cell populations to study diseases, advance drug discovery and identify novel treatment pathways.

In an aspect, a method of irradiating a selected sub-population of cells within a population of cells is provided. The method includes: a) simultaneously irradiating each of a first selected sub-population of cells within a population of cells within a first digital image of a first microscope field of view with a first dose of light, thereby forming a first irradiated sub-population of cells and a remainder of cells within the population of cells. At least a portion of the remainder of cells within the population of cells are labeled with the same photosensitive label as the first selected sub-population of cells. A portion of the first selected sub-population of cells includes a first cellular phenotype not present in a portion of the remainder of cells within the population of cell. And b) quantitating the first irradiated sub-population of cells or separating the first irradiated sub-population of cells from the remainder of cells.

“A selected sub-population of cells” as provided herein refers to a plurality of cells (e.g., more than one cell, at least two cells) wherein at least a portion of the cells have a common cellular phenotype of interest. Thus, in embodiments, a portion of the selected subpopulation of cells shares a common cellular phenotype (e.g., a first cellular phenotype, a second cellular phenotype).

A “cellular phenotype” as provided herein refers to a characteristic (e.g., inherent characteristic) of physical cells useful for the methods provided herein in selecting a subpopulation of cells within a population of cells. A cellular phenotype as provided herein may include one or more characteristics (e.g., a first cellular phenotype, a second cellular phenotype, a third cellular phenotype, or a fourth cellular phenotype) of one or more physical cells (e.g., a first cellular phenotype, a second cellular phenotype, a third cellular phenotype, or a fourth cellular phenotype). The one or more characteristics (e.g., a first cellular phenotype, a second cellular phenotype, a third cellular phenotype, or a fourth cellular phenotype) of physical cells may change over time and/relative to a standard control. Thus, in embodiments, the cellular phenotype (e.g., a first cellular phenotype, a second cellular phenotype, a third cellular phenotype, or a fourth cellular phenotype) includes changes of the one or more characteristics or their locations over time. Examples of a cellular phenotype include, without limitation, physical interaction of a cell with another cell, physical interaction of a cell with its environment (e.g., cell culture medium, growth factors, solid supports of a culturing vessel), expression or activity (including level of expression or level of activity, location of expression, localization) of one or more proteins (membrane protein, cell surface protein, intracellular (nuclear, cytoplasmic) protein), expression or activity (including level of expression, location of expression, localization) of one or more genes (including expression patterns), expression (including level of expression, location of expression, localization) of one or more RNA molecules (including RNA transcripts with or without specific post-transcription modifications, mRNA, siRNA, microRNA), an activity level of one or more gene promoters, enhancers, or silencers, one or more morphologically or fluorometrically distinguishing features, a physical location, condition or count of one or more organelles or structures associated with the cell, a physical location of one or more cells within a tissue, a physical location of one or more cells within an in vitro cell culture, a physical location and its change over time of one or more cells relative to one another or to an in vitro cell culture or tissue, interactions with one or more cells, objects or constituents.

In embodiments, the cellular phenotype includes the presence or absence of one or more epigenetic markers, one or more post-translational modifications (e.g., glycosylation), one or more organelles, subcellular structures, differentiation or proliferation events, or apoptosis, autophagy, necrosis or entosis. In embodiments, the cellular phenotype includes the presence or absence of interactions between a cell and external constituents (e.g., one or more other cells or non-cellular objects such as beads), including formation of membrane junctions or synapses, interactions mediated by receptors, interactions mediated by signaling mechanisms (autocrine, paracrine, or endocrine), interactions mediated by ion channels, interactions mediated by endocytosis, exocytosis, pinocytosis, or phagocytosis, interactions mediated by surface biomolecules, interactions mediated by antibodies). In embodiments, the cellular phenotype includes the expression level or location of markers for organic or inorganic molecules (e.g., inorganic ions, RNA, DNA, lipid, amino acids, peptides, polypeptides, and proteins). In embodiments, the cellular phenotype includes pH, membrane potential, relative prevalence of ions, morphological characteristics, stress, age, or temperature and their change over time. In embodiments, the cellular phenotype includes biomechanical activity including deformations, strain, stress.

For the methods provided herein microscopy methods may be used for the assessment of, for example, a cellular phenotype. Non-limiting examples of microscopy techniques useful for the methods provided herein including embodiments thereof include, wide field microscopy bright field microscopy, phase contrast microscopy, differential interference contrast microscopy, single- or multi-photon fluorescence microscopy, fluorescence microscopy, photoacoustic microscopy, luminescence microscopy, Raman scattering microscopy, two-dimensional microscopy, or three-dimensional microscopy. In embodiments, the microscopy techniques utilize transmitted illumination, bright field illumination, epi-illumination, dark field illumination, wide field illumination, point-scanning illumination, line-scanning illumination, spinning disk illumination, speckled illumination, or patterned illumination.

In embodiments, the sub-population of cells forms part of an in vitro cell culture. In embodiments, the sub-population of cells forms part of a mono-layer. In embodiments, the sub-population are adherent cells. In embodiments, the sub-population are non-adherent cells. In embodiments, the sub-population of cells forms part of an organoid, tumoroid, or spheroid. In embodiments, the sub-population of cells forms part of a tissue. In embodiments, sub-population of cells forms part of a biological sample. In embodiments, the biological sample is derived from a subject. In embodiments, the subject is a mammal. In embodiments, the subject is human. In embodiments, the subject is a patient. In embodiments, the subject is a patient undergoing treatment for a disease. In embodiments, the subject is a subject that has or is at risk of having a disease. In embodiments, the disease is cancer. In embodiments, the disease is a neurological disease. In embodiments, the disease is diabetes. In embodiments, the sub-population of cells includes one or more cells expressing one or more recombinant proteins or nucleic acids. In embodiments, the recombinant protein is CRISPR. In embodiments, the recombinant nucleic acid encodes a CRISPR protein. In embodiments, the recombinant nucleic acid encodes a guide RNA.

For the methods provided herein a cellular phenotype (e.g., first cellular phenotype, second cellular phenotype) is selected for conducting the methods provided herein prior to the simultaneously irradiating each of a first selected sub-population of cells within a population of cells. In embodiments, the cellular phenotype (e.g., first cellular phenotype, second cellular phenotype) is not selected after the simultaneously irradiating each of a first selected sub-population of cells within a population of cells. In embodiments, the cellular phenotype (e.g., first cellular phenotype, second cellular phenotype) is selected after the simultaneously irradiating each of a first selected sub-population of cells within a population of cells.

In embodiments, the cellular phenotype (e.g., first cellular phenotype, second cellular phenotype) does not include a physical location of a portion of the selected subpopulation of cells within a tissue. In embodiments, the cellular phenotype (e.g., first cellular phenotype, second cellular phenotype) does not include a physical location of each of the selected subpopulation of cells within a tissue.

In embodiments, the cellular phenotype (e.g., first cellular phenotype, second cellular phenotype) does not include a physical location of a portion of the selected subpopulation of cells within a organoid or spheroid. In embodiments, the cellular phenotype (e.g., first cellular phenotype, second cellular phenotype) does not include a physical location of each of the selected subpopulation of cells within a spheroid.

In embodiments, the cellular phenotype (e.g., first cellular phenotype, second cellular phenotype) does not include a physical location of a portion of the selected subpopulation of cells within an organ. In embodiments, the cellular phenotype (e.g., first cellular phenotype, second cellular phenotype) does not include a physical location of each of the selected subpopulation of cells within an organ.

In embodiments, the selected subpopulation of cells does not form part of a tissue sample from an organism. In embodiments, the selected subpopulation of cells does not form part of a spheroid. In embodiments, the selected subpopulation of cells does not form part of a tissue.

In embodiments, a portion of the selected subpopulation of cells does not form part of a tissue sample from an organism. In embodiments, a portion of the selected subpopulation of cells does not form part of a spheroid. In embodiments, a portion of the selected subpopulation of cells does not form part of a tissue.

In embodiments, all of the cells having a common phenotype of interest in the first field of view are selected. In embodiments, at least 90% of the cells having a common phenotype of interest in the first field of view are selected. In embodiments, at least 80% of the cells having a common phenotype of interest in the first field of view are selected. In embodiments, at least 70% of the cells having a common phenotype of interest in the first field of view are selected. In embodiments, at least 60% of the cells having a common phenotype of interest in the first field of view are selected. In embodiments, at least 50% of the cells having a common phenotype of interest in the first field of view are selected. In embodiments, at least 40% of the cells having a common phenotype of interest in the first field of view are selected. In embodiments, at least 30% of the cells having a common phenotype of interest in the first field of view are selected. In embodiments, at least 20% of the cells having a common phenotype of interest in the first field of view are selected. In embodiments, at least 10% of the cells having a common phenotype of interest in the first field of view are selected. In embodiments, at least 5% of the cells having a common phenotype of interest in the first field of view are selected. In embodiments, at least 1% of the cells having a common phenotype of interest in the first field of view are selected.

In embodiments, about 90% of the cells having a common phenotype of interest in the first field of view are selected. In embodiments, about 80% of the cells having a common phenotype of interest in the first field of view are selected. In embodiments, about 70% of the cells having a common phenotype of interest in the first field of view are selected. In embodiments, about 60% of the cells having a common phenotype of interest in the first field of view are selected. In embodiments, about 50% of the cells having a common phenotype of interest in the first field of view are selected. In embodiments, about 40% of the cells having a common phenotype of interest in the first field of view are selected. In embodiments, about 30% of the cells having a common phenotype of interest in the first field of view are selected. In embodiments, about 20% of the cells having a common phenotype of interest in the first field of view are selected. In embodiments, about 10% of the cells having a common phenotype of interest in the first field of view are selected. In embodiments, about 5% of the cells having a common phenotype of interest in the first field of view are selected. In embodiments, about 1% of the cells having a common phenotype of interest in the first field of view are selected.

In embodiments, 90%-100% of the cells having a common phenotype of interest in the first field of view are selected. In embodiments, 80%-100% of the cells having a common phenotype of interest in the first field of view are selected. In embodiments, 70%-100% of the cells having a common phenotype of interest in the first field of view are selected. In embodiments, 60%-100% of the cells having a common phenotype of interest in the first field of view are selected. In embodiments, 50%-100% of the cells having a common phenotype of interest in the first field of view are selected. In embodiments, 40%-100% of the cells having a common phenotype of interest in the first field of view are selected. In embodiments, 30%-100% of the cells having a common phenotype of interest in the first field of view are selected. In embodiments, 20%-100% of the cells having a common phenotype of interest in the first field of view are selected. In embodiments, 10%-100% of the cells having a common phenotype of interest in the first field of view are selected. In embodiments, 5%-100% of the cells having a common phenotype of interest in the first field of view are selected. In embodiments, 1%-100% of the cells having a common phenotype of interest in the first field of view are selected.

In embodiments, 0.1%-5% of the cells having a common phenotype of interest in the first field of view are selected. In embodiments, 0.1%-10% of the cells having a common phenotype of interest in the first field of view are selected. In embodiments, 0.1%-15% of the cells having a common phenotype of interest in the first field of view are selected. In embodiments, 0.1%-20% of the cells having a common phenotype of interest in the first field of view are selected. In embodiments, 0.1%-30% of the cells having a common phenotype of interest in the first field of view are selected. In embodiments, 0.1%-40% of the cells having a common phenotype of interest in the first field of view are selected. In embodiments, 0.1%-50% of the cells having a common phenotype of interest in the first field of view are selected. In embodiments, 0.1%-60% of the cells having a common phenotype of interest in the first field of view are selected. In embodiments, 0.1%-70% of the cells having a common phenotype of interest in the first field of view are selected. In embodiments, 0.1%-80% of the cells having a common phenotype of interest in the first field of view are selected. In embodiments, 0.1%-90% of the cells having a common phenotype of interest in the first field of view are selected.

In embodiments, about 0.1%, 1%, 2%, 5%, 10%, 20%, 30%, 40%, 50%, 60%, 70%, 80%, 90%, 95%, 99% or 100% of the cells having a common phenotype of interest in the first field of view are selected. In embodiments, 0.1%, 1%, 2%, 5%, 10%, 20%, 30%, 40%, 50%, 60%, 70%, 80%, 90%, 95%, 99% or 100% of the cells having a common phenotype of interest in the first field of view are selected.

The term “irradiating” is used herein in its customary sense known in the art and refers to administering light at a specific dose, a specific intensity and/or for a specific length of time to a cell of the sub-population of cells as provided herein. Likewise, “simultaneously irradiating” as provided herein refers to administering light to the cells of a selected sub-population of cells (e.g., first, second, third, fourth sub-population of cells) as provided herein at the same time. The dose, intensity, and duration of irradiation may each be different or the same when the cells of a selected subpopulation are simultaneously irradiated. A “dose of light” as provided herein refers to the sum of energy of light administered over a certain duration of time to the selected sub-population of cells provided herein. The phrase “intensity of light” as provided herein is an amount of light administered to a standard unit of area in a standard unit of time. The dose of light in a given area may be equal to the intensity of light in that area multiplied by the duration of the irradiation.

In embodiments, the population of cells includes a second selected sub-population of cells and each of the second selected sub-population of cells within the population of cells is simultaneously irradiated with a second dose of light, thereby forming a second irradiated sub-population of cells. As described above, light is administered to each of the second selected sub-population of cells at the same time, while the dose, intensity, and duration of irradiation may each be different or the same.

The dose of light administered to irradiate the first selected sub-population of cells (e.g., first dose of light) may the same as or different from the dose of light administered to irradiate the second selected sub-population of cells (e.g., second dose of light). Thus, in embodiments, the first dose of light and the second dose of light are the same or different. In embodiments, the first dose of light and the second dose of light are the same. In embodiments, the first dose of light and the second dose of light are different.

In embodiments, the first selected sub-population of cells and the second selected sub-population of cells are simultaneously irradiated. In embodiments, the first dose of light corresponds to a first length of irradiation time for which the first selected sub-population of cells is irradiated, and the second dose of light corresponds to a second length of irradiation time for which the second selected sub-population of cells is irradiated. In embodiments, the first length of irradiation time and the second length of irradiation time are the same or different. In embodiments, the first length of irradiation time and the second length of irradiation time are the same. In embodiments, the first length of irradiation time and the second length of irradiation time are different.

In embodiments, the first length of irradiation time is shorter or longer than the second length of irradiation time. In embodiments, the first length of irradiation time is shorter than the second length of irradiation time. In embodiments, the first length of irradiation time is longer than the second length of irradiation time. In embodiments, the first length of irradiation time and the second length of irradiation time start at the same timepoint or at different timepoints. In embodiments, the first length of irradiation time and the second length of irradiation time start at the same timepoint. In embodiments, the first length of irradiation time and the second length of irradiation time start at different timepoints. In embodiments, the first length of irradiation time and the second length of irradiation time end at the same timepoint or at different timepoints. In embodiments, the first length of irradiation time and the second length of irradiation time end at the same timepoint. In embodiments, the first length of irradiation time and the second length of irradiation time end at different timepoints.

In embodiments, the first length of irradiation time starts before or after the second length of irradiation time. In embodiments, the first length of irradiation time starts before the second length of irradiation time. In embodiments, the first length of irradiation time starts after the second length of irradiation time.

In embodiments, the first dose of light corresponds to a first intensity of light at which the first selected sub-population of cells is irradiated, and the second dose of light corresponds to a second intensity of light at which the second selected sub-population of cells is irradiated.

In embodiments, the first intensity of light and the second intensity of light are the same or different. In embodiments, the first intensity of light and the second intensity of light are the same. In embodiments, the first intensity of light and the second intensity of light are different.

In embodiments, the first dose of light and the second dose of light correspond to a duty cycle of an irradiation unit irradiating the first selected sub-population of cells and the second selected sub-population of cells. In embodiments, the irradiation unit includes a light source. In embodiments, the light source includes one or more of a light emitting diode (LED), a laser, an arc lamp, or an incandescent lamp. In embodiments, the irradiation unit further includes a light patterning mechanism. In embodiments, the light patterning mechanism includes one or more of a digital micromirror device (DMD). In embodiments, the light patterning mechanism includes a spatial light modulator. In embodiments, the light patterning mechanism includes a deformable mirror. In embodiments, the light patterning mechanism includes a liquid crystal display (LCD). In embodiments, the light patterning mechanism includes a galvanometer scanner. In embodiments, the light patterning mechanism includes an acousto-optic deflector (AOD). In embodiments, the light patterning mechanism includes a spinning disk. In embodiments, the light patterning mechanism includes a multiphoton microscopy module.

In embodiments, at least 99% of the remainder of cells are not irradiated at the first dose of light at the time each of the first selected sub-population of cells is irradiated at the first dose of light. In embodiments, about 99% of the remainder of cells are not irradiated at the first dose of light at the time each of the first selected sub-population of cells is irradiated at the first dose of light. In embodiments, 100% of the remainder of cells are not irradiated at the first dose of light at the time each of the first selected sub-population of cells is irradiated at the first dose of light.

In embodiments, at least 99% of the remainder of cells are not irradiated at the second dose of light at the time each of the second selected sub-population of cells is irradiated at the second dose of light. In embodiments, about 99% of the remainder of cells are not irradiated at the second dose of light at the time each of the second selected sub-population of cells is irradiated at the second dose of light. In embodiments, 100% of the remainder of cells are not irradiated at the second dose of light at the time each of the second selected sub-population of cells is irradiated at the second dose of light.

In embodiments, the population of cells includes an additional (second, third, fourth, fifth, sixth, seventh etc.) selected sub-population of cells and each cell of the additional (second, third, fourth, fifth, sixth, seventh etc.) selected sub-population of cells within the population of cells is simultaneously irradiated at an additional (second, third, fourth, fifth, sixth, seventh etc.) dose of light, thereby forming an additional irradiated sub-population of cells. Any embodiments, described herein for the first and second selected sub-population of cells are applicable to the additional (third, fourth, fifth, sixth, seventh etc.) selected sub-population of cells. A person of ordinary skill in the art will immediately recognize that the methods provided herein are not limited to one or two selected sub-populations of cells, but may be performed on a plurality of selected sub-populations of cells each exhibiting, for example, distinct cellular phenotypes. Thus, where an additional selected sub-population of cells is present, the first dose of light and the additional dose of light may be the same or different. In embodiments, the first dose of light and the additional dose of light are the same. In embodiments, the first dose of light and the additional dose of light are different.

For the methods provided a cell of a subpopulation of cells as provided herein may be tagged for several distinct phenotypes. Thus, a cell of a subpopulation of cells as provided herein may form part of a first, second or third subpopulation of cells during the process. In embodiments, a cell of a subpopulation of cells forms part of the first and the second subpopulation of cells. In embodiments, a cell of a subpopulation of cells does not form part of the first subpopulation of cells and forms part of the second subpopulation of cells. In embodiments, a cell of a subpopulation of cells forms part of the first subpopulation of cells and does not form part of the second subpopulation of cells. In embodiments, a cell of a subpopulation of cells forms part of the first and the additional subpopulation of cells. In embodiments, a cell of a subpopulation of cells does not form part of the first subpopulation of cells and forms part of the additional subpopulation of cells. In embodiments, a cell of a subpopulation of cells forms part of the first subpopulation of cells and does not form part of the additional subpopulation of cells.

In embodiments, the first selected sub-population of cells and the additional selected sub-population of cells are simultaneously irradiated. In embodiments, the first selected sub-population of cells is simultaneously irradiated at a starting timepoint t1. In embodiments, the additional selected sub-population of cells is simultaneously irradiated at an additional starting timepoint t. In embodiments, the t1 and the t are the same. In embodiments, the t1 precedes the t. In embodiments, the first selected sub-population of cells is simultaneously irradiated for a first length of irradiation time. In embodiments, the first length of irradiation time ends at an endpoint tf1. In embodiments, the additional selected sub-population of cells is simultaneously irradiated for an additional length of irradiation time. In embodiments, the additional length of irradiation time ends at an endpoint tf. In embodiments, the endpoint tf1 and the endpoint tf are the same or different. In embodiments, the first length of irradiation time and the additional length of irradiation time are the same. In embodiments, the first length of irradiation time and the additional length of irradiation time are different. In embodiments, the first length of irradiation time is shorter or longer relative to the additional length of irradiation time.

In embodiments, the simultaneous irradiating is based on the location of the first selected sub-population of cells within the first digital image of a first microscope field of view. In embodiments, the simultaneous irradiating is further based on the location of the first selected sub-population of cells within a plurality of first digital images of a first microscope field of view.

In embodiments, the simultaneous irradiating is based on the location of the second selected sub-population of cells within the first digital image of a first microscope field of view. In embodiments, the simultaneous irradiating is further based on the location of the second selected sub-population of cells within a plurality of digital images of a first microscope field of view.

The term “digital image” as provided herein refers to a digital image which includes specific pixel values and irradiation values required for the step for irradiating a selected sub-population of cells within a population of cells. In a digital image provided herein each pixel corresponds to a micromirror in the DMD and each pixel value is set proportional or equal to the dose of light (first, second or additional dose of light) administered during the irradiation step. The digital image may be derived from a raw projection image. A raw projection image is a digital image including information on the pixel value and dose of light used for irradiation (irradiation value). In embodiments, the first digital image is formed from a first raw projection image. The raw projection image may further be processed by transformation of the pixel coordinates through processes of, for example, cropping, and/or geometric transformation thereby forming a digital image. Transformation as provided herein includes calculating the difference between a recorded position where a cell image is acquired and the position where the digital image (e.g., projection image) is projected or will be projected to irradiate the cells. The raw projection image may be derived from a raw digital image. In a raw digital image each cell within the population of cells is mapped to one or more corresponding pixels. The mapping may be performed by using a lookup table. In embodiments, the digital image is a raw digital image. In embodiments, a raw digital image includes pixels, wherein each pixel corresponds to a pixel in a camera and each pixel value is proportional to the amount of light acquired at that pixel or calculated from the pixel or pixels values therein.

In embodiments, the method further includes: selecting, based at least on a lookup table (LUT), the first selected sub-population of cells, the lookup table mapping each cell within the population of cells to one or more corresponding pixels in a first raw digital image, the pixels corresponding to the first selected sub-population of cells including a subset lookup table (LUT), and the first raw projection image being formed by assigning a desired irradiation value to each pixel included in the subset lookup table.

In embodiments, the first raw projection image is formed from the first raw digital image. In embodiments, the first raw projection image further includes at least a portion of an additional microscope field of view. In embodiments, the second raw projection image is formed from a second raw digital image. In embodiments, the second raw projection image further includes at least a portion of an additional microscope field of view. Where a projection image includes a portion of an additional microscope field of view, the image is referred to herein as “stitched projection image” or “stitched digital image.” In embodiments, the second digital image is formed from a second raw projection image. In embodiments, the first digital image includes at least a portion of a second microscope field of view. In embodiments, the simultaneous irradiating is further based on the location of the first selected sub-population of cells within a second digital image of a second microscope field of view. In embodiments, the simultaneous irradiating is further based on the location of the second selected sub-population of cells within a second digital image of a second microscope field of view.

In embodiments, the population of cells is included in a sample and the sample moves with a movement velocity. In embodiments, the method further includes: determining, based at least on the movement velocity of the sample, one or more transformations for forming the first digital image or the second digital image; and applying the one or more transformations to form the first digital image or the second digital image. In embodiments, the one or more transformations include cropping. In embodiments, the one or more transformations include a geometric transformation. In embodiments, the geometric transformation includes one or more of a Euclidean transformation, an affine transformation, or a projective transformation. In embodiments, the movement velocity is predetermined.

In embodiments, the method further includes: determining the movement velocity of the sample at a first time and a second time; determining, based at least on a first movement velocity of the sample at the first time, a first transformation for forming the first digital image; and determining, based at least on a second movement velocity of the sample at the second time, a second transformation for forming the second digital image.

In embodiments, the sample is in an irradiation device.

In embodiments, a portion of the second selected sub-population of cells includes a second cellular phenotype not present in a portion of the remainder of cells within the population of cells. In embodiments, the method further includes quantitating the second irradiated sub-population of cells or separating the second irradiated sub-population of cells from the remainder of cells. In embodiments, the first cellular phenotype and the second cellular phenotype are different or the same. In embodiments, the first cellular phenotype and the second cellular phenotype are different. In embodiments, the first cellular phenotype and the second cellular phenotype are the same.

In embodiments, the portion of the first selected sub-population of cells including a first cellular phenotype is at least 0.1% of the first selected sub-population of cells. In embodiments, the portion of the first selected sub-population of cells including a first cellular phenotype is at least 1% of the first selected sub-population of cells. In embodiments, the portion of the first selected sub-population of cells including a first cellular phenotype is at least 5% of the first selected sub-population of cells. In embodiments, the portion of the first selected sub-population of cells including a first cellular phenotype is at least 10% of the first selected sub-population of cells. In embodiments, the portion of the first selected sub-population of cells including a first cellular phenotype is at least 20% of the first selected sub-population of cells. In embodiments, the portion of the first selected sub-population of cells including a first cellular phenotype is at least 30% of the first selected sub-population of cells. In embodiments, the portion of the first selected sub-population of cells including a first cellular phenotype is at least 40% of the first selected sub-population of cells. In embodiments, the portion of the first selected sub-population of cells including a first cellular phenotype is at least 50% of the first selected sub-population of cells. In embodiments, the portion of the first selected sub-population of cells including a first cellular phenotype is at least 60% of the first selected sub-population of cells. In embodiments, the portion of the first selected sub-population of cells including a first cellular phenotype is at least 70% of the first selected sub-population of cells. In embodiments, the portion of the first selected sub-population of cells including a first cellular phenotype is at least 80% of the first selected sub-population of cells. In embodiments, the portion of the first selected sub-population of cells including a first cellular phenotype is at least 90% of the first selected sub-population of cells. In embodiments, the portion of the first selected sub-population of cells including a first cellular phenotype is at least 99% of the first selected sub-population of cells.

In embodiments, the portion of the first selected sub-population of cells including a first cellular phenotype is about 0.1% of the first selected sub-population of cells. In embodiments, the portion of the first selected sub-population of cells including a first cellular phenotype is about 1% of the first selected sub-population of cells. In embodiments, the portion of the first selected sub-population of cells including a first cellular phenotype is about 5% of the first selected sub-population of cells. In embodiments, the portion of the first selected sub-population of cells including a first cellular phenotype is about 10% of the first selected sub-population of cells. In embodiments, the portion of the first selected sub-population of cells including a first cellular phenotype is about 20% of the first selected sub-population of cells. In embodiments, the portion of the first selected sub-population of cells including a first cellular phenotype is about 30% of the first selected sub-population of cells. In embodiments, the portion of the first selected sub-population of cells including a first cellular phenotype is about 40% of the first selected sub-population of cells. In embodiments, the portion of the first selected sub-population of cells including a first cellular phenotype is about 50% of the first selected sub-population of cells. In embodiments, the portion of the first selected sub-population of cells including a first cellular phenotype is about 60% of the first selected sub-population of cells. In embodiments, the portion of the first selected sub-population of cells including a first cellular phenotype is about 70% of the first selected sub-population of cells. In embodiments, the portion of the first selected sub-population of cells including a first cellular phenotype is about 80% of the first selected sub-population of cells. In embodiments, the portion of the first selected sub-population of cells including a first cellular phenotype is about 90% of the first selected sub-population of cells. In embodiments, the portion of the first selected sub-population of cells including a first cellular phenotype is about 99% of the first selected sub-population of cells.

In embodiments, the portion of the second selected sub-population of cells including a second cellular phenotype is at least 0.1% of the second selected sub-population of cells. In embodiments, the portion of the second selected sub-population of cells including a second cellular phenotype is at least 1% of the second selected sub-population of cells. In embodiments, the portion of the second selected sub-population of cells including a second cellular phenotype is at least 5% of the second selected sub-population of cells. In embodiments, the portion of the second selected sub-population of cells including a second cellular phenotype is at least 10% of the second selected sub-population of cells. In embodiments, the portion of the second selected sub-population of cells including a second cellular phenotype is at least 20% of the second selected sub-population of cells. In embodiments, the portion of the second selected sub-population of cells including a second cellular phenotype is at least 30% of the second selected sub-population of cells. In embodiments, the portion of the second selected sub-population of cells including a second cellular phenotype is at least 40% of the second selected sub-population of cells. In embodiments, the portion of the second selected sub-population of cells including a second cellular phenotype is at least 50% of the second selected sub-population of cells. In embodiments, the portion of the second selected sub-population of cells including a second cellular phenotype is at least 60% of the second selected sub-population of cells. In embodiments, the portion of the second selected sub-population of cells including a second cellular phenotype is at least 70% of the second selected sub-population of cells. In embodiments, the portion of the second selected sub-population of cells including a second cellular phenotype is at least 80% of the second selected sub-population of cells. In embodiments, the portion of the second selected sub-population of cells including a second cellular phenotype is at least 90% of the second selected sub-population of cells. In embodiments, the portion of the second selected sub-population of cells including a second cellular phenotype is at least 99% of the second selected sub-population of cells.

In embodiments, the portion of the second selected sub-population of cells including a second cellular phenotype is about 0.1% of the second selected sub-population of cells. In embodiments, the portion of the second selected sub-population of cells including a second cellular phenotype is about 1% of the second selected sub-population of cells. In embodiments, the portion of the second selected sub-population of cells including a second cellular phenotype is about 5% of the second selected sub-population of cells. In embodiments, the portion of the second selected sub-population of cells including a second cellular phenotype is about 10% of the second selected sub-population of cells. In embodiments, the portion of the second selected sub-population of cells including a second cellular phenotype is about 20% of the second selected sub-population of cells. In embodiments, the portion of the second selected sub-population of cells including a second cellular phenotype is about 30% of the second selected sub-population of cells. In embodiments, the portion of the second selected sub-population of cells including a second cellular phenotype is about 40% of the second selected sub-population of cells. In embodiments, the portion of the second selected sub-population of cells including a second cellular phenotype is about 50% of the second selected sub-population of cells. In embodiments, the portion of the second selected sub-population of cells including a second cellular phenotype is about 60% of the second selected sub-population of cells. In embodiments, the portion of the second selected sub-population of cells including a second cellular phenotype is about 70% of the second selected sub-population of cells. In embodiments, the portion of the second selected sub-population of cells including a second cellular phenotype is about 80% of the second selected sub-population of cells. In embodiments, the portion of the second selected sub-population of cells including a second cellular phenotype is about 90% of the second selected sub-population of cells. In embodiments, the portion of the second selected sub-population of cells including a second cellular phenotype is about 99% of the second selected sub-population of cells.

In embodiments, the portion of the remainder of cells within the population of cells wherein the first cellular phenotype is not present is 50% of the remainder of cells. In embodiments, the portion of the remainder of cells within the population of cells wherein the first cellular phenotype is not present is at least 90% of the remainder of cells. In embodiments, the portion of the remainder of cells within the population of cells wherein the first cellular phenotype is not present is at least 99% of the remainder of cells.

In embodiments, the portion of the remainder of cells within the population of cells wherein the second cellular phenotype is not present is 50% of the remainder of cells. In embodiments, the portion of the remainder of cells within the population of cells wherein the second cellular phenotype is not present is at least 90% of the remainder of cells. In embodiments, the portion of the remainder of cells within the population of cells wherein the second cellular phenotype is not present is at least 99% of the remainder of cells.

In embodiments, at least a portion of the remainder of cells within the population of cells are labeled with the same photosensitive label as the second selected sub-population of cells.

In embodiments, the first selected sub-population of cells and the second selected sub-population of cells are labeled with the same photosensitive label.

In embodiments, the first selected sub-population of cells is labeled with a first photosensitive label and the second selected sub-population of cells is labeled with a second photosensitive label.

In embodiments, the portion of the remainder of cells within the population of cells labeled with the same photosensitive label is at least 50% of the remainder of cells. In embodiments, the portion of the remainder of cells within the population of cells labeled with the same photosensitive label is at least 90% of the remainder of cells. In embodiments, the portion of the remainder of cells within the population of cells labeled with the same photosensitive label is at least 99% of the remainder of cells. In embodiments, the portion of the remainder of cells within the population of cells labeled with the same photosensitive label is 100% of the remainder of cells.

In embodiments, a portion of the remainder of cells within the population of cells are not labeled with the same photosensitive label as the first selected sub-population of cells. In embodiments, a portion of the remainder of cells within the population of cells are unlabeled.

In embodiments, the simultaneously irradiating activates the photosensitive label. In embodiments, the simultaneous irradiation deactivates the photosensitive label.

In embodiments, the photosensitive label is attached to the first selected sub-population of cells or the remainder of cells through a chemical linker. In embodiments, the chemical linker is a covalent linker or a non-covalent linker. In embodiments, the chemical linker includes a nucleic acid. In embodiments, the chemical linker includes a lipid, cholesterol, a phospholipid, a fatty-acid, an amphiphile, polycations, polyanions, or polyethyleneglycol (PEG) molecules.

In embodiments, the chemical linker includes a double-stranded nucleic acid. In embodiments, the chemical linker includes a unique molecular identifier (UMI).

In embodiments, the non-covalent linker includes an antibody. In embodiments, the non-covalent linker includes an antibody-nucleic acid conjugate. In embodiments, the photosensitive label is a labeling oligonucleotide including a photosensitive blocking moiety. In embodiments, the labeling oligonucleotide includes a unique molecular identifier (UMI). In embodiments the labeling oligonucleotide includes an alkyl chain, fatty acid chain, cholesterol moiety, phosphorylated fatty acid chain, or other hydrophobic subunit.

In embodiments, the simultaneous irradiation in a) further includes deprotecting the labeling oligonucleotide thereby removing the photosensitive blocking moiety from the labeling oligonucleotide and forming a deprotected labeling oligonucleotide. In embodiments, the quantitating in b) further includes: i) contacting the deprotected labeling oligonucleotide with a template oligonucleotide and a polymerase or a ligase their required cofactors and thereby forming a barcoded oligonucleotide. And ii) detecting the barcoded oligonucleotide.

In embodiments, the photosensitive label is a labeling oligonucleotide including a plurality of photosensitive blocking moieties each attached to a nucleotide of the labeling oligonucleotide.

In embodiments, the simultaneous irradiation in a) further includes deprotecting the labeling oligonucleotide thereby removing the plurality of photosensitive blocking moieties from the labeling oligonucleotide and forming a deprotected labeling oligonucleotide. In embodiments, the quantitating in b) further includes: i) contacting the deprotected labeling oligonucleotide with a template oligonucleotide and a polymerase or a ligase and any required cofactors thereby forming a barcoded oligonucleotide. And ii) detecting the barcoded oligonucleotide.

In embodiments, the photosensitive label includes a fluorophore moiety. In embodiments, the fluorophore moiety is an organic dye. In embodiments, the fluorophore moiety is an inorganic dye. In embodiments, the fluorophore moiety is a fluorescent protein. In embodiments, the fluorophore moiety is a photocaged molecule. In embodiments the fluorescent protein can be photoactivated.

In embodiments, the photosensitive label includes one or more of a photolabile protecting groups. In embodiments, the photosensitive label includes a template oligonucleotide attached to a fluorophore moiety. In embodiments, the fluorophore moiety is a fluorescent protein.

In embodiments, the photosensitive label includes a template oligonucleotide hybridized to a labeling oligonucleotide, wherein the labeling oligonucleotide is attached to a fluorophore moiety. In embodiments, the fluorophore moiety is a fluorescent protein.

In embodiments, the photosensitive label includes a template oligonucleotide hybridized to a labeling oligonucleotide, wherein the template oligonucleotide is attached to a fluorophore moiety. In embodiments, the fluorophore moiety is a fluorescent protein.

In an aspect, a method of selecting a sub-population of cells within a population of cells is provided. The method includes: a) simultaneously irradiating each of a first selected sub-population of cells within a population of cells within a first digital image of a first microscope field of view with a first dose of light, thereby forming a first non-irradiated sub-population of cells and a remainder of cells within the population of cells. At least a portion of the remainder of cells within the population of cells are labeled with the same photosensitive label as the first selected sub-population of cells. A portion of the first selected sub-population of cells includes a first cellular phenotype not present in a portion of the remainder of cells within the population of cells. And b) quantitating or characterizing the non-irradiated sub-population of cells or separating the non-irradiated sub-population of cells from the remainder of cells.

In embodiments, quantitating, characterizing, or separating cells includes subjecting cells to image cytometry, flow cytometry, or cell sorting via FACS, single-cell dispensing, or magnetic sorting. In embodiments, the cells' RNA or DNA is sequenced. In embodiments, the cells are subjected to proteomics. In embodiments, the sequencing or proteomics is performed on a single cell application. In embodiments, the quantitated, characterized, or separated cells are used in in vitro testing. In embodiments, the quantitated, characterized, or separated cells are further cultured. In embodiments, the quantitated, characterized, or separated cells are engineered. In embodiments, the quantitated, characterized, or separated cells are introduced to an organism. In embodiments, the quantitated, characterized, or separated cells are used in human disease treatments. In embodiments, the quantitated, characterized, or separated cells are further engineered, phenotyped, cultured or simultaneously tagged again.

In embodiments, the population of cells is a population of prokaryotic cells. In embodiments, the population of cells is a population of eukaryotic cells. In embodiments, the population of cells includes a population of adherent cells. In embodiments, the population of cells includes a population of non-adherent cells. In embodiments, the population of cells includes a population of adherent cells and a population of non-adherent cells. In embodiments, the population of cells is a population of adherent cells. In embodiments, the population of cells is a population of non-adherent cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1. Exemplary Process Description

### Sample Prep

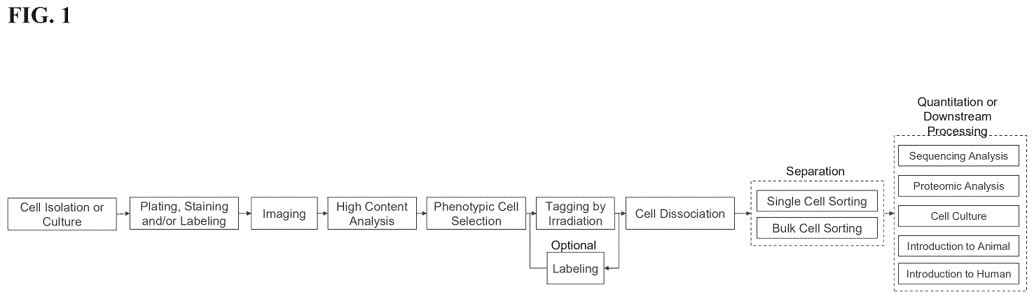

The workflow involved in tagging cells (FIG. 1) starts with a sample of cells that may have already been stained with fluorophores (e.g. organic dyes, antibody staining, ion-indicators, etc.), engineered to have fluorescent proteins, or have no prior staining. The details of this step are not illustrated in the figures. The pre-stained cells relate to or can indicate the presence of a certain phenotype. However, staining is not required to assess some phenotypes.

### Imaging

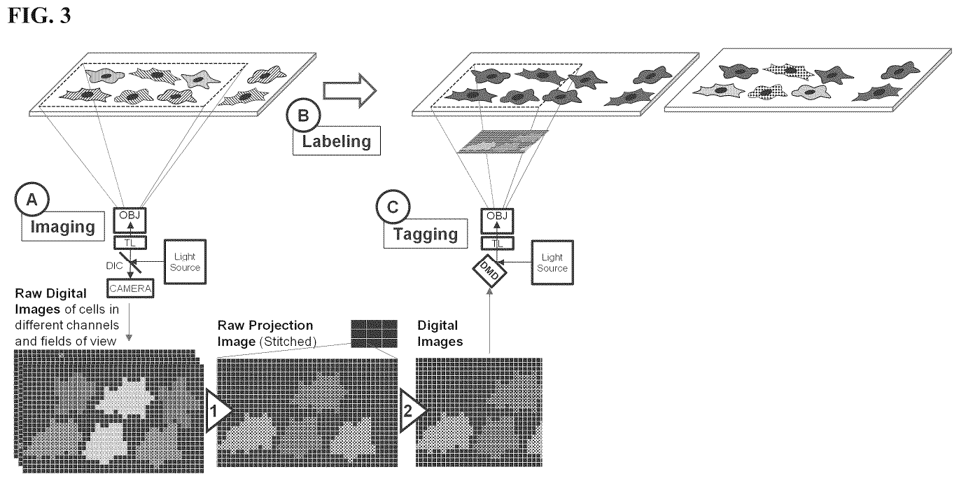

The sample is imaged on the microscope whereby raw digital images of various fields of view (FOVs) are collected in one or more color (Channels) by transmitted, fluorescence, or other microscopy imaging methods, where each image is a grayscale image with pixel values that represent the relative level of light interaction or fluorophore at a given spot on the sample (FIGS. 3 & 4, Step A). For example, in a fluorescence image, a bright pixel that lies inside or on a cell or sample implies a high level of the fluorophore in the location corresponding to the pixel within the cell or sample. For fluorescence imaging, oftentimes dichroic and filters are used to excite the sample at a given excitation wavelength and collect the emission light at a different (usually longer) wavelength. For transmitted imaging, the illumination light goes through the sample, and either grayscale images are collected or red, green, and blue images (each in grayscale) are collected and displayed together as an RGB (red, green, blue) ‘color’ image. The latter three images (RGB) can be collected at the same time on an RGB camera, as opposed to fluorescence imaging which uses only grayscale cameras.

### Labeling

For the purposes of this application (and unconventionally so), we define Labeling as the addition of the photosensitive entity that will later be used for Tagging. The Labeling step (FIGS. 3 & 4, Step B) may be performed before or after Imaging. Labeling as provided herein includes the addition of a chemical entity that binds directly or indirectly to the cells (or the biological entity), as well as indiscriminate illumination with certain wavelengths of light that activate the Label as needed in preparation for Tagging. Note that what distinguishes Labeling from Tagging is the indiscriminate nature of all that is done in Labeling as opposed to the discriminatory nature of all that is done in Tagging. For example, the light that may be used in Labeling to activate all photosensitive dyes irradiates all cells in the FOV without any selection or discrimination. Whereas, the light used for Tagging selectively irradiates cells of interest.

### Tagging

Tagging was done by the process of irradiating selected and non-selected cells in a discriminatory fashion to induce a photosensitive process at different levels in selected versus non-selected cells (FIGS. 3 & 4, Step C). In an example, the process's input were digital images that contained the intended irradiation doses for given micromirrors that corresponded to the selected or non-selected cells positions in the FOV (field of view).

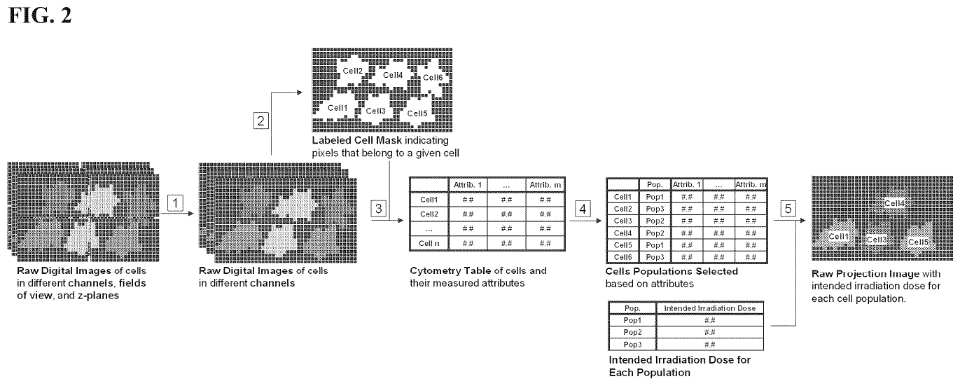

The first step of the process (FIG. 2 and FIGS. 3 & 4, arrowhead labeled 1) condensed the raw digital image information into a single Raw Projection Image where each pixel value represented the level of irradiation intended to be applied to the location of the sample corresponding to the position of the pixel in the FOV. Note that the dimensions of this image and the size of its pixels directly or indirectly corresponded to the pixels of the camera used to collect the images. Many variations of this process can exist, and most importantly include, Pixel-Based Processing and Cellular Analysis.

Pixel-Based Processing (FIG. 3, arrowhead labeled 1) is where digital values of pixels were subjected to mathematical operations without the identification of objects (cells) within the image. For example, a pixel of the Raw Projection Image was set to 0 if the corresponding pixel in the raw digital image, e.g. of a green fluorescence Channel, were below a preset threshold, and to 1, if above. The result was a binary Raw Projection Image with pixel values of 1 indicating a ‘high’ level of green fluorophore in this example. Using this Raw Projection Image resulted in tagging the areas (and effectively cells) that have high green. Note, no cells in the images were identified throughout this process. In other examples, pixel operations can be applied to images of two or more Channels, thereby combining the information content of multiple Channels to obtain Raw Projection Image.

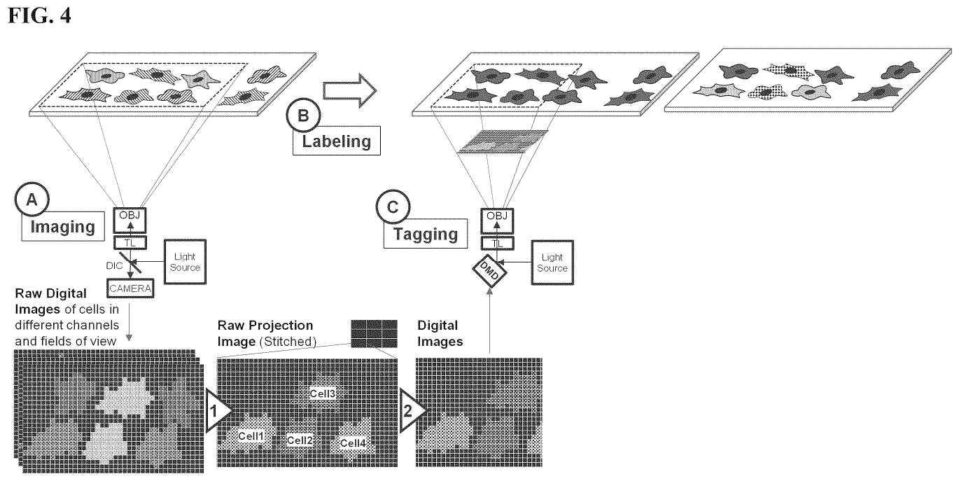

Cellular Analysis (FIG. 4, arrowhead labeled 1), represents another embodiment of the methods provided herein. For cellular analysis, the raw digital images were subjected to image analysis or computer vision processes to identify objects (cells) within them. A table of these cells (objects, hereinafter cells) was created in the process. Subsequently, quantifications based on pixel values and positions of each cell were made and added to the table as phenotypes of each cell. In an example, Phenotypes were defined based on morphometric and fluorometric features of the cells. Phenotypes can include without limitation, position (relative or absolute), texture, morphometric, fluorometric or complex metrics of each cell, as well as metrics based on their rate of change through time, and interactions of the cell with other cell(s) in the sample.

Based on criteria set by the user, this phenotype information was used to define one or more selected subpopulation of cells. These criteria can be set by software with limited to no input by a user. A Raw Projection Image was formed from the raw digital images of cells by only including the cells that belong to the particular selected subpopulation of cells and setting their pixel values to the irradiation dose intended for that selected subpopulation of cells. By repeating this for all selected subpopulations of cells, a Raw Projection Image was obtained with each of its pixel values representing the irradiation dose intended for each of a selected subpopulation of cells, if the position of the pixel corresponds to that of a pixel within that selected subpopulation of cells in the original raw digital image. Otherwise, the pixel value was set to a baseline value (e.g. 0 or max pixel value).

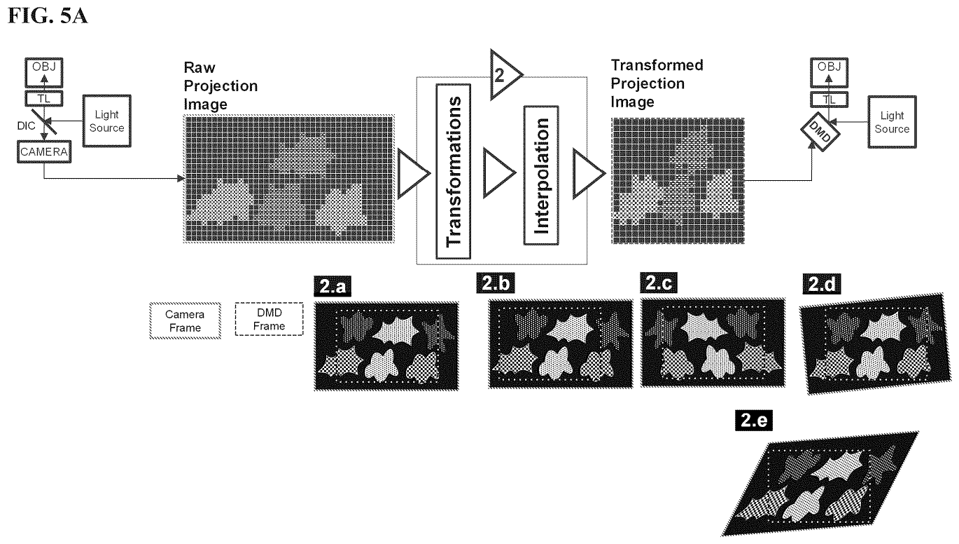

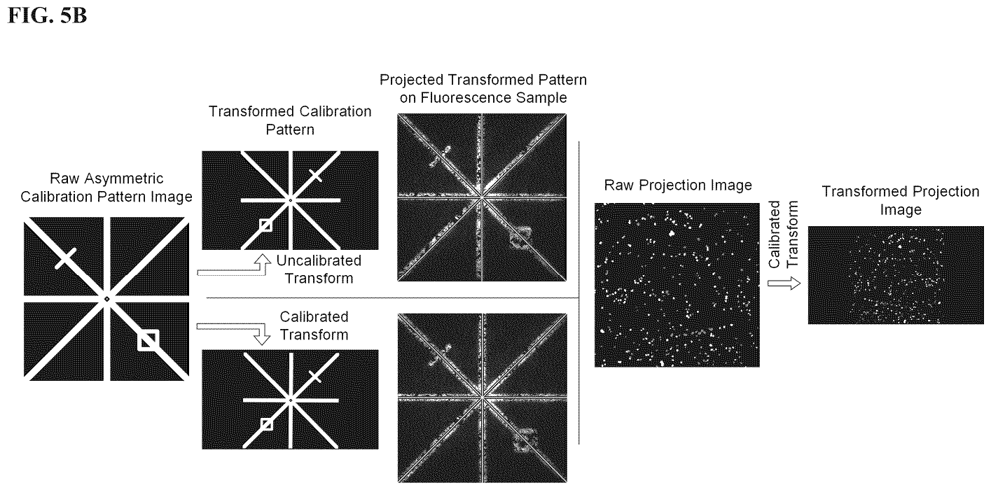

The second step of tagging (FIGS. 3 & 4, arrowhead labeled 2) took in Raw Projection Image and produced the Digital Image which may also be referred to herein as a mapped projection image. The terms “mapped projection image” and “digital image” as provided herein therefore, have the same technical meaning and may be used interchangeably throughout. The Raw Projection Image contains the information on the position of the pixels of the selected sub population of cells to irradiate and the light dose intended to be used to irradiate them. However, this information is in the context of the frame and pixel size of the camera chip (i.e. the dimension of Raw Projection Image is that of the camera chip dimensions or a derivation thereof). The information stored in the Digital Image is in the context of the frame and mirror size of the DMD chip. Naturally, a conversion between these two frames is needed and was applied. In embodiments, the light paths of the imaging camera and the DMD are not necessarily identical. Therefore, the optical aberrations inherent in the light paths may differ. The light paths may also have mechanical misalignments with respect to each other. The conversion between the two frames provided an opportunity to implement corrections for some of these inherent aberrations and misalignments. This conversion included a geometric transformation (FIG. 5) to reshape the image, followed by pixel interpolation to map each pixel to the DMD pixel coordinates and arrangement.

The transformation can take the form of cropping, Euclidean Transformations (translation, reflection, rotation, or others), affine transformations, projective transformations, or a combination thereof to correct for mechanical misalignments and optical aberrations (FIG. 5A). In examples herein, a combination of cropping, Euclidean Transformations (translation, reflection, and rotation), and affine transformations was applied to convert between the camera frame and the DMD chip, and to correct for optical and mechanical aberrations.

Pixel interpolation can follow transformation as, in some cases, oversampling of the input image is needed to allow the pixels to move to a position in between the allotted coordinates of the pixels in the original image during transformation. The pixel dimensions and arrangements of the Raw Projection Image and Digital Image may also be different, which provides another reason why the ability to calculate new values for pixels that fall in between original pixels is necessary when, for example, camera and DMD frames differ. In some examples, to calculate the value of new pixels that fall in between pixels in the original coordinates, a linear mathematical function (FIG. 6) was fitted to the neighboring pixel values. Other mathematical function of choice can be linear, cubic, or others in the case of one-dimensional fitting and bilinear, bicubic, or others in the case of two-dimensional fitting. The value of the function was calculated at the position of the intended pixel and used as the value for that pixel. In other examples, instead of fitting a mathematical function, the value of the nearest pixel in one dimension or two dimensions is simply used as the value for the new pixel.

### Example 2. Tables

TABLE 1
Depicts different embodiments of the steps of the methods provided herein.

| Transformation | Embodiments | Example Purpose |
|---|---|---|
| None | digital image = Raw Projection | pass through |
| | Image | |
| Cropping | digital image = a rectangular crop | To address the difference of frame |
| | of dimensions (W, H) at coordinate | and pixel sizes of the camera (raw |
| | (X, Y) from the raw image | digital image) and DMD (digital |
| Pixel | See Pixel Interpolation examples | image) |
| Interpolation | (FIG. 6). | |
| Geometric | Euclidean (Scaling, Translation, | To correct for mechanical mis- |
| Transformation | Reflection, and Rotation), Affine, | alignments, optical aberrations, |
| | and Projective Transformations | optical mis-matches, or other |
| | | sources of projection error between |
| | | camera, DMD, and sample. This |
| | | approach may require pixel |
| | | interpolation |
| Calibrated | Any transformation calculated | To ensure transformation corrects |
| Transformations | using a calibration target (e.g. | for mechanical mis- alignments, |
| | beads) by minimizing the | optical aberrations, optical mis- |
| | transformation needed to transform | matches, or other sources of |
| | a raw projection image to a digital | projection error in irradiation |
| | image or by projecting a calibration | system |
| | image and finding the | |
| | transformation that aligns the | |
| | projected image to the camera frame.. | |
| Translation | Digital image is a cropped subset of | To match the physical movement of |
| and/or Cropping | the larger raw projection image. In | the sample. To match physical |
| | examples, not all of raw projection | misalignments. |
| | image is calculated or accessed at | |
| | once. | |
| Obtaining pixel | Where pixel values of digital image | To enable image cytometry |
| locations and | are obtained from a data table of | |
| irradiation | identified cells in the raw image | |
| levels from a | and the pixels therein. | |
| lookup table | Where the digital image is a cell- | To calculate digital image based on |
| | mask that is calculated from image | complex cell selection criteria. |
| | cytometry table. | |

TABLE 2
Depicts different embodiments of irradiation exposure
useful for the methods provided herein.

| | Conducive to |
|---|---|
| Irradiation Unit | Simultaneous Tagging |
| Light Source + DMD | Yes |
| Light Source + Spatial Light Modulator | Yes |
| Light Source + Deformable Mirrors | Yes |
| Light Source + LCD Modules | Yes |
| Light Source + Galvanometer Scanner | No |
| Light Source + Acousto optic deflector | No |
| Light Source + Spinning Disk | No |
| Light Source + Multiphoton Microscope | No |

### Example 3. Exemplary Projection Image Mapping

The Raw Projection Image was transformed to correct for aberrations (such as chromatic, spherical and cylindrical) or motion, where the transformation can be calculated based on: 1) a calibration using a calibration sample of objects (e.g. beads), to assess mapping of pixel positions on the camera chip to mirror positions on the DMD. This is done for a single color (i.e. channel). A variation of the above calibration sample consists of multi-spectral objects (e.g. beads) to reduce the need for running many calibration routines. Or, In embodiments, iterative application of transformations to the projected target image of a known shape so that the shape's image onto the camera frame was orthonormal to the camera's pixels and frame dimensions (FIG. 5B) and 2) measurements or estimation of motion of sample to enable continuous tagging.

### Example 4. Exemplary Pixel Interpolation

After transformation, the pixels may not line up with the original coordinate system due to oversampling. Pixel interpolation was used to correct for this discrepancy. Pixel interpolation entailed fitting a mathematical equation, in examples herein a linear equation, to the existing points and using that equation to estimate the in-between value. In embodiments, the equation is the nearest neighbor formula (1D or 2D). In embodiments, the equation is otherwise linear or bilinear. In embodiments, the equation is cubic or bicubic.

### Example 5. Exemplary Continuous Tagging Processes

### Imaging Precedes Continuous Tagging

Digital images of various fields of view (FOVs) in one or more transmitted or fluorescence colors (Channels) are often acquired to identify areas that correspond to the Selected Subpopulation of Cells. In embodiments, those raw digital images are also necessary to identify the Selected Subpopulation of Cells. In this example the imaging step, therefore, precedes the tagging step. In embodiments, the imaging step immediately precedes the tagging step. In embodiments, the imaging of all or a large portion of the sample is completed in its entirety before tagging that portion is commenced (depicted below). In embodiments, the imaging of only a portion of the sample was completed before tagging that portion is commenced. In embodiments, the raw digital images were stored, and processed prior to the tagging.

### Continuous Tagging Process

Continuous tagging is the process of irradiating a selected subpopulation of cells in the sample, while the sample is physically in motion. This eliminated the need for the sample to accelerate/decelerate to move between fields of view, and increased throughput.

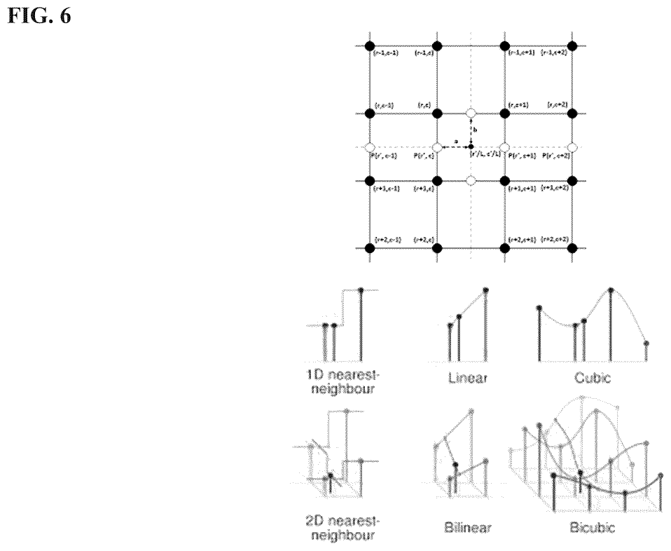

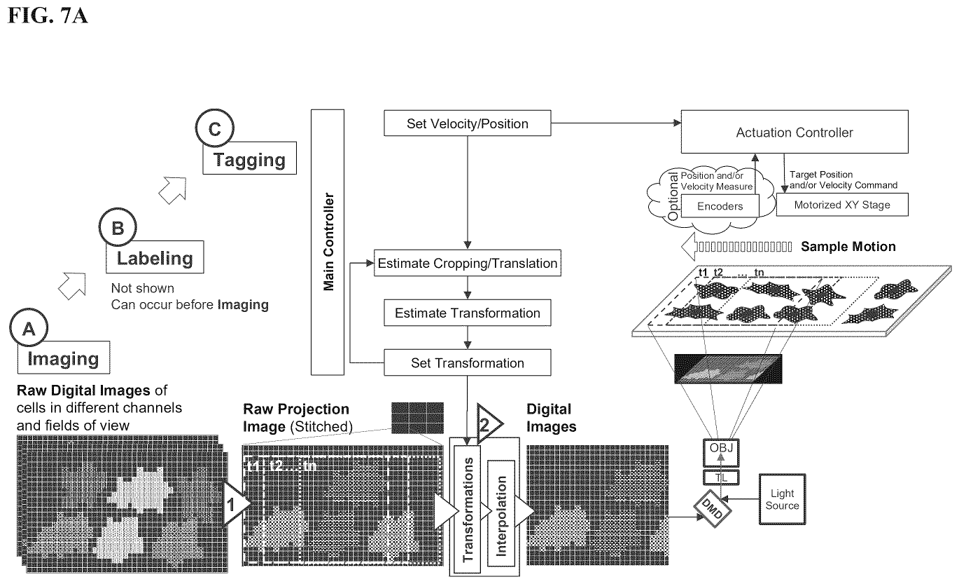

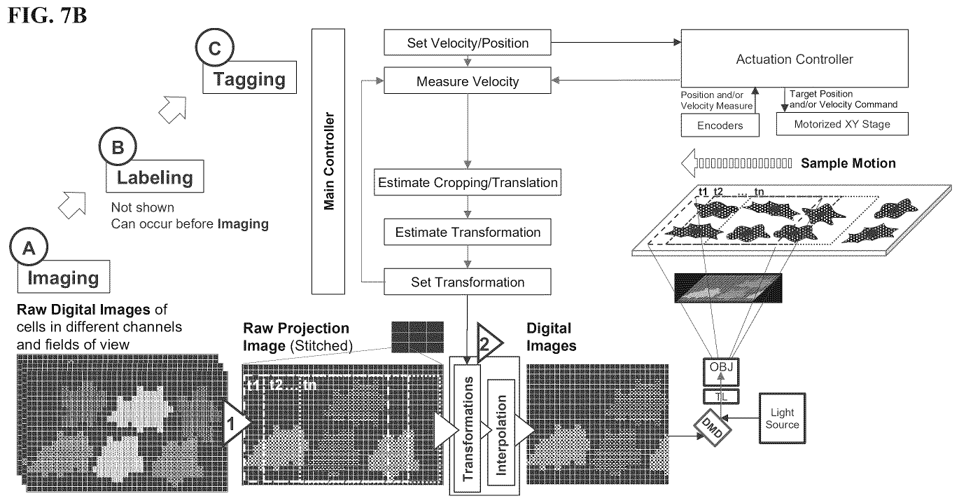

Principally, Continuous Tagging involved calculating the digital image from the Raw Projection Image in a manner and rate whereby the resultant digital image was synchronized with the movement of the sample. During irradiation, the sample was kept in focus using a surface-tracking autofocus system. In embodiments, other common methods of autofocus such as image-based, hardware, or software autofocus could be utilized. The Continuous Tagging irradiation method utilized a control loop between the movement of the sample and the generation and projection of the digital image, that was implemented as an open loop in some examples, or closed loop in others-respectively without or with repeated determination of the velocity of the sample. In either case, the transformation step as provided herein may be applied in each iteration of the loop, to create a digital image to attempt to match the movement of the sample. In embodiments, the transformation was applied a priori whereby, at each iteration, the loop selected an appropriate digital image from a plurality of transformed digital images to match the movement of the sample. This step took advantage of the cropping and/or translation options of the transformations as the movement of the sample corresponds to translation of the DMD frame across one or multiple raw digital images. In embodiments, the digital image contained the intended irradiation dose encoded as grayscale pixel values which were interpreted by the DMD and its controller as Duty Cycle of each mirror of the DMD, thereby delivering the intended dose of light to the intended selected population of cells during the time that that cell moves from one side of the field of view of the DMD to the other, defined as the maximum intended irradiation time (t_irrₘₐₓ). The stage velocity and the transformations (cropping and translation) were calculated respectively to achieve t_irrₘₐₓ and to match the projected frame with the position of the moving sample as closely as feasible. This was done by taking into account the maximum rate at which the DMD and its controller could project digital images (dmd_frame_rate), the pixel size of the camera as imaged on the sample (pixel_size) calculated as the dimension of a single physical pixel divided by the magnification of the imaging system, and the number of pixels in the axis of the camera frame corresponding to the axis of movement of the stage (camera_frame_size). The velocity of the stage (stage_velocity) was calculated as the product of camera_frame_size and pixel_size divided by t_irrₘₐₓ. The translation for each consecutive digital image (frame_period, or t1, t2, to tn in FIGS. 7A & 7B) was then determined to ensure a shift in pixels between each digital image calculated as camera_frame_size divided by the product of t_irrₘₐₓ and dmd_frame_rate. The transformations were calculated to also include a combination of cropping, Euclidean Transformations (translation, reflection, and rotation), and affine transformations to convert between the camera frame and the DMD chip and to correct for optical and mechanical aberrations, as well as the cropping and translation described above to account for the movement of the sample. In some embodiments, these transformations are applied as a single combined transformation. In embodiments, the transformations to correct for stage movement were applied prior to other transformations.

### Open-Loop Continuous Tagging

In the open-loop Continuous Tagging process (FIG. 7A), the sample (placed in a motorized microscope stage) is commanded to move at velocity determined as stage_velocity to ensure a maximum irradiation time of t_irrₘₐₓ (i.e. the time a given object spends traveling from one side of the FOV to the other). The effective cropping and/or translation needed to match this movement is calculated based on the predetermined value of stage_velocity and applied, in some examples with other transformations, to the Raw Projection Image to obtain digital images. Note that while the actual velocity of the sample at any given time may differ with this theoretical and commanded velocity due to mechanical and control imperfections and shortcomings, an encoded stage can be used to minimize this error. In embodiments, a non-encoded stage may be used with other means of ensuring constant velocity during the travel.

### Closed Loop Continuous Tagging

In the closed-loop Continuous Tagging process (FIG. 7B), the sample (placed in a motorized microscope stage) is commanded to move at a velocity determined as stage_velocity. The position and/or velocity of the stage at repeated intervals could then be reported via hardware or software communication to synchronize the projection of digital_images with stage movement. For example, in given time intervals, the position of the sample (or stage) was determined by an optical encoder, also possible using magnetic encoder or any sensor or accumulator of position. A signal was generated and shared with the DMD controller to synchronize the time at which the DMD would project the next digital image. Because stage_velocity was used to calculate the effective transformations needed to match the sample's movement, the tight control of the timing of the projection of each digital image ensured the intended dosage of light was commanded to the correct mirrors that corresponded to the selected subpopulations of cells at each time interval during the sample's movement across the field. The cropping and/or translation was applied, in some examples with other transformations, to the Raw Projection Image to obtain the digital image. In contrast to the open-loop process, this approach took into account variations in the velocity and incorporated them in calculating or selecting the digital image with the correct transformations for any particular time interval during stage movement. Therefore, the closed-loop process addressed inherent mismatch between the transformation applied and the actual position of the selected subpopulations of cells to be irradiated, resulting in higher accuracy of irradiation.

### Example 6. Simultaneous Tagging Processes

### Constant Intensity Simultaneous Tagging

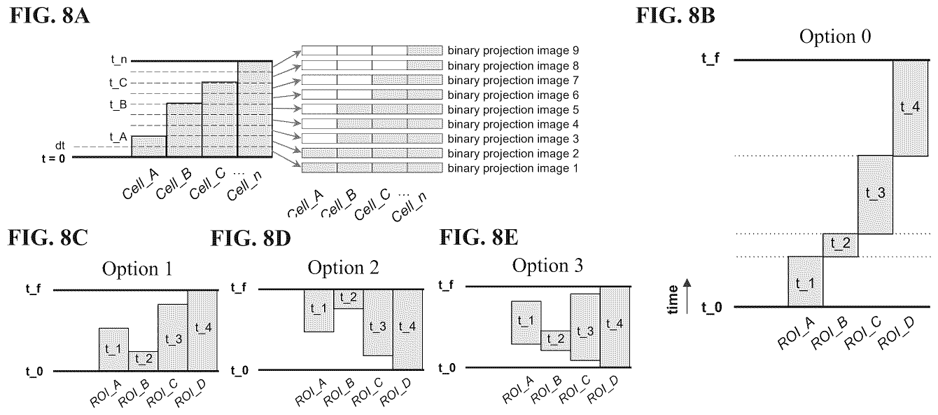

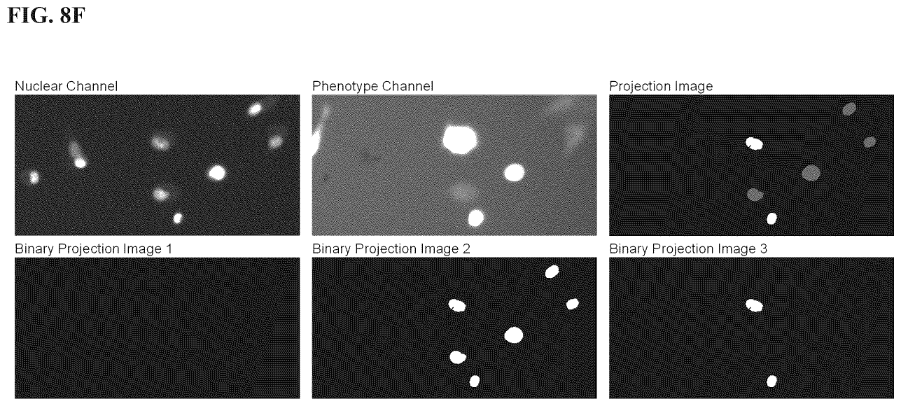

In some examples herein, the intended irradiation doses stored as pixel values in digital images were applied as irradiation times at a constant irradiation intensity, resulting in the intended irradiation dosage. The shortest time that a binary image can be completely projected using the DMD, dt, was then multiplied by the intended irradiation dose for each pixel (or cell) to give t_i, the intended irradiation time for that pixel (or cell), in some examples also multiplied or divided by a scaling factor, and in some examples also digitized to dt. The projection image was then processed as shown in FIG. 8A into a series of binary projection images such that each pixel of the binary projection image encoded whether that pixel (or cell) was to be irradiated at that time point, and the overlay of that pixel across all the binary projection images total in the intended irradiation time for that pixel (or cell). A single or a set of binary projection image was loaded into the memory of the DMD controller before each irradiation of the binary projection image or right before being needed per the irradiation time for each binary projection image. In some examples herein, when consecutive binary projection images were identical, instead of loading the same image repeatedly the irradiation time for that binary projection image was extended by a factor equivalent to the number of repeats to result in the same t_i. An Irradiation Event was the consecutive projection of all the binary projection images that make up a projection image. The Irradiation Event was initiated via software command in the examples herein, or could alternatively follow a hardware trigger in other embodiments. In embodiments, the start and stop of irradiation exposure of a given subpopulation is staggered relative to other subpopulations, while the irradiation intensity is maintained constant across subpopulations (FIG. 8B). In some examples herein, the start of irradiation exposure was initiated simultaneously for different subpopulations at the same irradiation intensity, but the stop time of irradiation exposure was changed across subpopulations (FIG. 8C). In embodiments, the start of the irradiation exposure varies across different subpopulations, while the irradiation intensity and stop time are constant across different subpopulations (FIG. 8D). In embodiments, the start and stop times of the irradiation exposure vary across different selected subpopulations of cells, but the irradiation intensity is constant for all subpopulations (FIG. 8E).

### Variable Intensity Simultaneous Tagging

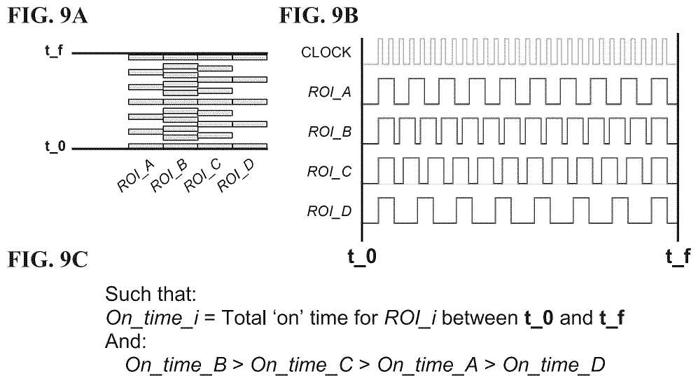

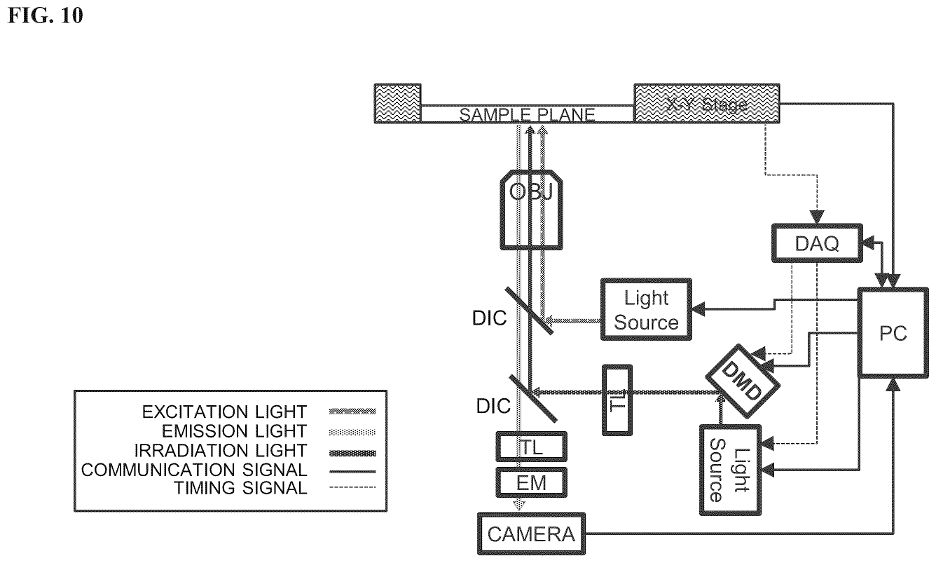

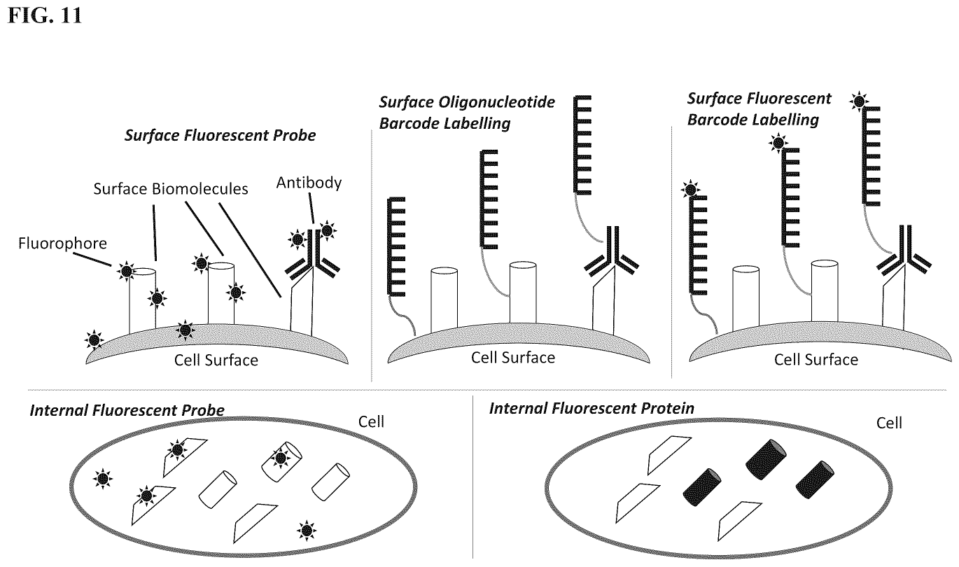

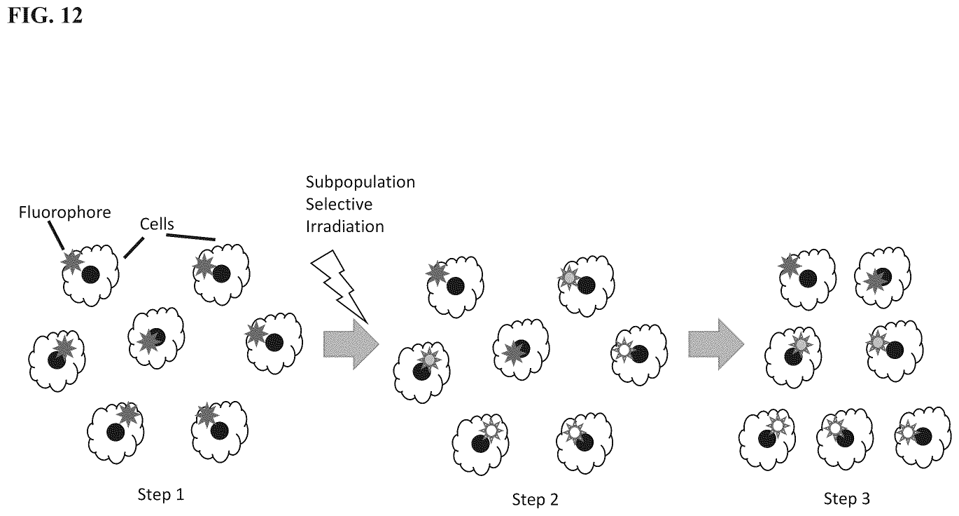

In embodiments, the intended irradiation doses stored as pixel values of digital image were applied as irradiation doses through Duty Cycle modulation. In embodiments, each pixel value in the digital image was directly proportional to the intended irradiation dose, as effected, for example, by a DMD micromirror on a corresponding position on the sample. Irradiation at the intended dose was accomplished by ensuring the corresponding micromirror on the DMD is set to the ON state for a length of time proportional to the intended irradiation dose. Irradiation at the intended dose was therefore accomplished by ensuring the duty-cycle of the corresponding micromirror was proportional to the intended irradiation dose, where the duty-cycle denotes the percentage of time that an individual micromirror is landed in the ON state versus the amount of time the same micromirror is landed in the OFF state. In examples herein, the DMD Controller computed and applied the Duty Cycle based on the grayscale pixel values of a given digital image. (FIG. 9) Individual or a set of projection images were pre-loaded into the memory of the DMD Controller before each Irradiation Event in discrete memory locations or in a shared memory buffer.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### Example 7. Three-Population Duty-Cycle Modulated Tagging Using Amphiphile Tags (Standard on Instrument Workflow)

Phenotypic Cell Staining: Cultured MDA-MB-231 cells (or other cell type, e.g., HeLa, MCF7, HEK293, or Mouse NSCs) were divided into three samples. The first sample was treated with a working solution of intracellular dye, for example, CellTracker™ Green (Thermo Fisher Scientific; prepared by diluting a 10 mM or 1-10 mM stock in DMSO in serum free medium or buffer to a 10 uM or 0.5-25 uM, followed by prewarming to 37° C.), the second sample was treated with a 20× dilution of the first sample staining solution, and the third sample was treated with the same working solution without the CellTracker™ product. The three cell samples were incubated 15-45 minutes under growth conditions. Cells were then washed with buffer (PBS, appropriate media, or appropriate serum free media to remove the CellTracker™ followed by incubation in media. The three populations were then treated with trypsin or accutase, diluted with media, mixed, and seeded into the wells of a 96 well microtiter plate (MTP) at a seeding density of 1,000-30,000 cells per well to create a single population of three stain phenotypes. The cells were then incubated in growth conditions for 1-3 days to form a monolayer or similar structure compatible with imaging.

Cell Labeling: Mixed cells were labelled with a red fluorescent, or caged red fluorescent, photoactive moiety. This label was introduced by tagging with a labelled antibody, a labelled membrane binding moiety (like a fatty acid, lipid, cholesterol, or PEG-lipid conjugate), or by direct introduction to the cell. For this example, the cells were labelled with a Photoactivatable Janelia Fluor 549 dye (PA JF549) conjugated to an amphiphile, DSPE-PEG100-Amine. The dye labelled amphiphile was synthesized by reacting NHS-PA JF549 (1.2-2 molar equivalents) with DSPE-PEG100-Amine (1 molar equivalent) in triethylamine (2 molar equivalents) in DMSO. The dye conjugated amphiphile was used without further purification. The photoactive amphiphile was kept as a stock in DMSO—and then diluted using buffer (such as PBS), media, or serum free media to 83 uM or a working concentration between 0.1 nM and 100 uM. The cells were then treated with the working concentration of the label for 30 min at room temperature or 37 deg C. The cells were then washed 1-3 times with media. Where noted, unlabeled, stained cells served as controls where no label was present in the buffer mixture, but analogous scaled solvents and buffers were used.

Experimental Conditions: The wells of the MTP are divided into three conditions, each with replicates:

TABLE 3

| | Condition A | Condition B | Condition C |
|---|---|---|---|
| Phenotypes | High Stain, Medium | High Stain Medium | High Stain Medium |
| | Stain, and Low Stain | Stain, and Low Stain | Stain, and Low Stain |
| Label | Control, No Label | With Label | With Label |
| Tagging: Cells | High Stain | None | High Stain |
| intended to be | | | |
| irradiated with | | | |
| a high dose | | | |
| Tagging: Cells | Medium Stain | None | Medium Stain |
| intended to be | | | |
| irradiated with | | | |
| a medium dose | | | |

### Cell Selection

Nuclear Staining: Optionally, to assist with cell segmentation, the cells were stained with a nuclear dye compatible with live cells as required. Hoechst (live), DAPI (fixed), DRAQ5 (live) or DRAQ7 (fixed) or other dyes compatible with downstream cell division were used. For this example, DRAQ5 was added in cell media at a final concentration of 5 uM or ranging from 0.1 to 20 M. Cells were then incubated for 5-60 minutes at room temperature or at 37 deg C., and then washed with buffer or media. The DRAQ5 can also be co-stained with the label in examples.

Image Acquisition: If growth medium of cells is not compatible with imaging (e.g. contains autofluorescent components), the medium was aspirated and replaced with PBS or other imaging media. The MTP with the cells seeded was then mounted on a fluorescence microscope and scanned. Cell images were captured in the channels corresponding to the stains, labels, tags and other supporting brightfield channels. Cell images were captured at all or a selected fields of all or a selected wells of a sample vessel such as a MTP where the image acquisition positions were calculated via a calibration lookup table produced by conventional means to ensure fields are tiled so that, after possible stitching processing, all areas of the selected area of the sample were covered by a field, such that no area was covered by more than one field. The navigation was aided by an encoded microscopy stage in this example or by other common means of controlled sample positioning in embodiments. Imaging was performed while the sample was kept in focus using a surface-tracking autofocus system (e.g. Nikon Perfect Focus System). In some embodiments, other common methods of autofocus such as image-based, hardware, or software autofocus could be utilized. In this example, fluorescence images were acquired in GREEN [Excitation: 475/28 nm, Emission: 515/30 nm], RED [Excitation: 542/33 nm Emission: 595/30 nm], and DEEP RED [Excitation: 631/28 nm Emission: 681/30 nm] channels using a 10× 0.45NA Nikon Plan Apo objective. In embodiments, air, oil, or water immersion objective with magnification of 0.5× to 100× and NA of 0.02 to 1.4 or similar objectives could be used. In embodiments, different objectives are used for image acquisitions and irradiation where the resolution transformation between raw projection images and digital images are applied along with other transformations as described in this example. Images were collected on a PCO Tech Panda 4.2 camera with a Scientific CMOS chip that had 2048×2048 pixels with a 6.5 um pixel pitch. Collected images were corrected for field-flatness by subtraction of an estimated background level and/or division by an estimated or calibrated shade image. The microscope was equipped with a light source capable of delivering light at about 500 mW from LEDs at given excitation and irradiation channels [390/22 nm, 475/28 nm, 542/33 nm, and 631/28 nm] through a liquid light guide and a condenser lens to illuminate a digital micromirror device (DMD) placed in an imaging conjugate plane. In embodiments, other light sources with a power output of 1 mW to 1000 mW are used with common modes of optically coupling the light source to illuminate a light patterning mechanism. In embodiments, other light patterning mechanisms are used. In embodiments, the total area of the light patterning mechanism as imaged onto the sample is selected to be smaller than, approximately equal to, or larger than the area of the camera chip as imaged onto the sample. In embodiments, the sampling rate of the light patterning mechanism (size of pixels or mirrors of the light patterning mechanism as imaged onto the sample) is selected to be smaller than, approximately equal to, or larger than the sampling rate of the camera (size of camera as imaged onto the sample). The DMD used had 1368×768 physical micromirrors with a 5.4 m micromirror pitch and was controlled by an evaluation board in conjunction with a PC, collectively defined as DMD Controller or DMD, and in some embodiments a component of Main Controller. The DMD was commanded to have all mirrors ON to approximately the same degree during Image Acquisition and to project the digital image while irradiating. In embodiments, the light path is changed such that light patterning mechanism is disengaged, removed or its patterning effect is otherwise diminished during image acquisition. The DMD Controller was capable of receiving software and hardware commands to control the timing of projections as well as communicating with the main controller for the purpose of transferring digital images.

### Phenotypic Cell Selection

Phenotypic Analysis: To characterize phenotype, as high, medium or low intensity GREEN stain, cell segmentation was performed by common means. For example, by applying a pixel value threshold (obtained from Otsu or similar methods) to obtain a binary mask followed by applying morphological filters to better align the boundary of the masks with the image of individual cells of nuclei. Individual cell or nuclear masks were then identified and labeled in software to represent individual cells or cell nuclei. Other common methods for cell and nuclear segmentation are available in commercial or open-source image analysis software packages (Cell Profiler, GE Harmony, Molecular Devices IN Carta and others). The Cell ID and the reference of pixels associated with that cell (either in pixel map, coordinates, or as reference to label mask values) were then collated for all the cells in a lookup table. The total pixel intensity of each identified cell was then quantified by summing the pixel value of the resultant image from the pixel-wise multiplication of the fluorescence image with the mask of the identified cell or nuclei. This total pixel value was divided by the value sum of the mask of the identified cell or nuclei (i.e. cell area or nuclear area) to produce the average pixel intensity of the identified cell in that fluorescence channel under its cell or nuclear mask. Cell area and/or nuclear area were also reported as a morphological feature. Phenotype of each cell was reported as the average or total pixel value of the GREEN fluorescence channel and added to the lookup table for each identified cell (FIG. 2 Steps 1-3).

Specific Cell Selection: A trinary phenotype for each cell was then defined by applying a user-selected threshold, Raw Phenotypic Threshold, on the total or average pixel intensity values of the stain channel (fluorometric feature) of the cells in the lookup table that had a cell or nuclear area (morphometric feature) within a user-selected range, thereby creating a selection of cells with a phenotype of high-GREEN (with total or average pixel intensity above a certain threshold), medium-GREEN (with a total or average pixel intensity between two thresholds) and low-GREEN (with total or average pixel intensity below a certain threshold) all of which had a cell or nuclear area within a range to select for healthy cells that are not seemingly engaged in cell death processes. An intended irradiation dose was determined for each of the subpopulations (Table 3) to enable sufficient distinction of tags in irradiated populations (FIG. 2 Step 4).

Creation of Raw Projection Image: Raw Projection Images were then generated by creating null images with the pixel dimensions of the camera, and assigning the intended irradiation dose, directly or as a proportional value, as pixel values to pixel coordinates of the pixels of each of the selection subpopulation of cells (FIG. 2 Step 5). The intended irradiation dose for each selected cell population first selected based on Table 3 for this example (and as defined in Table 5, 7, 9, 11, 13, 15, 17, 19, 21, 23, or 24 respective to each example provided) and in each example further adjusted to enable best separation of tagging in downstream processes. In this example, the relative intended irradiation dose of the ‘high dose’ was set to 1.0. The relative intended irradiation dose of the ‘medium dose,’ when present, was determined as 0.3 by iteratively irradiating labelled cells and observing the fluorescence of the tag to find the time at which the tag fluorescence achieved approximately 50% of the maximum fluorescence achievable in experimentally feasible time. The relative irradiation dose for the ‘low dose’ was set to 0.0. The cells that, as intended, received a high irradiation dose were tagged with a high tag, those that, as intended, received a medium irradiation dose received a medium tag, and those that, as intended, received a low irradiation dose received a low tag (FIG. 27). In other examples more than three subpopulations are distinctively tagged. Intended irradiation values for each pixel can be also adjusted according to an estimate or pre-calibrated measure of the shade (i.e. field non-uniformity) inherent in the irradiation light path to ensure all cells in a DMD field of view receive their intended irradiation level regardless of their position within the field. The pixel values of the raw projection image, in this example, were scaled such that the highest intended irradiation dose corresponded to 255 and the lowest to 0. This scaling can be specific to each of a light patterning mechanism in embodiments.

Transformation of pixel coordinates from cell images, or Raw Projection Images to Projection Images or vice-versa, included, for example, translation, cropping, affine, and geometric transformations. Whereas each pixel of the Raw Projection Image corresponded to a camera pixel, with this transformation, each pixel of a Projection Image or Digital Image corresponded to a micromirror in the DMD and each pixel value is set proportional or equal to the intended irradiation dose. This way, the corresponding cell population whose pixel coordinates or transformed pixel coordinates match the coordinate of the given pixel (micromirror) in the Projection Image received the intended dose that was encoded in the pixel value of the Projection Image. The translation component of the transformation could be obtained by calculating the difference between the recorded position where a particular cell image is acquired and the position where the projection image is, or to be, projected to irradiate cells. The cropping is calculated as the overlap of the projection of the camera chip and the DMD on the sample plane so that cropping removes areas of cell image that are not accessible by the DMD in any given field. A Calibrated Transformation that included at least cropping, translation and affine transformation was attained a priori by first projecting a calibration pattern image through the DMD onto a fluorescent target, in this case a very confluent monolayer of cells stained by a dye that excites with the line used to irradiate for tagging. Fluorescent images were obtained using the excitation wavelength used for irradiation to ensure the Calibration Transformation also corrected for the aberrations specific to the irradiation wavelength (FIG. 5 B). The calibration pattern included image features (i.e. an asymmetric shape) to make it easy to detect the location and orientation of the projected image of the pattern in the acquired image. A geometric transform was calculated to minimize the error (pixel-to-pixel coordinate difference of detected features) between the calibration pattern image and acquired calibration image. In examples herein, transformations were iteratively changed to provide an optimized transformation whereby the projected target image of the known pattern generated an image on the camera frame that was orthonormal to the camera's pixels and frame edges (i.e. aligned with the borders of the frame of the camera) (FIG. 5A). This transformation was recorded as the Calibrated Transformation and was used to convert between the camera frame and the DMD chip, to place the projection image in the correct position within the DMD frame, or vice versa in some embodiments, and to correct for optical and mechanical aberrations in the system.

### Cell Tagging

Irradiation Event: Each pixel value in the projection image were assigned directly proportional to the intended irradiation dose, as effected by a DMD micromirror on a corresponding position on the sample. Irradiation at the intended dose was accomplished by ensuring the corresponding micromirror on the DMD was set to the ON state for a length of time proportional to the intended irradiation dose. Irradiation at the intended dose was therefore accomplished by ensuring the duty-cycle of the corresponding micromirror was proportional to the intended irradiation dose, where the duty-cycle denotes the percentage of time that an individual micromirror is landed in the ON state versus the percentage of time the same micromirror is landed in the OFF state. In examples herein, the DMD Controller computed and applied the duty-cycle according to the grayscale pixel values of a given digital image and using its own internal clock for timing. The maximum irradiation time was enforced by the Main Controller that controlled the irradiation light source. Once individual or a set of projection images were transferred to the memory of the DMD Controller, and before turning ON the irradiation light (390/22 nm in this example or a wavelength within UV-VIS-NIR range of 190-3200 nm in embodiments), Irradiation Event was initiated via a software command issued by the Main Controller to the DMD Controller. This caused the DMD Controller to start projecting the projection image on the DMD chip and for the DMD pixels to assume the commanded duty-cycles. After a pre-set maximum irradiation time of 30 seconds in this example (0.0005-120 seconds in embodiments) had elapsed, the Main Controller turned OFF the irradiation light.

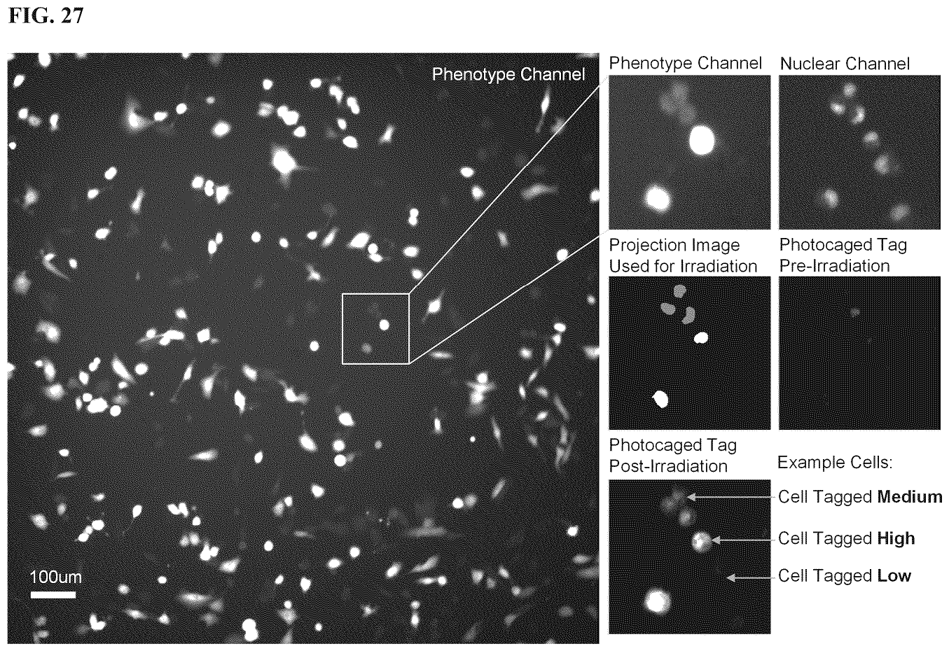

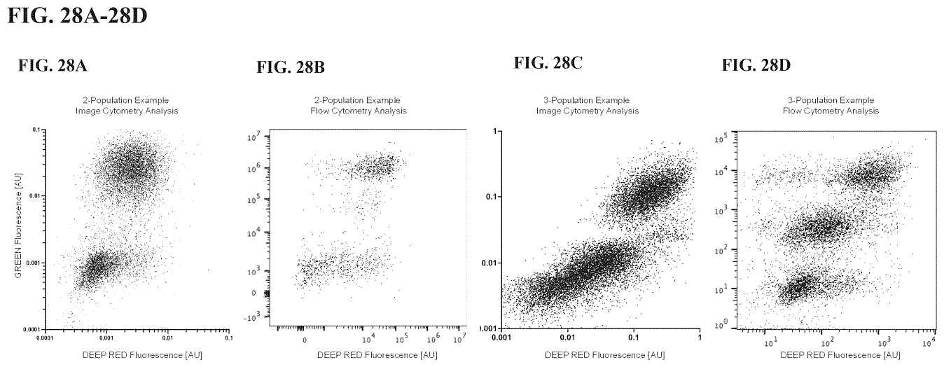

Irradiation: Following or as part of image acquisition, the MTP was moved by the stage to align the field of view to the intended irradiation position of each of the projection images. The navigation was aided by an encoded microscopy stage. The corresponding position of the projection image on the sample was calculated via a calibration lookup table which enabled automated imaging, processing and tagging of all or a selected fields of all or a selected wells of a sample vessel such as a MTP. This calibrated lookup table was produced by conventional means to ensure fields are tiled so that, after possible stitching processing, all areas of the selected area of the sample were covered by a field, such that no area was covered by more than one field. At each field position the corresponding projection image was transferred to the memory of the DMD controller and subsequently used to command each pixel of the DMD chip to assume a duty-cycle value, initiating an Irradiation Event. Once Irradiation Event and any subsequent imaging was completed for a given field, the stage was commanded to move to the next field in the look up table and the imaging and/or Irradiation continued for that field. This way, most or all cells in selected wells of a MTP were imaged and tagged without user interaction. Post-irradiation imaging and image cytometry was also performed either as a downstream process (FIG. 28C) or to assess the quality of Tagging prior to proceeding to other downstream processes (FIG. 28D) such as flow cytometry or FACS.

### Fluorescence Calibration Scan

Fluorescent standard beads with known fluorescence intensity can be used to convert the absolute fluorescence of each cell's tag to a relative fluorescence level that is co-relatable across different instruments.

Calibration Imaging: At least two but commonly three sets of fluorescently stained polystyrene or similar Calibration Beads (AccuCheck ERF Reference Particles) each with a well-characterized fluorescence emission level in the channel corresponding to the channel of interest are prepared such that each set has a specific fluorescence level different from the other set or sets. Separate sets of beads are prepared for each of the cell stain and label channels. For each channel, the beads are mixed and put down in at least one well of the MTP either by centrifugation of immobilization in a gel, before or after introducing the cells in to the MTP. The MTP is then mounted on a fluorescence microscope and fluorescence images of all wells with cells and the wells with beads are captured in the channel corresponding to the label (RED) and phenotypic cell stain (GREEN).

Calibration Bead Fluorescence Measurement: The fluorescence level of individual Calibration Beads are quantified by segmenting the acquired images and calculating the total pixel values in each segment, thereby creating a list of bead intensity values. Since the Calibration Beads come from a mixture of three standard bead sets, each with a known fluorescence level, three distinct populations are expected in the list of bead intensities. Simple thresholding, clustering, or Gaussian population unmixing methods are used to identify the three populations in the list. For each population, the mode is calculated as a Calibration Fluorescence Level.

Calibration Curve Fit: A linear, or when using more than three sets a nonlinear, function is fitted to the Calibration Fluorescence Levels as the independent variable and the expected standard fluorescence as the dependent variable. The coefficients of the function are saved as Source Calibration Coefficients for a given channel.

Tag Fluorescence Measurement: Each individual cell's coordinates and total tag fluorescence level were quantified by cell segmentation methods described above. Based on the coordinates, a matching cell with similar coordinates in the lookup table is identified. The total tag fluorescence level for the cell is added as a new column in the lookup table for that cell. The Tag Fluorescence Measurement can be used for calibration of Tag Thresholds. In some examples herein, the Tag Fluorescence Measurement before and after Irradiation was used to assess the quality of tagging.

Identification and Calibration of Tag Thresholds: Thresholds in the tag channel (RED) that best separate the three phenotypic populations low-GREEN, medium-GREEN and high-GREEN are identified as Raw Tag Threshold and inputted as independent variables into the function described by Source Calibration Coefficients to obtain Calibrated Tag Threshold values. In some examples herein, the Tag Thresholds were found as the threshold that best separated tagged populations on the flow cytometer.

Identification and Calibration of Phenotypic Thresholds: Threshold used in the cell selection process, Raw Phenotypic Threshold, is inputted as independent variable into the function described by Source Calibration Coefficients to obtain Calibrated Phenotype Threshold. In some examples herein, the Phenotypic Thresholds were found as the threshold that best separated the phenotypic populations on the flow cytometer.

### Cell Dissociation

The cells were then dissociated from the MTP for downstream processing. Enzymatic or enzyme-free methods were used to enable different downstream processes. For example, the cells were gently washed with PBS before adding 50 uL or 5-100 uL of Cell Dissociation Buffer (Thermo), trypsin, or accutase. Cells were bathed by rocking gently for 5 min or 1-15 min in room temperature or at 37 deg C. The cells and dissociation buffer were then resuspended in flow cytometry buffer or growth medium depending on the requirement of the next step. In another example, the cells were gently washed multiple times for 30 sec to 60 sec each with 5-50 uL of PBS before adding 5-100 uL of PBS and 0.5-10 mM EDTA. Cells were incubated for 1-10 min in room temperature or 37 deg C. The cells and dissociation buffer were then resuspended in flow cytometry buffer or growth medium depending on the requirement of the next step.

### Downstream Processing: Cell Separation

Calibration Bead Fluorescence Measurement: The previously described Calibration Beads can be run through the flow activated cell sorter (FACS) or flow cytometer and fluorescence levels are quantified without sorting. Similarly, positive and negative controls for the red tag and associated stains/labels used in the experiment were employed. Since the Calibration Beads come from a mixture of three standard bead sets, each with a known fluorescence level, three distinct populations are expected in the list of bead intensities. Simple thresholding, clustering, or Gaussian population unmixing methods are used to identify the three populations in the list. For each population, the mode is calculated as a Flow Calibration Fluorescence Level.

Calibration Curve Fit: A linear, or when using more than three sets a nonlinear, function is fitted to the Flow Calibration Fluorescence Levels as the dependent variable and the expected standard fluorescence as the independent variable. The coefficients of the function are saved as Flow Calibration Coefficients.

Calculation of Flow Gates: The previously saved Calibrated Tag Threshold values are used as independent variable inputs to the function described by the Flow Calibration Coefficients and the Calibrated Tag Flow Gate values are obtained. Similarly, calibrated Phenotype Threshold values are used as independent variable inputs to the function described by the Flow Calibration Coefficients and the Calibrated Phenotypic Flow Gate values are obtained. Positive and negative controls for the phenotype stains and tags used in the experiment can also be used and in the case of this experiment were employed.

Analysis and/or Sorting: The flow buffer was PBS or a buffer that includes one or more of 0.5% BSA, 5 mM EDTA, accutase, accumax, 12.5 mM HEPES, or other known flow and cell compatible buffer. Cells resuspended in the flow buffer were run through flow cytometry and sorting was done by using the Calibrated Tag Flow Gate values on the fluorescence channel corresponding to the tag fluorescence (RED, using a 554 nm laser), forming high-RED, medium-RED and low-RED populations. During sorting the fluorescence level of each cell in the channel corresponding to the cell stain used for phenotypic assessment is also measured (GREEN using a 488 nm or similar wavelength laser).

### Data Analysis

Using single cell data from the lookup table created during the Phenotypic Cell Selection process, the number of cells with RED and GREEN fluorescence levels within the three levels of the raw fluorescence thresholds prior to any tagging are quantified and labeled per Table 3 above.

For each sorted population, the number of cells with RED (Tag, 561 nm excitation with 579/16 nm emission filter) and GREEN (Phenotype Stain, 488 nm excitation with 513/26 nm emission filter) fluorescence levels within the three flow gates were quantified and labeled per Table 4 below. Gates were held constant across conditions. Percentages in parentheses refer to a per-condition basis.

Test condition C showed enrichment for Low Stain Low Tag, Mid Stain Mid Tag, and High Stain Hight Tag populations (Chi-squared of 3572 and p-value<0.0001 for Condition C) where expected while no such enrichment was observed in control conditions A and B.

TABLE 4

| | Condition A | | Condition B | | Condition C | |
|---|---|---|---|---|---|---|
| Low Stain Low Tag | 240 | (21%) | 349 | (40%) | 1551 | (23%) |
| Low Stain Mid Tag | 0 | (<1%) | 0 | (<1%) | 445 | (7.0%) |
| Low Stain High Tag | 0 | (<1%) | 0 | (<1%) | 104 | (1.5%) |
| Mid Stain Low Tag | 507 | (44%) | 306 | (35%) | 1040 | (15%) |
| Mid Stain Mid Tag | 1 | (<1%) | 2 | (<1%) | 1404 | (21%) |
| Mid Stain High Tag | 0 | (<1%) | 0 | (<1%) | 285 | (4.2%) |
| High Stain Low Tag | 410 | (35%) | 225 | (25%) | 240 | (3.5%) |
| High Stain Mid Tag | 0 | (<1%) | 0 | (<1%) | 361 | (5.4%) |
| High Stain High Tag | 0 | (<1%) | 0 | (<1%) | 1409 | (21%) |

| | | | |
|---|---|---|---|
| Total Counts | 1157 | 880 | 6839 |

### Example 8

### Phenotype Identification and Cell Tagging Using Pixel-Based Processing and Amphiphile-Based Tagging

### Experimental Description

Experimental Conditions: The wells of the MTP are divided into two conditions, each with replicates:

TABLE 5

| | Condition A | Condition B |
|---|---|---|
| Phenotypes | High Stain and | High Stain and |
| | Low Stain | Low Stain |
| Label | With Label | With Label |
| Tagging: Pixels intended to | None | High Stain |
| be irradiated with a high dose | | |

For the purpose of this Example, if not explicitly noted, all areas were performed as described in Example 7. For the purpose of Example 8, nuclear staining may be omitted and the following areas deviate from the procedure in Example 7.

### Cell Selection

Phenotypic Cell Staining: For this example, two stain phenotypes of MDA-MB-231 cells were made. One correlating to a high-GREEN phenotype by staining cells with 10 uM or 0.1-25 uM solution of CellTracker Green in PBS or media and a low-GREEN phenotype prepared by omitting the stain and treating a sample with only the working buffer. The cells were dissociated and combined as described in Example 7.

Cell Labeling: Mixed cells were labelled with a DEEP RED fluorescent, or caged deep red fluorescent, photoactive moiety. For this example, the cells were labelled with a Photoactivatable Janelia Fluor 646 (PA JF646) conjugated to an amphiphile, DSPE-PEG100-Amine. The dye labelled amphiphile was synthesized by reacting NHS-PA JF646 (1.2-2.0 molar equivalents) with DSPE-PEG100-Amine (1 molar equivalent) in triethylamine (2 molar equivalents) in DMSO. The dye conjugated amphiphile was used without further purification. The photoactive amphiphile was kept as a 2.5 mM stock or a 0.1 mM-10 mM stock and then diluted using buffer (such as PBS), media, or serum free media to a working concentration of 83 uM, 5 uM or between 0.1 nM and 100 uM. The cells were then treated with the working concentration of the label for 30 min at room temperature or 37 deg C. The cells were then washed 1-3 times with media.

Creation of Raw Projection Image: Raw Projection Images were generated by creating null images with the pixel dimensions of the camera, and assigning the intended irradiation dose, as pixel values determined through Pixel-Based Processing and as follows. A phenotype for each cell was defined based on whether or not a cell image contained pixels with values in certain ranges defined by the user. These values may be from a combination of one or more fluorescence channels. The user defined a single threshold based on pixel measurements. This threshold was applied on all pixels of the cell images to obtain a binary image wherein value of 1 corresponded to pixels in the cell image with intensity higher than the threshold and value of 0 correspond to pixels in the cell image with intensity lower than the threshold. An intended irradiation dose was determined for each of 0 and 1 (0.0 and 1.0 respectively in this example) to enable sufficient distinction of tags in irradiated populations and was assigned to the corresponding pixel in Raw Projection Image. In no step in creating the Raw Projection Images was cell or nuclear segmentation applied to identify individual cells.

Results: The following results in Table 6 were obtained by flow cytometry using GREEN (488 nm excitation, 525/40 nm emission filter) as a phenotype stain and DEEP RED (638 nm excitation, 660/10 nm emission filter) as a tag for gating.

Test condition B showed enrichment for the Low Stain Low Tag and High Stain High Tag populations (Chi-squared of 274 and p-value of <0.0001 for Condition B) where expected while no such enrichment was observed in control condition A.

TABLE 6

| | |
|---|---|
| Condition A | Condition B |

| | | | |
|---|---|---|---|
| High Stain High Tag | 0 | (<1%) | 387 (30%) |
| High Stain Low Tag | 558 | (43%) | 182 (14%) |
| Low Stain High Tag | 0 | (<1%) | 164 (13%) |
| Low Stain Low Tag | 727 | (57%) | 570 (43%) |

| | | |
|---|---|---|
| Total Counts | 1285 | 1303 |

### Example 9

### Phenotype Identification and Cell Tagging Using Staggered Irradiation and Amphiphile-Based Tagging

### Experimental Description

Experimental Conditions: The wells of the MTP are divided into three conditions, each in triplicate:

TABLE 7

| | Condition A | Condition B | Condition C |
|---|---|---|---|
| Phenotypes | High Stain, Medium | High Stain Medium | High Stain Medium |
| | Stain, and Low Stain | Stain, and Low Stain | Stain, and Low Stain |
| Label | Control, No Label | With Label | With Label |
| Tagging: Cells | High Stain | None | High Stain |
| intended to be | | | |
| irradiated with | | | |
| a high dose | | | |
| Tagging: Cells | Medium Stain | None | Medium Stain |
| intended to be | | | |
| irradiated with | | | |
| a medium dose | | | |

For the purpose of this Example, if not explicitly noted, all areas may be performed as described in Example 7. The following areas deviate from the procedure in Example 7.

### Cell Tagging

Creation of Raw Projection Image: In this example, the intended irradiation doses stored as pixel values of projection image were interpreted as irradiation times at a constant irradiation intensity. The shortest time that a binary image can be projected using the DMD, dt, was then multiplied by the indented irradiation dose for each pixel (or cell) to give t_i, the intended irradiation time for that pixel (or cell) digitized to the smallest time, dt, that the DMD could complete a projection event. The raw projection image calculated first based on the step outlined in Example 7, was then subsequently divided as shown in FIG. 8A into a series of binary projection images such that each pixel of the binary projection image encoded whether that pixel (or cell) was to be irradiated at that time point, and the overlay of that pixel across all the binary projection images total in the intended irradiation time, and therefore dose, for that pixel (or cell). A single or a set of binary projection image was calculated accordingly (FIG. 8F). An Irradiation Event was considered the consecutive projection of all the mapped binary projection images that make up a projection image. When consecutive binary projection images were identical, instead of loading the same image repeatedly, the irradiation time for that binary projection image was extended by a factor equivalent to the number of repeats to result in the same t_i. Binary projection images in which all pixels had the same value were not projected in some examples.

Irradiation Event: This step was performed as described in Example 7 with the exception of the following. Each pixel value in the projection images or mapped binary projection image corresponded to the intended irradiation state (ON/OFF) at any given time, as effected by a DMD micromirror on a corresponding position on the sample. Irradiation at the intended dose was accomplished by ensuring the corresponding micromirror on the DMD was set to the ON state for a length of time proportional to the intended irradiation dose. All the images in the set of projection images or mapped binary projection images were projected one at a time, each for the intended irradiation time t_i, a time proportional to the intended irradiation dose for that selected cell population, such that a relative intended irradiation dose of 0.0 corresponded to little to no irradiation and 1.0 corresponded to the maximum irradiation time. In this example, the relative intended irradiation time of the ‘high dose’ was set to 1.0. The relative intended irradiation time of the ‘medium dose’ was determined as 0.3 by following the same procedure as described in Example 7 to determine the relative irradiation dose of the ‘medium dose’ population thereof. The relative irradiation time for the ‘low dose’ was set to 0.0. The pre-set maximum irradiation time of 30 seconds in this example (0.0005-120 seconds in embodiments) was multiplied by each of these intended irradiation times to determine t_i for each selected subpopulation of cells. The Irradiation Event was initiated via software command and timed by the DMD Controller and the Main Controller. A single or a set of mapped binary projection image was loaded into the memory of the DMD controller before each irradiation or before being needed per the irradiation time for each mapped binary projection image. The start of irradiation exposure was initiated simultaneously for different subpopulations at the same irradiation intensity, but the stop time of irradiation exposure was changed across subpopulations (FIG. 8C). In other embodiments, the start and stop timing of each irradiation exposure can be set according to other variations in FIG. 8A-8E. At each t_i, if needed, the next individual or a set of projection images were transferred to the memory of the DMD Controller. The Main Controller issued a software command to DMD Controller to start projecting the next mapped binary projection image via the DMD chip and for the DMD pixels to assume the commanded duty-cycles. The timing of the projections and the maximum irradiation time was enforced by the Main Controller that controlled the irradiation light source. After a pre-set maximum irradiation time had elapsed, the Main Controller turned OFF the irradiation light.

Results. The following results were obtained as analyzed by flow cytometry and FACS using GREEN (phenotype stain) and RED (tag) gates.

Condition C showed enrichment for High Stain High Tag, Mid Stain Mid Tag, and Low Stain Low Tag populations (Chi-squared of 965 and p-value<0.0001 for Condition C) where expected while no such enrichment was observed in control conditions A and B.

TABLE 8

| | Condition A | | Condition B | | Condition C | |
|---|---|---|---|---|---|---|
| Low Stain Low Tag | 240 | (21%) | 349 | (40%) | 595 | (24%) |
| Low Stain Mid Tag | 0 | (<1%) | 0 | (<1%) | 166 | (6.7%) |
| Low Stain High Tag | 0 | (<1%) | 0 | (<1%) | 166 | (6.7%) |
| Mid Stain Low Tag | 507 | (44%) | 306 | (35%) | 129 | (5.2%) |
| Mid Stain Mid Tag | 1 | (<1%) | 2 | (<1%) | 410 | (17%) |
| Mid Stain High Tag | 0 | (<1%) | 0 | (<1%) | 341 | (14%) |
| High Stain Low Tag | 410 | (35%) | 225 | (26%) | 33 | (<1%) |
| High Stain Mid Tag | 0 | (<1%) | 0 | (<1%) | 166 | (6.7%) |
| High Stain High Tag | 0 | (<1%) | 0 | (<1%) | 473 | (19%) |

| | | | |
|---|---|---|---|
| Total Counts | 1157 | 880 | 2479 |

### Example 10

### Phenotype Identification and Cell Tagging of Two Cell Types and Amphiphile-Based Tagging

### Experimental Description

Experimental Conditions: The wells of the MTP were divided into two conditions, each in triplicate:

TABLE 9

| | Condition A | Condition B |
|---|---|---|
| Phenotypes | High Stain and | High Stain and |
| | Low Stain | Low Stain |
| Label | With Label | With Label |
| Tagging: Cells intended to be | None | High Stain |
| irradiated with high dose | | |

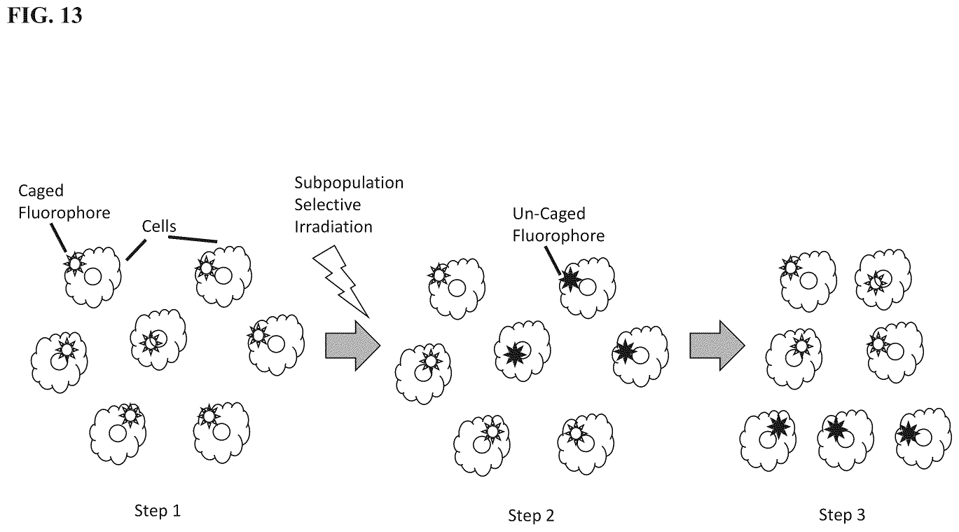

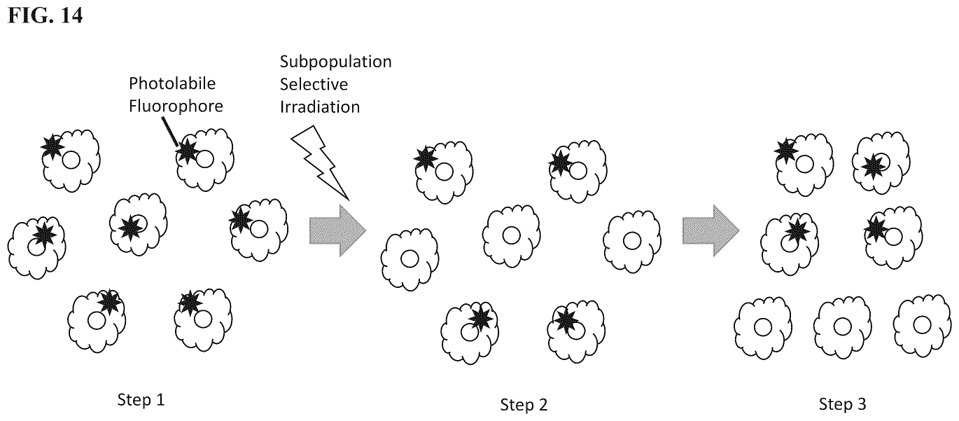

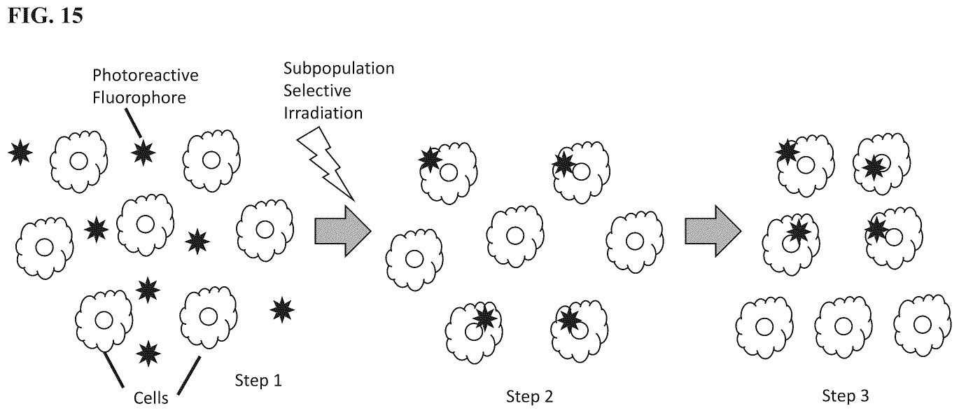

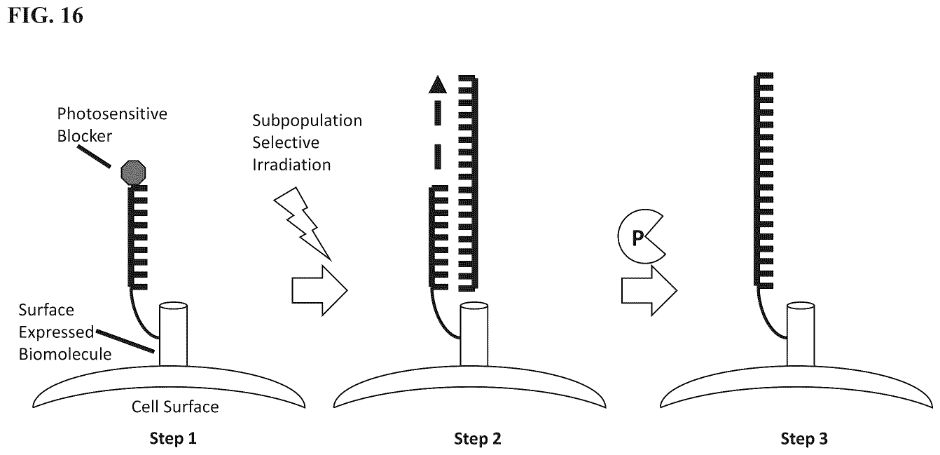

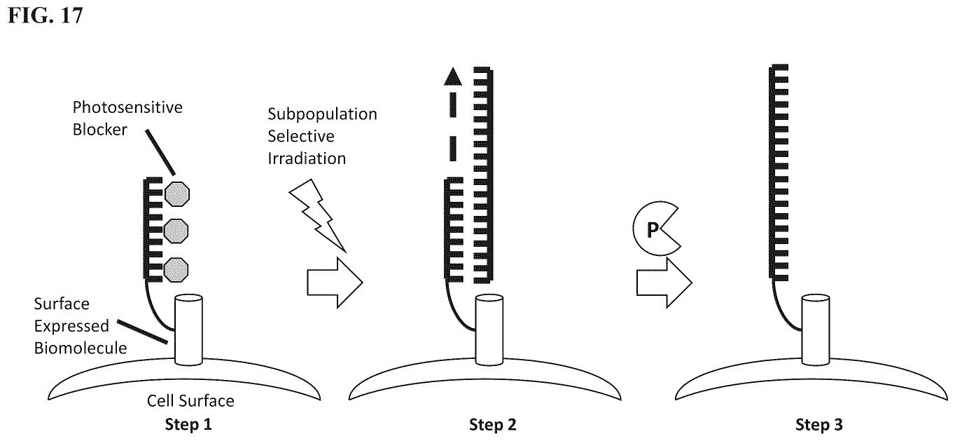

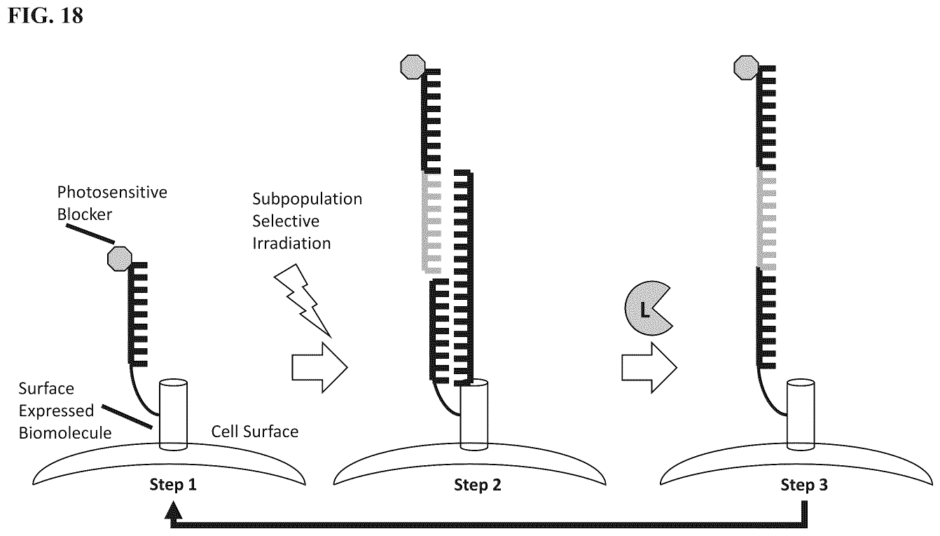

For the purpose of this Example, if not explicitly noted, all areas may be performed as described in Example 7. The following areas deviate from the procedure in Example 7. This workflow aligns with that depicted in FIG. 13.

Phenotypic Cell Staining: Cells of MBA-MD-231 and HEK-293T were cultured. The MDA-MB-231 was treated with a working solution of intracellular dye, for example, CellTracker™ Green (Thermo Fisher Scientific; prepared by diluting a 10 mM or 0.1-10 mM stock in DMSO in serum free media or buffer to 10 uM or 0.5-25 uM, followed by prewarming to 37° C.). The HEK-293T sample was treated with the same working solution without the CellTracker™ product. The two cell samples were incubated with the staining solutions for 30 min or 15-45 minutes under growth conditions. Cells were then washed with buffer (PBS), appropriate media, or appropriate serum free media to remove the CellTracker™ followed by incubation in media. The two cell types were then treated with trypsin or accutase, diluted with media, mixed, and seeded into the wells of a 96-well MTP at a seeding density of 1,000-30,000 cells per well to create a single population of three phenotypes. The cells were then incubated in growth conditions for 1-3 days to form a monolayer or similar structure compatible with imaging.

Cell Labeling: Mixed cells were labelled with a DEEP RED photoactive moiety. For this example, the cells were labelled with a Photoactivatable Janelia Fluor 646 (PA JF646) conjugated to an amphiphile, DSPE-PEG100-Amine. The dye labelled amphiphile was synthesized by reacting NHS-PA JF646 (1.2-2 molar equivalents) with DSPE-PEG100-Amine (1 molar equivalent) in triethylamine (2 molar equivalents) in DMSO. The dye conjugated amphiphile was used without further purification. The photoactive amphiphile was kept as a 2.5 mM stock in DMSO or a 0.1 mM-10 mM stock and then diluted using buffer (such as PBS), media, or serum free media to a working concentration of 83 uM, 5 uM or between 0.1 nM and 100 uM. The cells were then treated with the working concentration of the label for 30 min at room temperature or 37 deg C. The cells were then washed 1-3 times with media.

Phenotypic Cell Selection: All steps were performed as described in Example 7 except the medium-GREEN population was not included in the process.

Results: Both image cytometry (FIG. 28A) and flow cytometry (FIG. 28B) analyses were performed as downstream processes after tagging. The following results were obtained by flow cytometry using GREEN as a phenotype stain (488 nm excitation, 525/40 nm emission filter) and DEEP RED (638 nm emission, 660/10 nm emission filter) as a tag for gating.

Condition B showed enriched High Stain High Tag and Low Stain Low Tag populations (Chi-squared of 400 and p-value<0.0001 for Condition B) where expected while no such enrichment was observed in control condition A. This example illustrates the method, Cellular Analysis, whereby Tagging of a subpopulation of cells of one type selected solely based on phenotype results in the intended Tag being applied, approximately, only to those cells in the intended selected subpopulation and therefore type.

TABLE 10

| | Condition A | Condition B |
|---|---|---|
| High Stain High Tag | 5 (<1%) | 707(50%) |
| High Stain Low Tag | 1120 (47%) | 46 (3.2%) |
| Low Stain High Tag | 14 (<1%) | 306 (22%) |
| Low Stain Low Tag | 1234 (52%) | 362 (25%) |
| Total Counts | 2373 | 1421 |

### Example 11

### Phenotype Identification and Cell Tagging Using Closed-Loop Continuous Irradiation and Amphiphile-Based Tagging

### Experimental Description

Experimental Conditions: The wells of the MTP are divided into two conditions, each with replicates:

TABLE 11

| | Condition A | Condition B |
|---|---|---|
| Phenotypes | High Stain and | High Stain and |
| | Low Stain | Low Stain |
| Label | With Label | With Label |
| Tagging: Cells intended | None | High Stain |
| to be irradiated with a | | |
| high dose | | |

For the purpose of this Example, if not explicitly noted, all areas may be performed as described in Example 7. The following areas deviate from the procedure in Example 7.

Cell Tagging: In this example, Closed-Loop Continuous Tagging was used. Selected subpopulation of cells in the sample were irradiated while the sample was physically in motion. This eliminated the need for the sample to accelerate/decelerate to move between fields of view, and increased throughput. The position and/or velocity of the sample during movement was repeatedly communicated by the Actuation Controller (FIG. 7B) and used to synchronize Irradiation Events with stage movement. In some embodiments, an Open-Loop variation of this method can be implemented where the position and/or velocity of the sample during movement is estimated based on the commanded velocity (FIG. 7A) to synchronize Irradiation Events with stage movement.

Transformation of pixel coordinates from cell images, or Raw Projection Images to Projection Images were done as described in Example 7 except additional transformations were calculated and applied, as described in Continuous Tagging Process in Example 5, to account for the movement of stage, and therefore the relative translation of the DMD frame and the camera frame. A stage_velocity of 0.0222 mm per seconds was used in this example (or 0.001 to 60 mm per second in embodiments) for t_irrmax of 60 seconds in this example (or 0.0005-120 seconds in embodiments). A dmd_frame_rate of 2 frames per second was used (or 0.05-100000 frames per second in embodiments) to obtain a frame_period of 17 pixels (or 1-5000 pixels in embodiments). Devices with a DMD Controller capable of updating frames at higher rates could be used to irradiate samples at shorter t_irrmax and smaller frame_period. For example, using a DMD Controller with a dmd_frame_rate of 2048 frames per second can be used to irradiate at t_irrmax of 1 second and dmd_frame_rate of 1 pixel. In this example, translational and/or cropping transformations were applied to the raw projection images to obtain a series of projection images which represented a moving frame at equal intervals of 17 pixels to correspond with the stage movement during irradiation. Transformations, including translation or cropping, were used to calculate projection images that encompass portions of more than one original cell-image field of view.

Irradiation: Following image acquisition, a collection of projection images was calculated for a row or column of fields of view as described above. The MTP was then moved by the stage such that the objective's field of view aligned with a spot approximately one field of view center-to-center distance from the first field of view in a row or column of fields of view intended to be irradiated. The navigation was aided by an encoded microscopy stage. The corresponding position of the projection image on the sample could be calculated via a calibration lookup table. Upon a software command, the stage holding the MTP was commanded to move at the determined velocity of stage_velocity toward a spot overshooting approximately one field of view center-to-center distance from the last field of view in a row or column of fields of view intended to be irradiated. The microscope's surface tracking autofocus system was used to continuously keep the sample in focus. Other common methods of autofocus, such as image-based, software or hardware autofocus can be utilized. The initiation of the stage movement was synchronized with the initiation of the first Irradiation Event through software triggering with the appropriate and pre-calibrated timing delays. At this time, the Main Controller turned ON the irradiation light. Irradiation Events were initiated repeatedly, by a repeating hardware trigger. In this way, while the sample was moving, a series of Irradiation Events were initiated consecutively. At each Irradiation Event, the projection image is selected such that the estimated and transformed position of the center of the physical field of view at that time closely matches the center position of the selected projection image within the collection of projection images. This was done to minimize the positioning error between the irradiation pattern and the actual position of cells within the physical field of view.

Alternatively in an Open-Loop embodiment, where no hardware trigger produced by the Actuation Controller is used to select the next transformed projection image, a pre-calibrated or estimated time interval (frame_wait_period) is determined to correspond to the time the stage spends to travel a distance corresponding to frame_period multiplied by pixel_size. After initiation of the first Irradiation Event, each next projection image is then calculated or selected and loaded for projection at the given time intervals of frame_wait_period.

In the example herein, to produce the hardware trigger, the Actuation Controller was programmed to continuously monitor the position of the stage in the direction of movement using an optical encoder (50 nm resolution) and to alternate the state of a digital output at repeated position intervals corresponding to frame_period divided by pixel_size. This produced a hardware trigger at the intervals needed to initiate each Irradiation Event and change the frame projected by the DMD to match the speed of the stage as closely as feasible. At each Irradiation Event, a projection image was loaded onto the DMD chip. If appropriate, the light source can also be synched to ensure the DMD chip is not illuminated during the transition from one projection image to another. The projection image to be loaded in the next Irradiation Event was selected or calculated based on the predetermined or continuously-determined velocity or position of the stage or sample whereby this velocity or position is translated into the position of the projection image among the collection of projection images. In this example, upon receipt of each repeated trigger, the next projection image which was pre-calculated to be frame_period (e.g. 17 pixels) ahead was selected and loaded for projection. Offsets, delays, and correction coefficients, either calibrated or theoretically determined, were used in the calculation and operation of Irradiation Events to correct sources of mechanical and optical positioning errors. Once a row or column of fields were irradiated, and the stage arrived at its destination, the Main Controller turned OFF the irradiation light. The stage moved the sample to a position appropriate to start the next imaging and/or irradiation operation. In some examples, after irradiation, a set of images were acquired and image cytometry was performed as the downstream process or to assess the quality of irradiation before moving samples to downstream processes such as flow cytometry.

Results: The following results were obtained busing flow cytometry or FACS monitoring GREEN and RED gates as defined in Example 7.

Test condition B showed enriched Low Stain Low Tag and High Stain High Tag populations (Chi-squared of 79 and p-value<0.0001 for Condition B) where expected while no such enrichment was observed in control condition A.

TABLE 12

| | Condition A | Condition B |
|---|---|---|
| High Stain High Tag | 0 (0.0%) | 221 (34.0%) |
| High Stain Low Tag | 193 (34.4%) | 43 (6.6%) |
| Low Stain High Tag | 5 (0.9%) | 191 (29.4%) |
| Low Stain Low Tag | 363 (64.7%) | 195 (30.0%) |
| Total Counts | 561 | 650 |

### Example 12

### Phenotype Identification and Cell Barcoding Using Amphiphile-Linked Oligonucleotides

### Experimental Description

Experimental Conditions: The wells of the MTP are divided into five conditions, each in triplicate:

TABLE 13

| | Condition | Condition | Condition | Condition | Condition |
|---|---|---|---|---|---|
| | A | B | C | D | E |
| Phenotypes | High Stain and | High Stain and | High Stain and | High Stain and | High Stain and |
| | Low Stain | Low Stain | Low Stain | Low Stain | Low Stain |
| Label | Control, No | Control, No | Oligo Anchor | Oligo Anchor | Oligo Anchor |
| | Anchor Label | Anchor Label | Label | Label | Label |
| Tagging: Cells intended | None | None | None | High Stain | Low Stain |
| to be irradiated and | | | | | |
| tagged with Barcode 1 | | | | | |
| Tagging: Cells intended | None | None | None | Low Stain | High Stain |
| to be irradiated and | | | | | |
| tagged with Barcode 2 | | | | | |

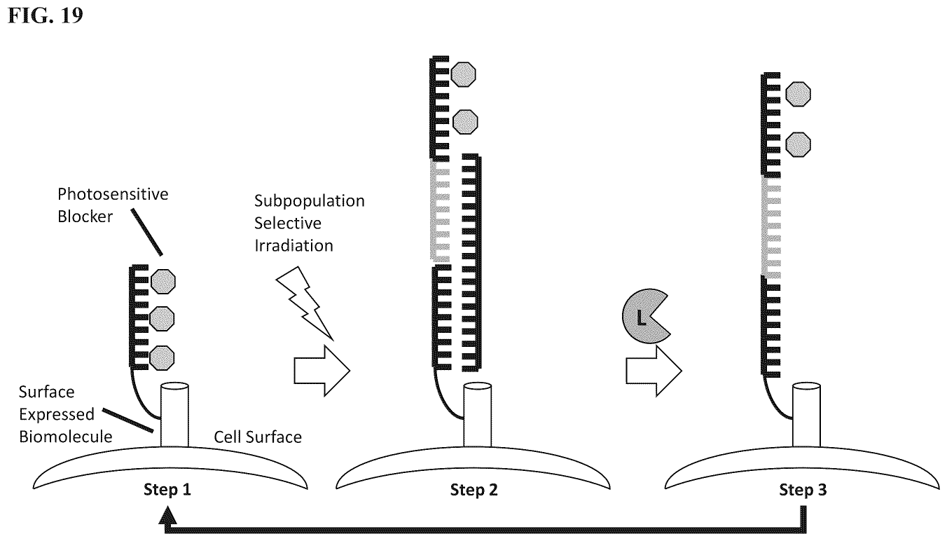

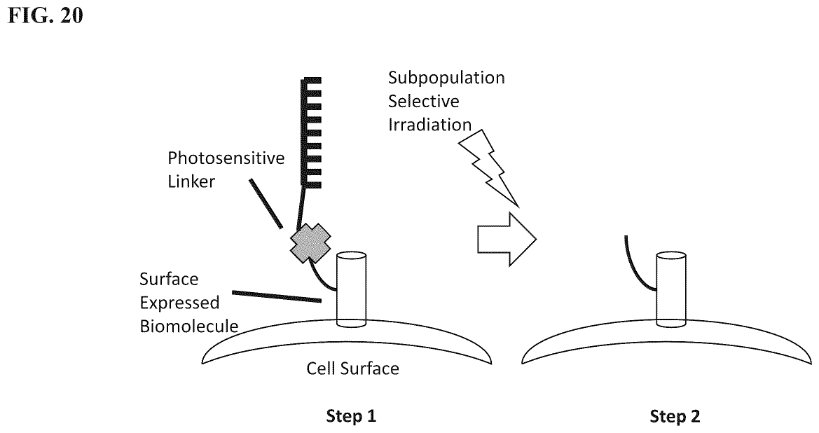

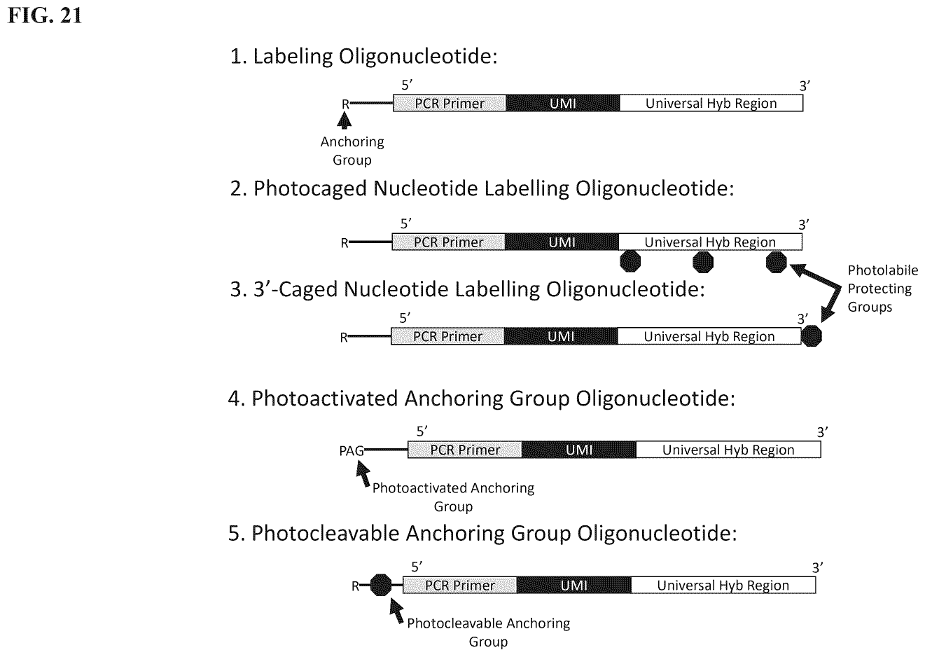

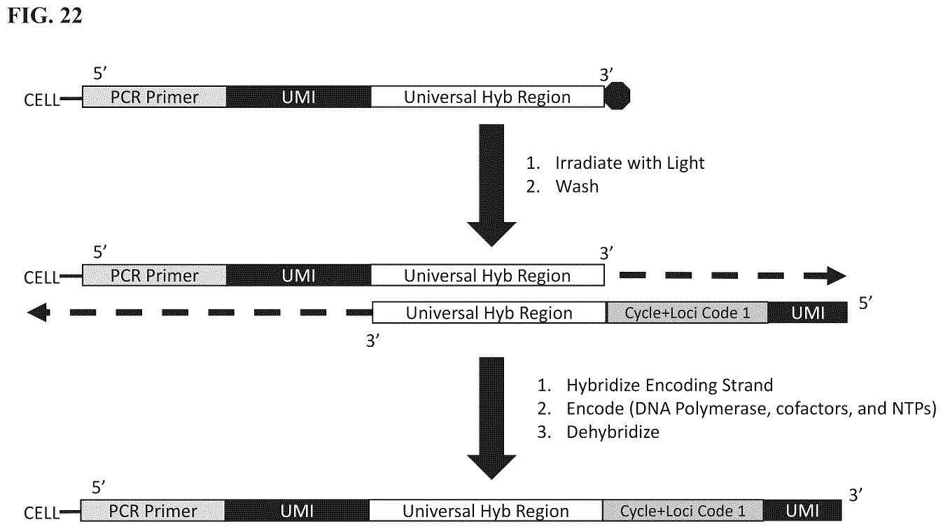

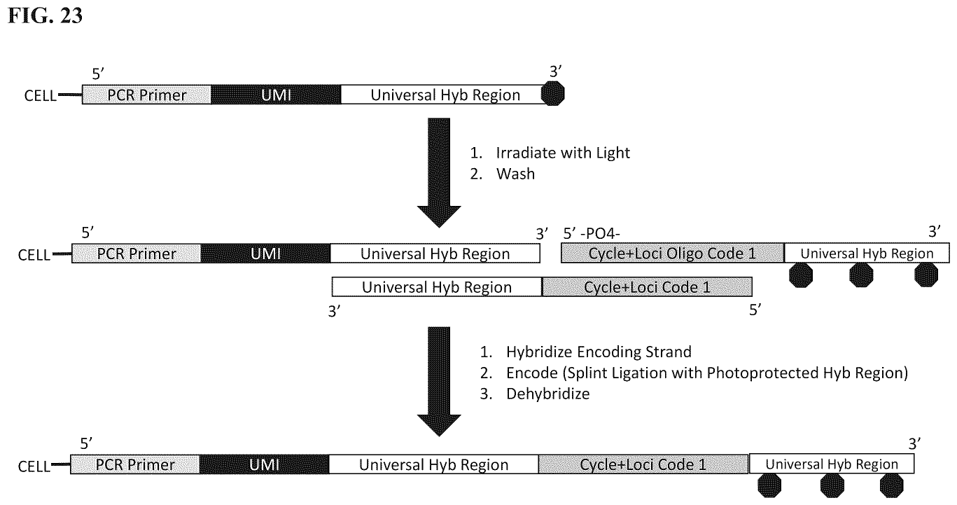

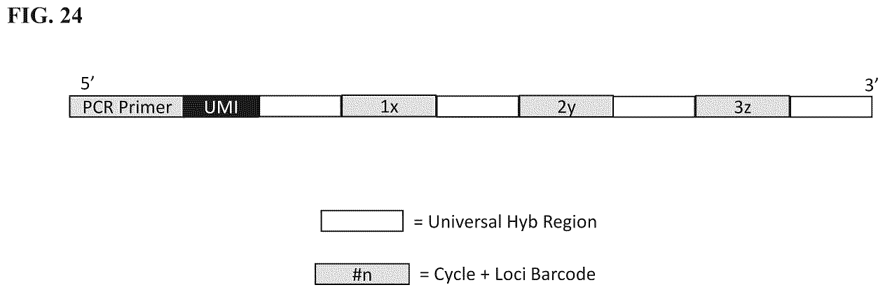

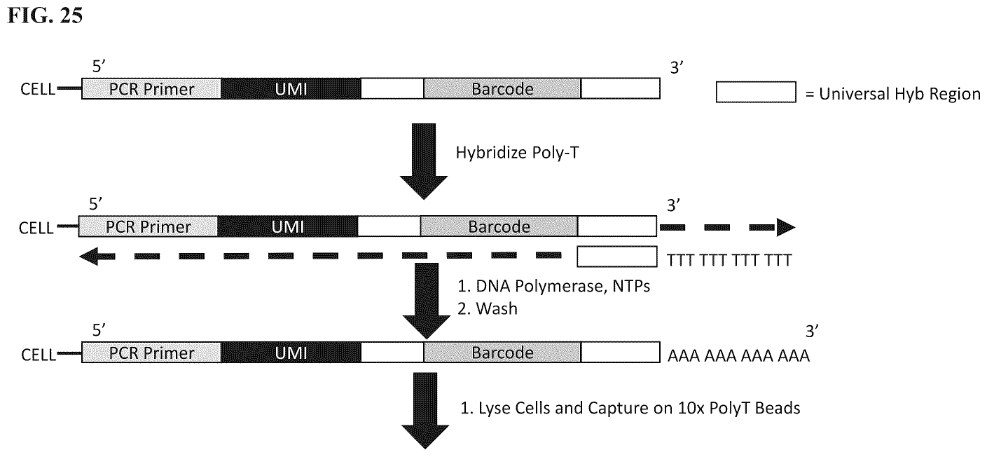

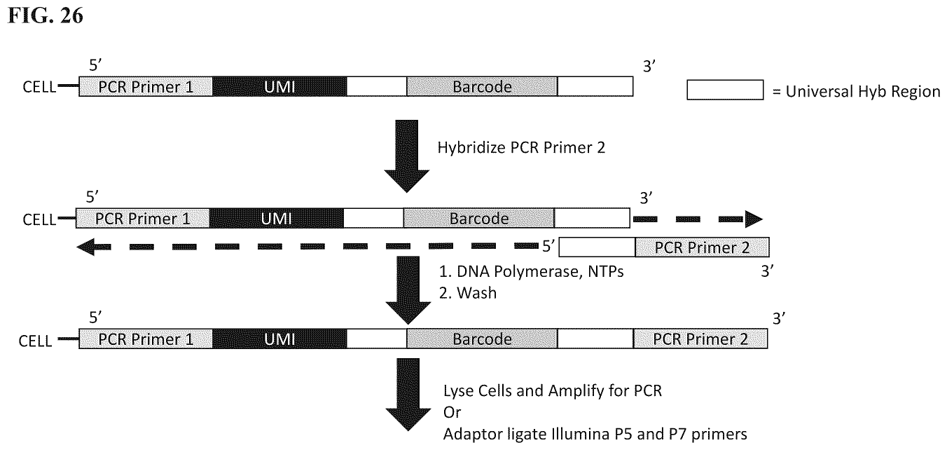

For the purpose of this Example, if not explicitly noted, all areas may be performed as described in Example 7. The following areas deviate from the procedure in Example 7. See FIG. 19 for a schematic of cellular barcoding by ligation step.

Cell Labeling: The wells of the MTP are divided into five conditions, each in triplicate: The stained cells to be labelled with a photoblocked oligonucleotide anchor (Conditions C, D and E) are treated with the oligonucleotide amphiphile DSPE-PEG100-Olignucleotide. The oligonucleotide can contain a PCR primer region and a universal anchor label sequence blocked with photolabile groups. The photocaged oligonucleotide is kept as a stock in DMSO (10 uM-10 mM) and then diluted using buffer, media, or serum free media to a working concentration between 0.1 nM and 100 uM. The cells are then treated with 20-200 uL of the diluted amphiphile oligonucleotide for 30 min at room temperature or 37 deg C. The cells are then washed 3 times with media. Unlabeled, stained cells (Conditions A and B) serve as controls where no amphiphile oligonucleotide is present in the buffer mixture, but analogous scaled solvents and buffers are used.

### Phenotypic Cell Selection and Cell Tagging

First Irradiation Event was performed as described in Example 7 except for the following. A pre-set maximum irradiation time of 90 seconds in this example (0.0005-120 seconds in embodiments) is used. Cells are washed and the “high GREEN phenotype” cells in Conditions A (control) and D are assigned to have a high intended irradiation dose with 390 nm wavelength light (or 405 nm or a wavelength within UV-VIS-NIR range of 190-3200 nm in embodiments) to selectively uncage the photolabile protecting groups from the oligonucleotide anchor labels.

First Barcoding Event All cells are then treated with a solution of 5′-phosphorylated DNA oligonucleotide (0.1-10 uM, Barcode 1+new photoprotected anchor label) and a DNA template (0.1-10 uM) in buffer (total volume 10-200 uL) that acts as a splint between the anchor label on the cell and the incoming Barcode 1 sequence. Each well is then treated with a mixture of T4 DNA ligase (NEB), T4 DNA Ligase Buffer (NEB) or other DNA ligase and remaining nuclease free water to a total 20-2500 uL volume. The cells are incubated at room temperature or 37 deg C. for 10 min to 2 hrs. The cells are washed of residual enzyme and oligos.

Second Irradiation Event: This step is performed as described in Example 7 except for the following. A pre-set maximum irradiation time of 90 seconds in this example (0.0005-120 seconds in embodiments) is used. The “low GREEN phenotype” cells in Conditions A (control) and D are assigned to have a high intended irradiation dose with 390 nm wavelength light (or 405 nm or a wavelength within UV-VIS-NIR range of 190-3200 nm in embodiments) to selectively uncage the photolabile protecting groups from those oligonucleotide anchor labels.

Second Barcoding Event: All cells are then treated with a solution of 5′-phosphorylated DNA oligonucleotide (0.1-10 uM, Barcode 2+new photoprotected anchor label) and a DNA template (0.1-10 uM) in buffer (total volume 10-200 uL) that acts as a splint between the anchor label on the cell and the incoming Barcode 2 sequence. Each well is then treated with a mixture of T4 DNA ligase (NEB), T4 DNA Ligase Buffer (NEB) or another DNA ligase and remaining nuclease free water to a total 20-250 uL volume. The cells are incubated at room temperature or 37 deg C. for 10 min to 2 hrs. The cells are washed of residual enzyme and oligos.

Third Irradiation Event: This step is performed as described in Example 7 except for the following. A pre-set maximum irradiation time of 90 seconds in this example (0.0005-120 seconds in embodiments) is used. In cell conditions B (control) and E, the “low GREEN phenotype” are assigned to have a high intended irradiation dose with 390 nm wavelength light (or 405 nm or a wavelength within UV-VIS-NIR range of 190-3200 nm in embodiments), uncaging the anchor label oligos in those samples.

Third Barcoding Event: All cells are then treated 5′-phosphorylated DNA oligonucleotide (0.1-10 uM, Barcode 1+new photoprotected anchor label) and a DNA template (0.1-10 uM) in buffer (total volume 10-200 uL) that acts as a splint between the cell anchor label oligonucleotide and Barcode 1. Each well is then treated with a mixture of T4 DNA ligase (NEB), T4 DNA Ligase Buffer (NEB) or another ligase and remaining nuclease free water to total 20-250 uL volume. The cells are incubated at room temperature or 37 deg C. for 10 min to 2 hrs. The cells are washed of residual enzyme and oligos.

Fourth Irradiation Event: This step is performed as described in Example 7 except for the following. A pre-set maximum irradiation time of 90 seconds in this example (0.0005-120 seconds in embodiments) is used. The “high GREEN phenotype” cells in Conditions B (control) and E are assigned to have a high intended irradiation dose with 390 nm wavelength light (or 405 nm or a wavelength within UV-VIS-NIR range of 190-3200 nm in embodiments) to selectively uncage the photolabile protecting groups from those oligonucleotide anchor labels.

Fourth Barcoding Event: All cells are then treated with a solution of 5′-phosphorylated DNA oligonucleotide (0.1-10 uM, Barcode 2+new photoprotected anchor label) and a DNA template (0.11-10 uM) in buffer (total volume 10-200 uL) that acts as a splint between the anchor label on the cell and the incoming Barcode 2 sequence. Each well is then treated with a mixture of T4 DNA ligase (NEB), T4 DNA Ligase Buffer (NEB) and remaining nuclease free water to a total 20-250 uL volume. The cells are incubated at room temperature or 37 deg C. for 10 min to 2 hrs. The cells are washed of residual enzyme and oligos.

Well-specific irradiation can occur simultaneously. That is the “high GREEN phenotype” in Conditions A and D can be irradiated and barcoded at the same time as the “low GREEN phenotype” in Conditions B and E.

In embodiments, all wells are then irradiated at 390 nm wavelength (or 405 nm or a wavelength within UV-VIS-NIR range of 190-3200 nm in embodiments) light to deprotect all remaining oligos prior to downstream processing.

Irradiation: The above Irradiation Events and Barcoding Events were repeated for all fields and wells of the sample. More iterations of the Irradiation Events and Barcoding Events can be done across and within samples to barcode cells.

### Data Analysis

The cells are subjected to a single polymerase extension event using the anchor label sequence as a complementary primer to create ssDNA complement strands of the barcodes attached to separated cells. The cells are then treated with base (50 uL, 0.1 M NaOH) to denature the duplex DNA and the supernatant containing ssDNA is collected, desalted and divided evenly between three sets PCR tubes. The first set of PCR tubes is treated with a Barcode 1 specific PCR primer (0.05-10 uM final concentration), the second set of PCR tubes is treated with a Barcode 2 specific PCR primer (0.05-10 uM final concentration), and the third set is treated with an anchor stem only primer (control, 0.05-10 uM final concentration). The solutions are then treated with Taq PCR reagents according to the NEB protocol, substituting buffer for the reverse primer. Following PCR cycling the resultant mixtures of DNA are subjected to standard gel electrophoresis to analyze for barcode incorporation.

Barcode analysis can also be performed by sequencing methods including single cell sequencing.

### Expected Results:

TABLE 14

| | | | | | Condition | | Condition | | Condition | |
|---|---|---|---|---|---|---|---|---|---|---|
| | | | | | C | | D | | E | |
| | Condition | | Condition | | Anchor | | Anchor | | Anchor | |
| | A | | B | | Label | | Label | | Label | |
| | No Anchor | | No Anchor | | No | | Test | | Test | |
| Note | Control | | Control | | Irradiation | | Condition | | Condition | |
| Stain | High | Low | High | Low | High | Low | High | Low | High | Low |
| BC1 | − | − | − | − | − | − | + | − | − | + |
| BC2 | − | − | − | − | − | − | − | + | + | − |
| Anchor | − | − | − | − | + | + | + | + | + | + |

### Example 13

### Phenotype Identification and Cell Barcoding Using Antibody-Nucleic Acid Conjugates

### Experimental Description

Experimental Conditions: The wells of the MTP were divided into four conditions, each with replicates:

TABLE 15

| | Condition A | Condition B | Condition C | Condition D |
|---|---|---|---|---|
| Phenotypes | High Stain | High Stain | High Stain | High Stain |
| | and Low | and Low | and Low | and Low |
| | Stain | Stain | Stain | Stain |
| Label | Negative | Positive | Control, No | Antibody |
| | Control, No | Control, | Irradiation | Treated, |
| | Splint | Unblocked | | Photocaged |
| | | Splint | | Splint |
| Tagging: Cells | High Stain | None | None | High Stain |
| intended to be | | | | |
| irradiated | | | | |

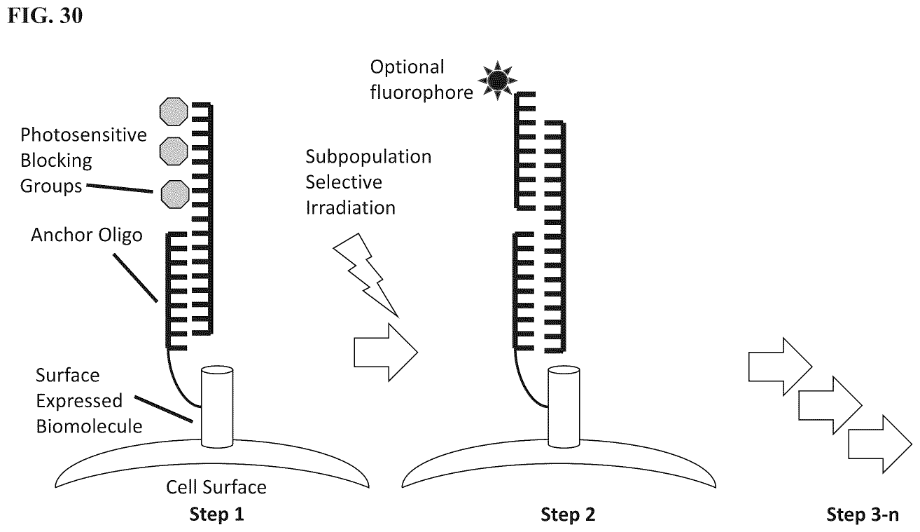

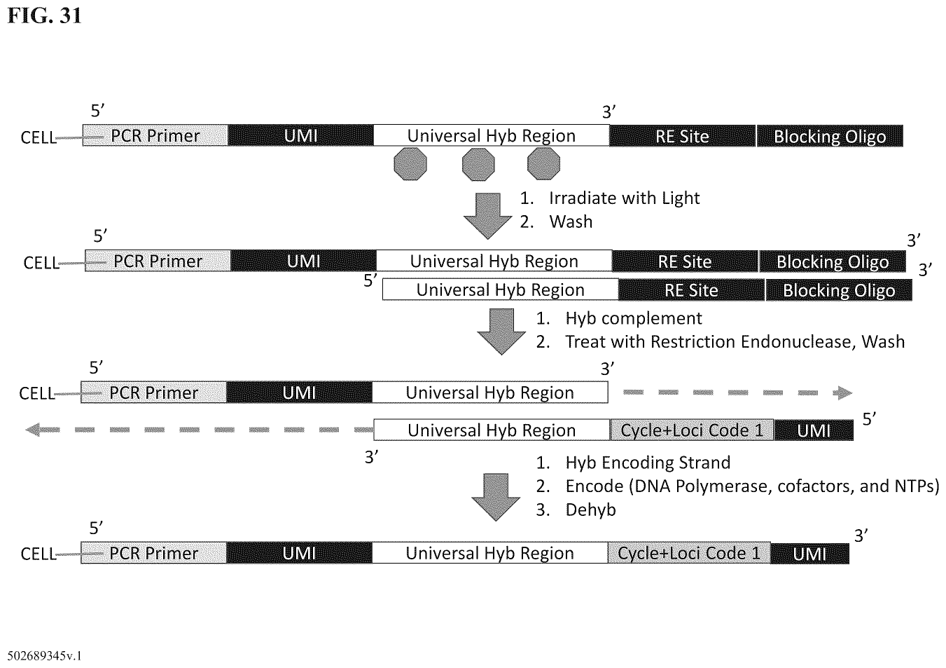

For the purpose of this Example, if not explicitly noted, all areas may be performed as described in Example 7. The following areas deviate from the procedure in Example 7. The following example is illustrated by the scheme in FIG. 30.

Phenotypic Cell Staining: Cultured MDA-MB-231 cells were divided into two samples. One sample was treated with a working solution of CellTracker™ Green (prepared by dissolving lyophilized CellTracker™ product in DMSO to a stock concentration of 10 mM and then diluting the resultant solution in serum free medium to 10 uM or between 0.5-25 uM, followed by prewarming to 37° C.). The second sample was treated with the same working solution without the CellTracker™ product. Both aliquots are incubated 30 min or 15-45 minutes under growth conditions. Cells were then washed to remove the CellTracker™ and dissociated (5 min, accutase at 37 deg C.). The two samples were then mixed, diluted with media and seeded into the wells of a 96 well MTP at a seeding density of 1,000-30,000 cells per well. The cells were then incubated in growth conditions for 1-3 days to form a monolayer or similar structure compatible with imaging.

Cell Labeling: Antibody-nucleic acid conjugates were prepared using the Thunder-Link Plus kit or Solulink kit to conjugate an 5′-amine terminated oligonucleotide containing DEEP RED fluorescent label for cell tracking to antiCD44 monoclonal antibody, or other relevant antibodies at a synthetic molar ratio or 1:10 or a range of 1:2 through 1:10 Ab:oligo. The 5′-amine terminated anchor oligonucleotide can contain a PCR region, an antibody specific barcode region, a dye for cell tracking, and an oligonucleotide anchor region that is protected with photolabile protecting groups. The conjugation procedure from the kit was followed, running for 2 h or up to overnight at room temperature and used without further purification. The antibody-nucleic acid conjugates were then diluted with buffer to a concentration of 0.5 mg/mL of Ab or 0.1-2 mg/mL of Ab stock for later use. For this example, the anchor oligonucleotide-Ab conjugate serves to anchor a photolabile protected splint oligonucleotide with a complementary region to the antibody-oligonucleotide. A photoblocked splint-oligonucleotide-Ab complex was formed by preincubating the photoprotected splint at a final concentration of 2 uM or 0.1-10 uM with the Ab-conjugate at a 1:20 dilution in media or a range of 1:10-1:200 dilution of a 0.1-2 mg/mL stock in buffer, media, or serum free media for 5 min or 5-60 min at room temperature or at 37 deg C.

The stained cells to be labelled with splint oligonucleotide-antibody conjugates (Conditions C and D) were treated with 50 uL or 20-200 uL of the antibody-nucleic acid per well for 30 min at room temperature. The cells were then washed with buffer or media and blocked with ssDNA (salmon sperm, 5 μg/mL working concentration in serum free media). Stained cells negative controls where no splint is present (Condition A) in the buffer mixture was performed by using analogous scaled solvents and buffers. Similarly, a positive control with a splint that lacked any photoreactive blocking groups was implemented for Condition B.

### Phenotypic Cell Selection and Cell Tagging

First Irradiation Event was performed as described in Example 7 except for the following. Following characterization of the cell phenotypes, the “high GREEN phenotype” cells in Conditions A and D were assigned to have a high intended irradiation dose with 390 nm wavelength light (or 405 nm or a wavelength within UV-VIS-NIR range of 190-3200 nm in embodiments).

First Barcoding Event: All cells were then treated with a solution of fluorescently labelled DNA oligonucleotide complementary to the previously photoblocked splint region 0.5 uM or a range of 0.1-10 uM (“Barcode 1”) in buffer, serum free media, or media. The fluorescent labelled DNA oligonucleotide can contain a PCR region, an antibody specific barcode region, a dye for cell tracking, an oligonucleotide anchor region that is protected with photolabile protecting groups, or an additional complementary region for an additional photoprotected splint to bind. For this example, the fluorescent labelled barcode was made up of a complementary region to the unblocked splint as well as a TAM fluorophore to serve as a tag (RED). The cells were incubated at room temperature or 37 deg C. for 10 min or up to 2 hrs. The cells were washed 1-3 times with buffer, media, or serum free media.

Irradiation: The above Irradiation Events and Barcoding Events were repeated for all fields and wells of the sample.

Data Analysis: The cells were treated with accutase (5 min, 37 deg C.) and diluted with media. Like samples were combined and the four conditions were subjected to flow cytometry or FACS monitoring the tag fluorescence (RED) and phenotype stain (GREEN).

Results: Test condition D showed enrichment in Low Stain Low Tag and High Stain High Tag (Chi-squared of 257 and p-value<0.0001 for Condition D) where expected while no such enrichment was observed in negative controls condition A and condition C. The High Stain High Tag population was similar to that of the positive control Condition B. However, condition D also showed a separate Low Stain Low Tag population.

TABLE 16

| | Condition A | Condition B | Condition C | Condition D |
|---|---|---|---|---|
| | No Splint | Unblocked | Anchor Label, | Anchor Label, |
| | | Splint | Blocked Splint | Blocked Splint |
| Note | Negative Control | Positive Control | No Irradiation | Test Condition |
| High Stain High Tag | 3 (<1%) | 420 (45%) | 3 (<1%) | 539 (18%) |
| High Stain Low Tag | 380(41%) | 4 (<1%) | 561 (49%) | 464 (15%) |
| Low Stain High Tag | 2 (<1%) | 496 (53%) | 17(1.5%) | 499 (16%) |
| Low Stain Low Tag | 543 (59%) | 17 (1.8%) | 568 (49%) | 1541 (51%) |
| Total Counts | 928 | 937 | 1149 | 3043 |

### Example 14

### Phenotype Identification and Cell Tagging Using Activation of Photocaged Fluorescently Labelled Antibodies

### Experimental Description

Experimental Conditions: The wells of the MTP were divided into two conditions with replicates:

TABLE 17

| | Condition A | Condition B |
|---|---|---|
| Phenotypes | High Stain and | High Stain and |
| | Low Stain | Low Stain |
| Label | With Label | With Label |
| Tagging: Cells intended to be | None | High Stain |
| irradiated with high dose | | |

For the purpose of this Example, if not explicitly noted, all areas may be performed as described in Example 7. The following areas deviate from the procedure in Example 7. This workflow is depicted in the scheme in FIG. 13.

Phenotypic Cell Staining: Cultured MDA-MB-231 cells were split into two samples. One sample was treated with a working solution of CellTracker™ Green (prepared by dissolving lyophilized CellTracker™ product in DMSO to a stock concentration of 10 mM or 0.1-10 mM and then diluting the resultant solution in serum free medium to 10 uM or 0.5-25 uM, followed by prewarming to 37° C.). The second sample was treated with the same working solution without the CellTracker™ product. Both aliquots were incubated for 30 min or 15-45 minutes under growth conditions. The two samples were then dissociated, mixed and seeded into the wells of a 96 well MTP at a seeding density of 1,000-30,000 cells per well. The cells were then incubated in growth conditions for 1-3 days to form a monolayer or similar structure compatible with imaging.

Cell Labeling: The stained cells to be labelled with a fluorescent label were incubated with a Photoactivated JF646 (PAJF646) tagged antiCD44 antibody (1:20 dilution or a 1:10-1:200 dilution of a 2 mg/mL or a 0.1 mg/mL-2 mg/mL stock solution in buffer). The tagged antibody was synthesized by reacting 12 or 1-20 molar equivalents of NHS-PAJF646 in sodium carbonate buffer with a 1 mg/mL concentration of antibody. The caged-fluorescent antibody was purified by commercially available gel spin column. After incubating the cells with the PAJF646-antibody for 30 min or 5-60 min at room temperature or 37 deg the cells were then washed 1-3 times with buffer or media.

### Cell Selection

Nuclear Staining: To assist with cell segmentation, the cells were stained with a RED nuclear dye compatible with live cells and cell division as required by downstream processes, SYTO Orange 82 (Thermo) at a concentration of 2 uM in media or 0.1-10 uM in buffer, media, or serum free media.

### Phenotypic Cell Selection and Cell Tagging

Specific Cell Selection: The cells are washed and in Conditions B, the “high GREEN phenotype” cells were assigned to have a high intended irradiation dose whereas the “low GREEN phenotype” cells were assigned to have a low intended irradiation dose or no dose at all. Condition A remained unirradiated.

### Downstream Processing

Analysis by Flow Cytometry: The flow cytometry buffer was PBS or a buffer that includes one or more of 0.5% BSA, 5 mM EDTA, 12.5 mM HEPES, or other flow compatible buffer. Cells resuspended in the flow cytometry buffer were run through flow cytometry values on the fluorescence channel corresponding to the tag fluorescence (DEEP RED, using a 633 nm or similar laser), forming high-DEEP RED and low-DEEP RED populations. During analysis the fluorescence level of each cell in the channel corresponding to the cell stain used for phenotypic assessment is also measured (GREEN using a 488 nm or similar laser).

Data Analysis: Using single cell data from the lookup table created during the Phenotypic Cell Selection process, the number of cells with DEEP RED and GREEN fluorescence levels were quantified.

Expected Results: The following results were obtained by flow cytometry analysis using GREEN (phenotype stain, 488 nm excitation and 525/40 nm emission filter) and DEEP RED (tag, 638 nm excitation and 660/10 nm emission filter) gates.

Test condition B showed enrichment for Low Stain Low Tag and High Stain High Tag populations (Chi-squared of 317 and p-value<0.0001 for Condition B) where expected while no such enrichment was observed in control condition A.

TABLE 18

| | Condition A | Condition B |
|---|---|---|
| High Stain High Tag | 7 (<1%) | 502 (16%) |
| High Stain Low Tag | 299 (38%) | 810 (26%) |
| Low Stain High Tag | 6 (<1%) | 202 (6.5%) |
| Low Stain Low Tag | 465 (60%) | 1599 (51%) |
| Total Counts | 777 | 3113 |

### Example 15

### Phenotype Identification and Cell Tagging Using Bleaching as a Tagging Method

### Experimental Description

Experimental Conditions: The wells of the MTP are divided into two conditions, each with replicates:

TABLE 19

| | Condition A | Condition B |
|---|---|---|
| Phenotypes | High Stain and | High Stain and |
| | Low Stain | Low Stain |
| Label | With Label | With Label |
| Tagging: Cells intended to be | None | High Stain |
| irradiated with high dose | | |

For the purpose of this Example, if not explicitly noted, all areas may be performed as described in Example 7. The following areas deviate from the procedure in Example 7. This workflow is depicted as a scheme in FIG. 12—with the exception of only two bins being created in this example.

Phenotypic Cell Staining: Cultured MDA-MB-231 cells were divided into two samples. One sample was treated with a working solution of CellTracker™ Red (prepared by dissolving lyophilized CellTracker™ product in DMSO to a stock concentration of 10 mM and then diluting the resultant solution in serum free medium to 5 uM or 0.5-25 uM, followed by prewarming to 37° C.). The second sample was treated with the same working solution without the CellTracker™ product. Both aliquots were incubated 30 min or 15-45 minutes under growth conditions. Cells were then washed to remove the CellTracker™ and dissociated (5 min, accutase at 37 deg C.). The two samples were then mixed, diluted with media and seeded into the wells of a 96 well MTP at a seeding density of 1,000-30,000 cells per well. The cells were then incubated in growth conditions for 1-3 days to form a monolayer or similar structure compatible with imaging.

Cell Labeling: For this example, the mixed cells were labelled with a green fluorescent amphiphile. The cells were labelled with a fluorescein conjugated to an amphiphile, DSPE-PEG₁₀₀-Amine. The dye labelled amphiphile was synthesized by reacting NHS-fluorescein (1.2-2 molar equivalents) with DSPE-PEG₁₀₀-Amine (1 molar equivalent) in triethylamine (2 molar equivalents) in DMSO. The dye conjugated amphiphile was used without further purification. The fluorescent amphiphile was kept as a stock in DMSO 2.5 mM or a range of 0.1 mM-10 mM and then diluted using buffer (such as PBS), media, or serum free media to a working concentration or 83 uM, 5 uM or between 0.1 nM and 100 uM. The cells were then treated with the working concentration of the label for 30 min at room temperature or 37 deg C. The cells were then washed 1-3 times with media.

### Phenotypic Cell Selection

Phenotypic Analysis: To characterize phenotype, as high or low intensity RED stain, cell segmentation is performed by common means. For example, by applying a pixel value threshold (obtained from Otsu or similar methods) to obtain a binary mask followed by applying morphological filters to better align the boundary of the masks with the image of individual cells. Individual cell masks are then identified and labeled in software to represent individual cells. Other common methods for cell segmentation are available in commercial or open-source image analysis software packages (Cell Profiler, GE Harmony, Molecular Devices IN Carta and others). The Cell ID and the reference of pixels associated with that cell (either in pixel map, coordinates, or as reference to label mask values) are then collated for all the cells in a lookup table. The total pixel intensity of each identified cell is then quantified by summing the pixel value of the resultant image from the pixel-wise multiplication of the fluorescence image with the mask of the identified cell. This total pixel value is divided by the value sum of the mask of the identified cell (i.e. cell area) to produce the average pixel intensity of the identified cell in that fluorescence channel. Phenotype of each cell is reported as the average or total pixel value of the RED fluorescence channel and added to the lookup table for each identified cell.

Specific Cell Selection: A binary phenotype for each cell is then defined by applying a user-selected threshold, Raw Phenotypic Threshold, on the total or average pixel intensity values of the stain channel in the lookup table, thereby creating a selection of cells with a phenotype of high-RED (with total or average pixel intensity above the threshold) and low-RED (with total or average pixel intensity below the threshold). An intended irradiation dose is determined for each of the subpopulation to enable sufficient distinction of tags in irradiated populations.

### Cell Tagging

Irradiation Event was performed as described in Example 7 except a different pre-set maximum irradiation time 4 minutes (or 8 minutes or 0.1 to 600 seconds in other embodiments) and an irradiation channel of 475/28 nm (or a wavelength within UV-VIS-NIR range of 190-3200 nm in embodiments) was used.

### Fluorescence Calibration Scan

Identification and Calibration of Tag Thresholds: Thresholds in the tag channel that best separate the two separate the phenotypic populations low-RED and high-RED are identified as Raw Tag Threshold and inputted as independent variables into the function described by Source Calibration Coefficients to obtain Calibrated Tag Threshold values.

### Downstream Processing

Analysis or Sorting: The flow cytometry buffer was PBS or a buffer that includes one or more of 0.5% BSA, 5 mM EDTA, or 12.5 mM HEPES. Cells resuspended in the flow cytometry buffer are run through FACS and sorting is done by using the Calibrated Tag Flow Gate values on the fluorescence channel corresponding to the tag fluorescence (Green, using a 488 nm laser), forming high-GREEN and low-Green populations. During sorting the fluorescence level of each cell in the channel corresponding to the cell stain used for phenotypic assessment is also measured (RED using a 594 nm laser).

Data Analysis: Using single cell data from the lookup table created during the Phenotypic Cell Selection process, the number of cells with RED and GREEN fluorescence levels above and below the raw fluorescence thresholds prior to any tagging are quantified.

Results: The following results were obtained by flow cytometry. Test condition B shows enrichment for the Low Stain High Tag and High Stain Low Tag populations (Chi-squared of 4.287 and p-value<0.0384 for Condition B) where expected while enrichment of High Stain Low Tag was not observed in control condition A.

TABLE 20

| | Condition A | Condition B |
|---|---|---|
| High Stain High Tag | 737(38%) | 390 (16%) |
| High Stain Low Tag | 17 (<1%) | 253 (11%) |
| Low Stain High Tag | 1134 (59%) | 1130 (48%) |
| Low Stain Low Tag | 52 (2.7%) | 602 (25%) |
| Total Count | 1940 | 2375 |

### Example 16

### Phenotype Identification and Cell Tagging Using Photoactivated Expressed Proteins

### Experimental Description

Experimental Conditions: The wells of the MTP are divided into five conditions, each in triplicate:

TABLE 21

| | Condition A | Condition B | Condition C | Condition D | Condition E |
|---|---|---|---|---|---|
| Phenotypes | High Stain and | High Stain and | High Stain and | High Stain and | High Stain and |
| | Low Stain | Low Stain | Low Stain | Low Stain | Low Stain |
| Label | Control, No | Control, No | With | With | With |
| | PAmCHERRY | PAmCHERRY | PAmCHERRY | PAmCHERRY | PAmCHERRY |
| Tagging: Cells intended | High Stain | Low Stain | None | High Stain | Low Stain |
| to be irradiated | | | | | |

For the purpose of this Example, the steps of cell dissociation may be performed as described in Example 7. The following areas deviate from Example 7.

Phenotypic Cell Staining: Cultured cells (e.g., MDA-MB-231, HeLa, MCF7, HEK293, or Mouse NSCs) expressing PAmCHERRY are divided into two samples. One sample is treated with a working solution of CellTracker™ Green (prepared by dissolving lyophilized CellTracker™ product in DMSO to a stock concentration of 10 mM and then diluting the resultant solution in serum free medium to 0.5-25 uM, followed by prewarming to 37° C.). The second sample is treated with the same working solution without the CellTracker™ product. Both aliquots are incubated 15-45 minutes under growth conditions. Cells are then washed to remove the CellTracker™. The two samples are then dissociated and mixed and seeded into the wells of a 96 well MTP at a seeding density of 1,000-30,000 cells per well. The cells are then incubated in growth conditions for 1-3 days to form a monolayer or similar structure compatible with imaging.

### Cell Selection

Nuclear Staining: To assist with cell segmentation, the cells are stained with a nuclear dye compatible with live cells and cell division as required by downstream processes. The dye will be selected to be compatible with the spectral constraints of other fluorescent stains, labels and tags in the experiment (e.g. DRAQ5). If DRAQ5 is used, it is added in cell media at final concentrations ranging from 0.1 to 20 M. Cells are then incubated for 5-30 minutes at room temperature.

### Phenotypic Cell Selection

Phenotypic Analysis: To characterize phenotype, as high or low intensity green stain, cell segmentation is performed by common means. For example, by applying a pixel value threshold (obtained from Otsu or similar methods) to obtain a binary mask followed by applying morphological filters to better align the boundary of the masks with the image of individual cells. Individual cell masks are then identified and labeled in software to represent individual cells. Other common methods for cell segmentation are available in commercial or open-source image analysis software packages (Cell Profiler, GE Harmony, Molecular Devices IN Carta and others). The Cell ID and the reference of pixels associated with that cell (either in pixel map, coordinates, or as reference to label mask values) are then collated for all the cells in a lookup table. The total pixel intensity of each identified cell is then quantified by summing the pixel value of the resultant image from the pixel-wise multiplication of the fluorescence image with the mask of the identified cell. This total pixel value is divided by the value sum of the mask of the identified cell (i.e. cell area) to produce the average pixel intensity of the identified cell in that fluorescence channel. Phenotype of each cell is reported as the average or total pixel value of the GREEN fluorescence channel and added to the lookup table for each identified cell.

Specific Cell Selection: The “high green phenotype” cells in Conditions A (control) and D are assigned a high irradiation dose to turn “on” the PAmCherry. In Conditions B (control) and E, the “low green phenotype” cells are assigned a high irradiation dose to turn “on” in those samples. Condition C remains unirradiated.

### Cell Tagging

### Fluorescence Calibration Scan

Identification and Calibration of Tag Thresholds: Thresholds in the tag channel that best separate the two separate the phenotypic populations low-GREEN and high-GREEN are identified as Raw Tag Threshold and inputted as independent variables into the function described by Source Calibration Coefficients to obtain Calibrated Tag Threshold values.

### Downstream Processing

Sorting: The flow cytometry buffer is PBS or a buffer that includes one or more of 0.5% BSA, 5 mM EDTA, 12.5 mM HEPES, or a similar sorting compatible buffer. Cells resuspended in the flow cytometry buffer are run through FACS and sorting is done by using the Calibrated Tag Flow Gate values on the fluorescence channel corresponding to the tag fluorescence (Red, using a 564 nm or similar laser), forming high-RED and low-RED populations. During sorting the fluorescence level of each cell in the channel corresponding to the cell stain used for phenotypic assessment is also measured (GREEN using a 488 nm or similar laser).

Data Analysis: Using single cell data from the lookup table created during the Phenotypic Cell Selection process, the number of cells with RED and GREEN fluorescence levels above and below the raw fluorescence thresholds prior to any tagging are quantified and labeled per table below:

TABLE 22

| | GREEN Fluorescence ≤ | GREEN Fluorescence > |
|---|---|---|
| | Raw Phenotypic | Raw Phenotypic |
| | Threshold | Threshold |
| RED ≤ Raw Tag | grₚᵣₑ | Grₚᵣₑ |
| Threshold | | |
| RED > Raw Tag | gRₚᵣₑ | GRₚᵣₑ |
| Threshold | | |

Expected Results: The following results are expected for each of the experimental conditions.

TABLE 23

| | Condition A | Condition B | Condition C | Condition D | Condition E |
|---|---|---|---|---|---|
| Expected | (grₚₒₛₜ/Grₚₒₛₜ) ~= | (grₚₒₛₜ/Grₚₒₛₜ) ~= | (GRₚₒₛₜ/Grₚₒₛₜ) ~= | (GRₚₒₛₜ/Grₚₒₛₜ) > | (gRₚₒₛₜ/grₚₒₛₜ) > |
| Result | (grₚᵣₑ/Grₚᵣₑ) | (grₚᵣₑ/Grₚᵣₑ) | (GRₚᵣₑ/Grₚᵣₑ) | (GRₚᵣₑ/Grₚᵣₑ) | (gRₚᵣₑ/grₚᵣₑ) |
| | and | and | and | and | and |
| | gRₚᵣₑ, GRₚᵣₑ, | gRₚᵣₑ, GRₚᵣₑ, | (gRₚₒₛₜ/grₚₒₛₜ) ~= | (gRₚₒₛₜ/grₚₒₛₜ) ~= | (GRₚₒₛₜ/Grₚₒₛₜ) ~= |
| | gRₚₒₛₜ, and | gRₚₒₛₜ, and | (gRₚᵣₑ/grₚᵣₑ) | (gRₚᵣₑ/grₚᵣₑ) | (GRₚᵣₑ/Grₚᵣₑ) |
| | GRₚₒₛₜ ~= 0 | GRₚₒₛₜ ~= 0 | | | |

### Example 17

### Phenotype Identification and Cell Tagging Using Uncaging of Photoblocked Oligonucleotides Followed by Enzymatic Cleavage

### Experimental Description

Experimental Conditions: The wells of the MTP were divided into three conditions with replicates:

TABLE 24

| | Condition A | Condition B | Condition C |
|---|---|---|---|
| Phenotypes | High Stain | High Stain | High Stain |
| | and Low | and Low | and Low |
| | Stain | Stain | Stain |
| Label | With Label | With Label | With Label |
| Tagging: Cells intended | None | All Cells | High Stain |
| to be irradiated with | | | |
| high dose | | | |

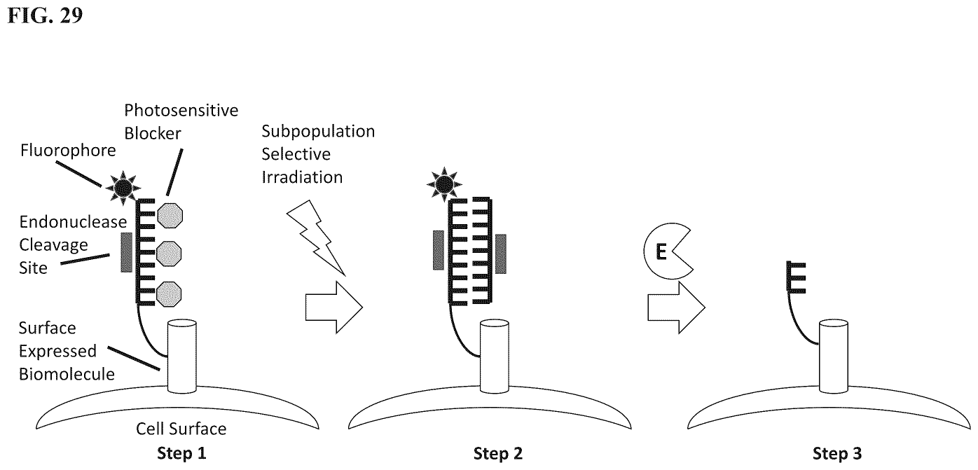

For the purpose of this Example, if not explicitly noted, all areas may be performed as described in Example 7. The following areas deviate from the procedure in Example 7. The scheme in FIG. 29 illustrates the following example.

Phenotypic Cell Staining: Cultured MDA-MB-231 cells were split into two samples. One sample was treated with a working solution of CellTracker™ Green (prepared by dissolving lyophilized CellTracker™ product in DMSO to a stock concentration of 10 mM or 0.1-10 mM and then diluting the resultant solution in serum free medium to 10 uM or 0.5-25 uM, followed by prewarming to 37° C.). The second sample was treated with the same working solution without the CellTracker™ product. Both aliquots were incubated for 30 min or 15-45 minutes under growth conditions. The two samples were then dissociated, mixed and seeded into the wells of a 96 well MTP at a seeding density of 1,000-30,000 cells per well. The cells were then incubated in growth conditions for 1-3 days to form a monolayer or similar structure compatible with imaging.

Cell Labeling: The stained cells were incubated with a photoblocked, oligonucleotide tagged antiCD44 antibody (1:20 dilution or a 1:10-1:200 dilution of a 2 mg/mL or a 0.1 mg/mL-2 mg/mL stock solution in buffer). The oligonucleotide tagged antibody was synthesized according to the Thunderlink oligonucleotide conjugation kit using an amine terminated oligonucleotide. The oligonucleotide was made up of an AseI endonuclease cleavage site, a Cy5 fluorophore, and photoblocking groups. After incubating the cells with the photoblocked oligonucleotide-antibody conjugate for 30 min or 5-90 min at room temperature or 37 deg the cells were then washed 1-3 times with buffer or media.

### Cell Selection

Nuclear Staining: No nuclear staining was performed.

### Phenotypic Cell Selection and Cell Tagging

Specific Cell Selection: The cells are washed and in Conditions C, the “high GREEN phenotype” cells were assigned to have a high intended irradiation dose whereas the “low GREEN phenotype” cells were assigned to have a low intended irradiation dose or no dose at all. Condition A remained unirradiated to serve as a negative control. Condition B received a dose of light across the entire well to serve as a positive control.

Enzymatic Cleavage: All cells were then treated with a solution of oligonucleotide complementary to the oligonucleotide-antibody conjugate 2 uM or a range of 0.1-10 uM, AseI restriction endonuclease (2× recommended amount or 1×-5× volume), and NEB provided 3.1 buffer (1× recommended amount). The complementary oligonucleotide shares the complement AseI cleavage sequence to that of the antibody-oligonucleotide conjugate. The enzyme, complementary oligo and antibody stained cells were incubated at 37 deg C. or room temperature for 30 min or 15-45 min. The cells were washed 1-3 times with buffer, media, or serum free media.

### Downstream Processing

Analysis by Flow Cytometry: The flow cytometry buffer was PBS or a buffer that includes one or more of 0.5% BSA, 5 mM EDTA, 12.5 mM HEPES, or other flow compatible buffer. Cells resuspended in the flow cytometry buffer were run through flow cytometry values on the fluorescence channel corresponding to the tag fluorescence (DEEP RED, using a 633 nm or similar laser), forming high-DEEP RED and low-DEEP RED populations. During analysis the fluorescence level of each cell in the channel corresponding to the cell stain used for phenotypic assessment is also measured (GREEN using a 488 nm or similar laser).

Data Analysis: Using single cell data from the lookup table created during the Phenotypic Cell Selection process, the number of cells with DEEP RED and GREEN fluorescence levels were quantified.

Results: The following results were obtained by flow cytometry analysis using GREEN (phenotype stain, 488 nm excitation and 525/40 nm emission filter) and DEEP RED (tag, 638 nm excitation and 660/10 nm emission filter) gates.

Test condition C showed enrichment for Low Stain High Tag and High Stain Low Tag populations (Chi-squared of 28 and p-value<0.0001 for Condition C) where expected while no such enrichment was observed in control condition A.

TABLE 25

| | Condition A | Condition B | Condition C |
|---|---|---|---|
| High Stain High Tag | 598 (24%) | 142 (15%) | 191 (10%) |
| High Stain Low Tag | 369 (14%) | 164 (17%) | 318 (18%) |
| Low Stain High Tag | 818 (32%) | 144 (16%) | 673 (37%) |
| Low Stain Low Tag | 730 (29%) | 481 (52%) | 638 (35%) |
| Total Counts | 2515 | 931 | 1820 |

### EMBODIMENTS

Embodiment 1: A method of irradiating a selected sub-population of cells within a population of cells comprising:
a) simultaneously irradiating each of a first selected sub-population of cells within a population of cells within a first digital image of a first microscope field of view with a first dose of light, thereby forming a first irradiated sub-population of cells and a remainder of cells within said population of cells, wherein at least a portion of said remainder of cells within said population of cells are labeled with the same photosensitive label as the first selected sub-population of cells, wherein a portion of the first selected sub-population of cells comprises a first cellular phenotype not present in a portion of the remainder of cells within said population of cells; and
b) quantitating said first irradiated sub-population of cells or separating said first irradiated sub-population of cells from said remainder of cells.

Embodiment 2. The method of embodiment 1, wherein said population of cells comprises a second selected sub-population of cells and wherein each of said second selected sub-population of cells within said population of cells is simultaneously irradiated with a second dose of light, thereby forming a second irradiated sub-population of cells.

Embodiment 3. The method of embodiment 2, wherein said first dose of light and said second dose of light are the same or different.

Embodiment 4. The method of embodiment 3, wherein said first selected sub-population of cells and said second selected sub-population of cells are simultaneously irradiated.

Embodiment 5. The method of one of embodiments 2 to 4, wherein said first dose of light corresponds to a first length of irradiation time for which said first selected sub-population of cells is irradiated, and wherein said second dose of light corresponds to a second length of irradiation time for which the said second selected sub-population of cells is irradiated.

Embodiment 6. The method of embodiment 5, wherein said first length of irradiation time and said second length of irradiation time are the same or different.

Embodiment 7. The method of one of embodiments 5 to 6, wherein said first length of irradiation time is shorter or longer than said second length of irradiation time.

Embodiment 8. The method of one of embodiments 5 to 7, wherein said first length of irradiation time and said second length of irradiation time start at a same timepoint or at different timepoints.

Embodiment 9. The method of one of embodiments 5 to 8, wherein said first length of irradiation time and said second length of irradiation time end at a same timepoint or at different timepoints.

Embodiment 10. The method of one of embodiments 5 to 9, wherein said first length of irradiation time starts before or after said second length of irradiation time.

Embodiment 11. The method of one of embodiments 2 to 10, wherein said first dose of light corresponds to a first intensity of light at which said first selected sub-population of cells is irradiated, and wherein said second dose of light corresponds to a second intensity of light at which the second selected sub-population of cells is irradiated.

Embodiment 12. The method of embodiment 11, wherein said first intensity of light and said second intensity of light are the same or different.

Embodiment 13. The method of one of embodiments 10 to 12, wherein said first dose of light and said second dose of light correspond to a duty cycle of an irradiation unit irradiating said first selected sub-population of cells and said second selected sub-population of cells.

Embodiment 14. The method of embodiment 13, wherein said irradiation unit includes a light source.

Embodiment 15. The method of embodiment 14, wherein said light source comprises one or more of a light emitting diode (LED), a laser, an arc lamp, or an incandescent lamp.

Embodiment 16. The method of one of embodiments 14 to 15, wherein said irradiation unit further includes a light patterning mechanism.

Embodiment 17. The method of embodiment 16, wherein said light patterning mechanism includes one or more of a digital micromirror device (DMD).

Embodiment 18. The method of one of embodiments 1 to 17, wherein at least 99% of said remainder of cells are not irradiated at said first dose of light at the time each of said first selected sub-population of cells is irradiated at said first dose of light.

Embodiment 19. The method of one of embodiments 2 to 18, wherein at least 99% of said remainder of cells are not irradiated at said second dose of light at the time each of said second selected sub-population of cells is irradiated at said second dose of light.

Embodiment 20. The method of one of embodiments 1 to 19, wherein said population of cells comprises an additional selected sub-population of cells and wherein each cell of said additional selected sub-population of cells within said population of cells is simultaneously irradiated at an additional dose of light, thereby forming an additional irradiated sub-population of cells.

Embodiment 21. The method of embodiment 20, wherein said first dose of light and said additional dose of light are the same or different.

Embodiment 22. The method of embodiment 21, wherein said first selected sub-population of cells and said additional selected sub-population of cells are simultaneously irradiated.

Embodiment 23. The method of one of embodiments 20 to 22, wherein said first selected sub-population of cells is simultaneously irradiated at a starting timepoint t1.

Embodiment 24. The method of one of embodiments 20 to 23, wherein said additional selected sub-population of cells is simultaneously irradiated at an additional starting timepoint t.

Embodiment 25. The method of embodiment 24, wherein said t1 and said t are the same.

Embodiment 26. The method of embodiment 24, wherein said t1 precedes said t.

Embodiment 27. The method of one of embodiments 20 to 26, wherein said first selected sub-population of cells is simultaneously irradiated for a first length of irradiation time.

Embodiment 28. The method of embodiment 27, wherein said first length of irradiation time ends at an endpoint tf1.

Embodiment 29. The method of one of embodiments 20 to 28, wherein said additional selected sub-population of cells is simultaneously irradiated for an additional length of irradiation time.

Embodiment 30. The method of embodiment 29, wherein said additional length of irradiation time ends at an endpoint tfa.

Embodiment 31. The method of one of embodiments 28 to 30, wherein said endpoint tf1 and said endpoint tfa are the same or different.

Embodiment 32. The method of one of embodiments 29 to 31, wherein said first length of irradiation time and said additional length of irradiation time are the same.

Embodiment 33. The method of one of embodiments 29 to 31, wherein said first length of irradiation time and said additional length of irradiation time are different.

Embodiment 34. The method of one of embodiments 29 to 31, wherein said first length of irradiation time is shorter or longer relative to said additional length of irradiation time.

Embodiment 35. The method of one of embodiments 1 to 19, wherein said simultaneous irradiating is based on the location of said first selected sub-population of cells within said first digital image of a first microscope field of view.

Embodiment 36. The method of one of embodiments 1 to 19 or 35, wherein said simultaneous irradiating is further based on the location of said first selected sub-population of cells within a plurality of first digital images of a first microscope field of view.

Embodiment 37. The method of one of embodiments 2 to 19 or 35-36, wherein said simultaneous irradiating is based on the location of said second selected sub-population of cells within said first digital image of a first microscope field of view.

Embodiment 38. The method of one of embodiments 1 to 19 or 37, wherein said simultaneous irradiating is further based on the location of said second selected sub-population of cells within a plurality of digital images of a first microscope field of view.

Embodiment 39. The method of one of embodiments 1 to 38, wherein said first digital image is formed from a first raw projection image.

Embodiment 40. The method of embodiment 39, further comprising: selecting, based at least on a lookup table (LUT), said first selected sub-population of cells, the lookup table mapping each cell within said population of cells to one or more corresponding pixels in a first raw digital image, the pixels corresponding to the first selected sub-population of cells comprising a subset lookup table (LUT), and the first raw projection image being formed by assigning a desired irradiation value to each pixel included in the subset lookup table.

Embodiment 41. The method of any one of embodiments 39 to 40, wherein said first raw projection image is formed from the first raw digital image.

Embodiment 42. The method of embodiment 39 or 41, wherein said first raw projection image further comprises at least a portion of an additional microscope field of view.

Embodiment 43. The method of one of embodiments 2 to 42, wherein said second digital image is formed from a second raw projection image.

Embodiment 44. The method of embodiment 43, wherein said second raw projection image is formed from a second raw digital image.

Embodiment 45. The method of embodiment 43 or 44, wherein said second raw projection image further comprises at least a portion of an additional microscope field of view.

Embodiment 46. The method of one of embodiments 2 to 19 or 35 to 45, wherein said first digital image comprises at least a portion of a second microscope field of view.

Embodiment 47. The method of one of embodiments 2 to 19 or 35 to 46, wherein said simultaneous irradiating is further based on the location of said first selected sub-population of cells within a second digital image of a second microscope field of view.

Embodiment 48. The method of one of embodiments 2 to 19 or 35 to 47, wherein said simultaneous irradiating is further based on the location of said second selected sub-population of cells within a second digital image of a second microscope field of view.

Embodiment 49. The method of one of embodiments 1 to 19 or 35 to 48, wherein said population of cells is comprised in a sample and wherein said sample moves with a movement velocity.

Embodiment 50. The method of embodiment 49, further comprising: determining, based at least on said movement velocity of said sample, one or more transformations for forming said first digital image or said second digital image; and applying said one or more transformations to form said first digital image or said second digital image.

Embodiment 51. The method of embodiment 50, wherein said one or more transformations include cropping.

Embodiment 52. The method of any one of embodiments 50 to 51, wherein said one or more transformations include a geometric transformation.

Embodiment 53. The method of embodiment 52, wherein the geometric transformation includes one or more of a Euclidean transformation, an affine transformation, or a projective transformation.

Embodiment 54. The method of any one of embodiments 50 to 53, wherein the movement velocity is predetermined.

Embodiment 55. The method of any one of embodiments 50 to 54, further comprising:
determining said movement velocity of said sample at a first time and a second time;
determining, based at least on a first movement velocity of said sample at the first time, a first transformation for forming said first digital image; and
determining, based at least on a second movement velocity of said sample at the second time, a second transformation for forming said second digital.

Embodiment 56. The method of one of embodiments 49 to 51, wherein said sample is in an irradiation device.

Embodiment 57. The method of one of embodiments 2 to 56, wherein a portion of the second selected sub-population of cells comprises a second cellular phenotype not present in a portion of the remainder of cells within said population of cells.

Embodiment 58. The method of one of embodiments 2 to 57, further comprising quantitating said second irradiated sub-population of cells or separating said second irradiated sub-population of cells from said remainder of cells.

Embodiment 59. The method of embodiment 57, wherein said first cellular phenotype and said second cellular phenotype are different or the same.

Embodiment 60. The method of one of embodiments 1 to 59, wherein said portion of the first selected sub-population of cells comprising a first cellular phenotype is at least 50% of said first selected sub-population of cells.

Embodiment 61. The method of one of embodiments 1 to 60, wherein said portion of the first selected sub-population of cells comprising a first cellular phenotype is at least 90% of said first selected sub-population of cells.

Embodiment 62. The method of one of embodiments 1 to 61, wherein said portion of the first selected sub-population of cells comprising a first cellular phenotype is at least 99% of said first selected sub-population of cells.

Embodiment 63. The method of one of embodiments 1 to 59, wherein said portion of the remainder of cells within said population of cells wherein said first cellular phenotype is not present is at least 50% of said remainder of cells.

Embodiment 64. The method of one of embodiments 1 to 59 or 63, wherein said portion of the remainder of cells within said population of cells wherein said first cellular phenotype is not present is at least 90% of said remainder of cells.

Embodiment 65. The method of one of embodiments 1 to 59, 63 or 64, wherein said portion of the remainder of cells within said population of cells wherein said first cellular phenotype is not present in at least 99% of said remainder of cells.

Embodiment 66. The method of one of embodiments 2 to 65, wherein at least a portion of said remainder of cells within said population of cells are labeled with the same photosensitive label as said second selected sub-population of cells.

Embodiment 67. The method of one of embodiments 2 to 66, wherein said first selected sub-population of cells and said second selected sub-population of cells are labeled with the same photosensitive label.

Embodiment 68. The method of one of embodiments 2 to 66, wherein said first selected sub-population of cells is labeled with a first photosensitive label and said second selected sub-population of cells is labeled with a second photosensitive label.

Embodiment 69. The method of one of embodiments 1 to 68, wherein said portion of said remainder of cells within said population of cells labeled with the same photosensitive label is at least 50% of said remainder of cells.

Embodiment 70. The method of one of embodiments 1 to 69, said portion of said remainder of cells within said population of cells labeled with the same photosensitive label is at least 90% of said remainder of cells.

Embodiment 71. The method of one of embodiments 1 to 70, wherein said portion of said remainder of cells within said population of cells labeled with the same photosensitive label is at least 99% of said remainder of cells.

Embodiment 72. The method of one of embodiments 1 to 71, wherein said portion of said remainder of cells within said population of cells labeled with the same photosensitive label is 100% of said remainder of cells.

Embodiment 73. The method of one of embodiments 1 to 68, wherein a portion of said remainder of cells within said population of cells are not labeled with the same photosensitive label as the first selected sub-population of cells.

Embodiment 74. The method of one of embodiments 1 to 68, wherein a portion of said remainder of cells within said population of cells are unlabeled.

Embodiment 75. The method of one of embodiments 1 to 74, wherein said simultaneously irradiating activates said photosensitive label.

Embodiment 76. The method of one of embodiments 1 to 74 wherein said simultaneous irradiation deactivates said photosensitive label.

Embodiment 77. The method of one of embodiments 1 to 76, wherein said photosensitive label is attached to said first selected sub-population of cells or said remainder of cells through a chemical linker.

Embodiment 78. The method of embodiment 77, wherein said chemical linker is a covalent linker or a non-covalent linker.

Embodiment 79. The method of embodiment 77 or 78, wherein said chemical linker comprises a nucleic acid.

Embodiment 80. The method of one of embodiments 77-79, wherein said chemical linker comprises a double-stranded nucleic acid.

Embodiment 81. The method of one of embodiments 77-79, wherein said chemical linker comprises a unique molecular identifier (UMI).

Embodiment 82. The method of embodiment 78, wherein said non-covalent linker comprises an antibody.

Embodiment 83. The method of embodiment 82, wherein said non-covalent linker comprises an antibody-nucleic acid conjugate.

Embodiment 84. The method of embodiment 77, wherein said photosensitive label is a labeling oligonucleotide comprising a photosensitive blocking moiety.

Embodiment 85. The method of embodiment 84, wherein said labeling oligonucleotide comprises a unique molecular identifier (UMI).

Embodiment 86. The method of embodiment 84, wherein said simultaneous irradiation in a) further comprises deprotecting said labeling oligonucleotide thereby removing said photosensitive blocking moiety from said labeling oligonucleotide and forming a deprotected labeling oligonucleotide.

Embodiment 87. The method of embodiment 86, wherein said quantitating in b) further comprises
i) contacting said deprotected labeling oligonucleotide with a template oligonucleotide and a polymerase or a ligase thereby forming a barcoded oligonucleotide and
ii) detecting said barcoded oligonucleotide.

Embodiment 88. The method of embodiment 77, wherein said photosensitive label is a labeling oligonucleotide comprising a plurality of photosensitive blocking moieties each attached to a nucleotide of said labeling oligonucleotide.

Embodiment 89. The method of embodiment 88, wherein said simultaneous irradiation in a) further comprises deprotecting said labeling oligonucleotide thereby removing said plurality of photosensitive blocking moieties from said labeling oligonucleotide and forming a deprotected labeling oligonucleotide.

Embodiment 90. The method of embodiment 89, wherein said quantitating in b) further comprises
i) contacting said deprotected labeling oligonucleotide with a template oligonucleotide and a polymerase or a ligase thereby forming a barcoded oligonucleotide and
ii) detecting said barcoded oligonucleotide.

Embodiment 91. The method of any one of embodiments 1 to 90, wherein said photosensitive label comprises a fluorophore moiety.

Embodiment 92. The method of any one of embodiments 1 to 90, wherein said photosensitive label comprises one or more of a photolabile protecting groups.

Embodiment 93. The method of any one of embodiments 1 to 90, wherein said photosensitive label comprises a template oligonucleotide attached to a fluorophore moiety.

Embodiment 94. The method of any one of embodiments 1 to 90, wherein said photosensitive label comprises a template oligonucleotide hybridized to a labeling oligonucleotide, wherein the labeling oligonucleotide is attached to a fluorophore moiety.

Embodiment 95. The method of any one of embodiments 1 to 90, wherein said photosensitive label comprises a template oligonucleotide hybridized to a labeling oligonucleotide, wherein the template oligonucleotide is attached to a fluorophore moiety.

Embodiment 96. A method of selecting a sub-population of cells within a population of cells comprising:
a) simultaneously irradiating each of a first selected sub-population of cells within a population of cells within a first digital image of a first microscope field of view with a first dose of light, thereby forming a first non-irradiated sub-population of cells and a remainder of cells within said population of cells, wherein at least a portion of said remainder of cells within said population of cells are labeled with the same photosensitive label as the first selected sub-population of cells, wherein a portion of the first selected sub-population of cells comprises a first cellular phenotype not present in a portion of the remainder of cells within said population of cells; and
b) quantitating said non-irradiated sub-population of cells or separating said non-irradiated sub-population of cells from said remainder of cells.

Embodiment 97. The method of one of embodiments 1-96, wherein said population of cells is a population of prokaryotic cells.

Embodiment 98. The method of one of embodiments 1-96, wherein said population of cells is a population of eukaryotic cells.

Embodiment 99. The method of one of embodiments 1-98, wherein said population of cells comprises a population of adherent cells.

Embodiment 100. The method of one of embodiments 1-98, wherein said population of cells comprises a population of non-adherent cells.

Embodiment 101. The method of one of embodiments 1-98, wherein said population of cells comprises a population of adherent cells and a population of non-adherent cells.

Embodiment 102. The method of one of embodiments 1-98, wherein said population of cells is a population of adherent cells.

Embodiment 103. The method of one of embodiments 1-98, wherein said population of cells is a population of non-adherent cells.

What is claimed is:

1. A method of irradiating a selected sub-population of cells within a population of cells comprising:
a) simultaneously irradiating each of a first selected sub-population of cells within a population of cells within a first digital image of a first microscope field of view with a first dose of light, thereby forming a first irradiated sub-population of cells and a remainder of cells within said population of cells, wherein at least a portion of said remainder of cells within said population of cells are labeled with the same photosensitive label as the first selected sub-population of cells, wherein a portion of the first selected sub-population of cells comprises a first cellular phenotype not present in a portion of the remainder of cells within said population of cells; and
b) quantitating said first irradiated sub-population of cells or separating said first irradiated sub-population of cells from said remainder of cells.

2. The method of claim 1, wherein said population of cells comprises a second selected sub-population of cells and wherein each of said second selected sub-population of cells within said population of cells is simultaneously irradiated with a second dose of light, thereby forming a second irradiated sub-population of cells.

3. The method of claim 2, wherein said first dose of light and said second dose of light are the same or different.

4. The method of claim 3, wherein said first selected sub-population of cells and said second selected sub-population of cells are simultaneously irradiated.

5. The method of claim 4, wherein said first dose of light corresponds to a first length of irradiation time for which said first selected sub-population of cells is irradiated, and wherein said second dose of light corresponds to a second length of irradiation time for which the said second selected sub-population of cells is irradiated.

6. The method of claim 5, wherein said first length of irradiation time and said second length of irradiation time are the same or different.

7. The method of claim 5, wherein said first length of irradiation time is shorter or longer than said second length of irradiation time.

8. The method of claim 5, wherein said first length of irradiation time and said second length of irradiation time start at a same timepoint or at different timepoints.

9. The method of claim 5, wherein said first length of irradiation time and said second length of irradiation time end at a same timepoint or at different timepoints.

10. The method of claim 5, wherein said first length of irradiation time starts before or after said second length of irradiation time.

11. The method of claim 5, wherein said first dose of light corresponds to a first intensity of light at which said first selected sub-population of cells is irradiated, and wherein said second dose of light corresponds to a second intensity of light at which the second selected sub-population of cells is irradiated.

12. The method of claim 11, wherein said first intensity of light and said second intensity of light are the same or different.

13. The method of claim 10, wherein said first dose of light and said second dose of light correspond to a duty cycle of an irradiation unit irradiating said first selected sub-population of cells and said second selected sub-population of cells.

14. The method of claim 13, wherein said irradiation unit includes a light source.

15. The method of claim 14, wherein said light source comprises one or more of a light emitting diode (LED), a laser, an arc lamp, or an incandescent lamp.

16. The method of claim 14, wherein said irradiation unit further includes a light patterning mechanism.

17. The method of claim 16, wherein said light patterning mechanism includes one or more of a digital micromirror device (DMD).

18. The method of claim 1, wherein at least 99% of said remainder of cells are not irradiated at said first dose of light at the time each of said first selected sub-population of cells is irradiated at said first dose of light.

19. The method of claim 2, wherein at least 99% of said remainder of cells are not irradiated at said second dose of light at the time each of said second selected sub-population of cells is irradiated at said second dose of light.

20. The method of claim 1, wherein said population of cells comprises an additional selected sub-population of cells and wherein each cell of said additional selected sub-population of cells within said population of cells is simultaneously irradiated at an additional dose of light, thereby forming an additional irradiated sub-population of cells.

21. The method of claim 20, wherein said first dose of light and said additional dose of light are the same or different.

22. The method of claim 21, wherein said first selected sub-population of cells and said additional selected sub-population of cells are simultaneously irradiated.

23. The method of claim 20, wherein said first selected sub-population of cells is simultaneously irradiated at a starting timepoint t1.

24. The method of one of claim 20, wherein said additional selected sub-population of cells is simultaneously irradiated at an additional starting timepoint t.

25. The method of claim 24, wherein said t1 and said t are the same.

26. The method of claim 24, wherein said t1 precedes said t.

27. The method of claim 20, wherein said first selected sub-population of cells is simultaneously irradiated for a first length of irradiation time.

28. The method of claim 27, wherein said first length of irradiation time ends at an endpoint tf1.

29. The method of claim 20, wherein said additional selected sub-population of cells is simultaneously irradiated for an additional length of irradiation time.

30. The method of claim 29, wherein said additional length of irradiation time ends at an endpoint tfa.

31. The method of claim 28, wherein said endpoint tf1 and said endpoint tfa are the same or different.

32. The method of claim 29, wherein said first length of irradiation time and said additional length of irradiation time are the same.

33. The method of claim 29, wherein said first length of irradiation time and said additional length of irradiation time are different.

34. The method of claim 29, wherein said first length of irradiation time is shorter or longer relative to said additional length of irradiation time.

35. The method of claim 1, wherein said simultaneous irradiating is based on the location of said first selected sub-population of cells within said first digital image of a first microscope field of view.

36. The method of claim 35, wherein said simultaneous irradiating is further based on the location of said first selected sub-population of cells within a plurality of first digital images of a first microscope field of view.

37. The method of claim 35, wherein said simultaneous irradiating is based on the location of said second selected sub-population of cells within said first digital image of a first microscope field of view.

38. The method of claim 37, wherein said simultaneous irradiating is further based on the location of said second selected sub-population of cells within a plurality of digital images of a first microscope field of view.

40. The method of claim 38, wherein said first digital image is formed from a first raw projection image.

41. The method of claim 40, further comprising:
selecting, based at least on a lookup table (LUT), said first selected sub-population of cells, the lookup table mapping each cell within said population of cells to one or more corresponding pixels in a first raw digital image, the pixels corresponding to the first selected sub-population of cells comprising a subset lookup table (LUT), and the first raw projection image being formed by assigning a desired irradiation value to each pixel included in the subset lookup table.

42. The method of claim 40, wherein said first raw projection image is formed from the first raw digital image.

43. The method of claim 40, wherein said first raw projection image further comprises at least a portion of an additional microscope field of view.

44. The method of claim 43, wherein said second digital image is formed from a second raw projection image.

45. The method of claim 44, wherein said second raw projection image is formed from a second raw digital image.

46. The method of claim 44, wherein said second raw projection image further comprises at least a portion of an additional microscope field of view.

47. The method of claim 35, wherein said first digital image comprises at least a portion of a second microscope field of view.

48. The method of claim 47, wherein said simultaneous irradiating is further based on the location of said first selected sub-population of cells within a second digital image of a second microscope field of view.

49. The method of claim 48, wherein said simultaneous irradiating is further based on the location of said second selected sub-population of cells within a second digital image of a second microscope field of view.

50. The method of claim 35, wherein said population of cells is comprised in a sample and wherein said sample moves with a movement velocity.

51. The method of claim 50, further comprising:
determining, based at least on said movement velocity of said sample, one or more transformations for forming said first digital image or said second digital image; and
applying said one or more transformations to form said first digital image or said second digital image.

52. The method of claim 51, wherein said one or more transformations include cropping.

53. The method of claim 51, wherein said one or more transformations include a geometric transformation.

54. The method of claim 53, wherein the geometric transformation includes one or more of a Euclidean transformation, an affine transformation, or a projective transformation.

55. The method of claim 51, wherein the movement velocity is predetermined.

56. The method of claim 51, further comprising:
determining said movement velocity of said sample at a first time and a second time;
determining, based at least on a first movement velocity of said sample at the first time, a first transformation for forming said first digital image; and
determining, based at least on a second movement velocity of said sample at the second time, a second transformation for forming said second digital.

57. The method of claim 50, wherein said sample is in an irradiation device.

58. The method of claim 2, wherein a portion of the second selected sub-population of cells comprises a second cellular phenotype not present in a portion of the remainder of cells within said population of cells.

59. The method of claim 2, further comprising quantitating said second irradiated sub-population of cells or separating said second irradiated sub-population of cells from said remainder of cells.

60. The method of claim 58, wherein said first cellular phenotype and said second cellular phenotype are different or the same.

61. The method of claim 60, wherein said portion of the first selected sub-population of cells comprising a first cellular phenotype is at least 50% of said first selected sub-population of cells.

62. The method of claim 61, wherein said portion of the first selected sub-population of cells comprising a first cellular phenotype is at least 90% of said first selected sub-population of cells.

63. The method of claim 62 wherein said portion of the first selected sub-population of cells comprising a first cellular phenotype is at least 99% of said first selected sub-population of cells.

64. The method of claim 60, wherein said portion of the remainder of cells within said population of cells wherein said first cellular phenotype is not present is at least 50% of said remainder of cells.

65. The method of claim 64, wherein said portion of the remainder of cells within said population of cells wherein said first cellular phenotype is not present is at least 90% of said remainder of cells.

66. The method of claim 65, wherein said portion of the remainder of cells within said population of cells wherein said first cellular phenotype is not present in at least 99% of said remainder of cells.

67. The method of claim 66, wherein at least a portion of said remainder of cells within said population of cells are labeled with the same photosensitive label as said second selected sub-population of cells.

68. The method of claim 67, wherein said first selected sub-population of cells and said second selected sub-population of cells are labeled with the same photosensitive label.

69. The method of claim 67, wherein said first selected sub-population of cells is labeled with a first photosensitive label and said second selected sub-population of cells is labeled with a second photosensitive label.

70. The method of claim 69, wherein said portion of said remainder of cells within said population of cells labeled with the same photosensitive label is at least 50% of said remainder of cells.

71. The method of claim 70, said portion of said remainder of cells within said population of cells labeled with the same photosensitive label is at least 90% of said remainder of cells.

72. The method of claim 71, wherein said portion of said remainder of cells within said population of cells labeled with the same photosensitive label is at least 99% of said remainder of cells.

73. The method of claim 72, wherein said portion of said remainder of cells within said population of cells labeled with the same photosensitive label is 100% of said remainder of cells.

74. The method of claim 68, wherein a portion of said remainder of cells within said population of cells are not labeled with the same photosensitive label as the first selected sub-population of cells.

75. The method of claim 69, wherein a portion of said remainder of cells within said population of cells are unlabeled.

76. The method of claim 75, wherein said simultaneously irradiating activates said photosensitive label.

77. The method of claim 75 wherein said simultaneous irradiation deactivates said photosensitive label.

78. The method of claim 77, wherein said photosensitive label is attached to said first selected sub-population of cells or said remainder of cells through a chemical linker.

79. The method of claim 78, wherein said chemical linker is a covalent linker or a non-covalent linker.

80. The method of claim 79, wherein said chemical linker comprises a nucleic acid.

81. The method of claim 80, wherein said chemical linker comprises a double-stranded nucleic acid.

82. The method of claim 80, wherein said chemical linker comprises a unique molecular identifier (UMI).

83. The method of claim 79, wherein said non-covalent linker comprises an antibody.

84. The method of claim 83, wherein said non-covalent linker comprises an antibody-nucleic acid conjugate.

85. The method of claim 78, wherein said photosensitive label is a labeling oligonucleotide comprising a photosensitive blocking moiety.

86. The method of claim 85, wherein said labeling oligonucleotide comprises a unique molecular identifier (UMI).

87. The method of claim 85, wherein said simultaneous irradiation in a) further comprises deprotecting said labeling oligonucleotide thereby removing said photosensitive blocking moiety from said labeling oligonucleotide and forming a deprotected labeling oligonucleotide.

88. The method of claim 87, wherein said quantitating in b) further comprises
i) contacting said deprotected labeling oligonucleotide with a template oligonucleotide and a polymerase or a ligase thereby forming a barcoded oligonucleotide and
ii) detecting said barcoded oligonucleotide.

89. The method of claim 78, wherein said photosensitive label is a labeling oligonucleotide comprising a plurality of photosensitive blocking moieties each attached to a nucleotide of said labeling oligonucleotide.

90. The method of claim 89, wherein said simultaneous irradiation in a) further comprises deprotecting said labeling oligonucleotide thereby removing said plurality of photosensitive blocking moieties from said labeling oligonucleotide and forming a deprotected labeling oligonucleotide.

91. The method of claim 90, wherein said quantitating in b) further comprises
i) contacting said deprotected labeling oligonucleotide with a template oligonucleotide and a polymerase or a ligase thereby forming a barcoded oligonucleotide and
ii) detecting said barcoded oligonucleotide.

92. The method of claim 1, wherein said photosensitive label comprises a fluorophore moiety.

93. The method of claim 1, wherein said photosensitive label comprises one or more of a photolabile protecting groups.

94. The method of claim 1, wherein said photosensitive label comprises a template oligonucleotide attached to a fluorophore moiety.

95. The method of claim 1, wherein said photosensitive label comprises a template oligonucleotide hybridized to a labeling oligonucleotide, wherein the labeling oligonucleotide is attached to a fluorophore moiety.

96. The method of claim 1, wherein said photosensitive label comprises a template oligonucleotide hybridized to a labeling oligonucleotide, wherein the template oligonucleotide is attached to a fluorophore moiety.

97. A method of selecting a sub-population of cells within a population of cells comprising:
a) simultaneously irradiating each of a first selected sub-population of cells within a population of cells within a first digital image of a first microscope field of view with a first dose of light, thereby forming a first non-irradiated sub-population of cells and a remainder of cells within said population of cells, wherein at least a portion of said remainder of cells within said population of cells are labeled with the same photosensitive label as the first selected sub-population of cells, wherein a portion of the first selected sub-population of cells comprises a first cellular phenotype not present in a portion of the remainder of cells within said population of cells; and
b) quantitating said non-irradiated sub-population of cells or separating said non-irradiated sub-population of cells from said remainder of cells.

98. The method of claim 97, wherein said population of cells is a population of prokaryotic cells.

99. The method of claim 97, wherein said population of cells is a population of eukaryotic cells.

100. The method of claim 99, wherein said population of cells comprises a population of adherent cells.

101. The method of claim 99, wherein said population of cells comprises a population of non-adherent cells.

102. The method of claim 99, wherein said population of cells comprises a population of adherent cells and a population of non-adherent cells.

103. The method of claim 99, wherein said population of cells is a population of adherent cells.

104. The method of claim 99, wherein said population of cells is a population of non-adherent cells.
